SEQUENCE LISTING (131 sequences)

SEQ ID NO: 1 - DNA, 20 bp, Artificial Sequence
  Synthetic_GAPDH-Forward
ctcctccacctttgacgctg

SEQ ID NO: 2 - DNA, 20 bp, Artificial Sequence
  Synthetic_GAPDH-Reverse
tcctcttgtgctcttgctgg

SEQ ID NO: 3 - DNA, 20 bp, Artificial Sequence
  Synthetic_PKR-Forward
gaggggaatgatgtgattgg

SEQ ID NO: 4 - DNA, 20 bp, Artificial Sequence
  Synthetic_PKR-Reverse
ctgggctgtcacttctagcc

SEQ ID NO: 5 - DNA, 21 bp, Artificial Sequence
  Synthetic_Mcl-1-Forward
ctctcatttcttttggtgcct

SEQ ID NO: 6 - DNA, 20 bp, Artificial Sequence
  Synthetic_Mcl-1-Reverse
attcctgatgccaccttcta

SEQ ID NO: 7 - DNA, 20 bp, Artificial Sequence
  Synthetic_Bcl-xL-Forward
tccccatggcagcagtaaag

SEQ ID NO: 8 - DNA, 25 bp, Artificial Sequence
  Synthetic_Bcl-xL-Reverse
tccacaaaagtatcctgttcaaagc

SEQ ID NO: 9 - DNA, 19 bp, Artificial Sequence
  Synthetic_Bcl-2-Forward
gagagtgctgaagattgat

SEQ ID NO: 10 - DNA, 19 bp, Artificial Sequence
  Synthetic_Bcl-2-Reverse
atcaatcttcagcactctc

SEQ ID NO: 11 - DNA, 46 bp, Artificial Sequence
  Synthetic_12nt stem_HP
tgcatcgtccatccatctttaccagacagtgttaatggacgatgca

SEQ ID NO: 12 - DNA, 50 bp, Artificial Sequence
  Synthetic_14nt stem_HP
gttgcatcgtccatccatctttaccagacagtgttaatggacgatgcaac

SEQ ID NO: 13 - DNA, 54 bp, Artificial Sequence
  Synthetic_16nt stem_HP
tggttgcatcgtccatccatctttaccagacagtgttaatggacgatgcaacca

SEQ ID NO: 14 - DNA, 58 bp, Artificial Sequence
  Synthetic_18nt stem_HP
catggttgcatcgtccatccatctttaccagacagtgttaatggacgatgcaaccatg

SEQ ID NO: 15 - DNA, 62 bp, Artificial Sequence
  Synthetic_20nt stem_HP
accatggttgcatcgtccatccatctttaccagacagtgttaatggacgatgcaaccatggt

SEQ ID NO: 16 - DNA, 66 bp, Artificial Sequence
  Synthetic_22nt stem_HP
tgaccatggttgcatcgtccatccatctttaccagacagtgttaatggacgatgcaaccatggtca

SEQ ID NO: 17 - RNA, 22 nt, Artificial Sequence
  Synthetic_miR-141
uaacacugucugguaaagaugg

SEQ ID NO: 18 - DNA, 46 bp, Artificial Sequence
  Synthetic_HP-Mcl1-141
caagaggattatccatctttacctcacagtgttaataatcctcttg

SEQ ID NO: 19 - RNA, 19 nt, Artificial Sequence
  Synthetic_Mcl1_mRNA
gcaaguggcaagaggauua

SEQ ID NO: 20 - DNA, 45 bp, Artificial Sequence
  Synthetic_HP_Mcl1_M2_S12_miR-21
caagaggattattcaacatcagattgataagctataatcctcttg

SEQ ID NO: 21 - DNA, 46 bp, Artificial Sequence
  Synthetic_HP_Mcl1_M2_S12_miR-200a
caagaggattatacatcgttaccgtacagtgttaataatcctcttg

SEQ ID NO: 22 - DNA, 46 bp, Artificial Sequence
  Synthetic_HP_Mcl1_M2_S12_miR-200b
caagaggattattcatcattaccgagcagtattaataatcctcttg

SEQ ID NO: 23 - DNA, 47 bp, Artificial Sequence
  Synthetic_HP_Mcl1_M2_S12_miR-200c
caagaggattattccatcattaccttgcagtattaataatcctcttg

SEQ ID NO: 24 - RNA, 22 nt, Artificial Sequence
  Synthetic_miR-21
uagcuuaucagacugauguuga

SEQ ID NO: 25 - RNA, 22 nt, Artificial Sequence
  Synthetic_miR-200a
uaacacugucugguaacgaugu

SEQ ID NO: 26 - RNA, 22 nt, Artificial Sequence
  Synthetic_miR-200b
uaauacugccugguaaugauga

SEQ ID NO: 27 - RNA, 23 nt, Artificial Sequence
  Synthetic_miR-200c
uaauacugccggguaaugaugga

SEQ ID NO: 28 - RNA, 60 nt, Artificial Sequence
  Synthetic_Mcl-1 mRNA
acccuagcaaccuagccagaaaagcaaguggcaagaggauuauggcuaacaagaauaaau

SEQ ID NO: 29 - DNA, 50 bp, Artificial Sequence
  Synthetic_DNA HP
tatttctcattcccccatctttaccagacagtgttagggaatgagaaata

SEQ ID NO: 30 - DNA, 50 bp, Artificial Sequence; identical to SEQ ID NO: 29
  Synthetic_LNA HP,wherein nucleotides at positions 1-4 and 47-50 are modified with LNA

SEQ ID NO: 31 - DNA, 50 bp, Artificial Sequence; identical to SEQ ID NO: 29
  Synthetic_PS4 H, wherein nucleotides at positions 1-4 and 46-49 are modified with phosphorothioate linkage

SEQ ID NO: 32 - DNA, 50 bp, Artificial Sequence; identical to SEQ ID NO: 29
  Synthetic_PS8 HP,wherein nucleotides at positions 1-8 and 42-49 are modified with phosphorothioate linkage

SEQ ID NO: 33 - DNA, 50 bp, Artificial Sequence; identical to SEQ ID NO: 29
  Synthetic_FPS HP, wherein all nucleotides are modified with phosphorothioate linkage

SEQ ID NO: 34 - RNA, 50 nt, Artificial Sequence
  Synthetic_2′-O-Me HP, wherein all nucleotides are modified with 2′-OMe
uauuucucauucccccaucuuuaccagacaguguuagggaaugagaaaua

SEQ ID NO: 35 - RNA, 20 nt, Artificial Sequence
  Synthetic_siRNA
ucgaaguacucagcguaagu

SEQ ID NO: 36 - DNA, 60 bp, Artificial Sequence
  Synthetic_DNA
tcgaagtactcagcgtaagttcgaagtactcagcgtaagttcgaagtactcagcgtaagt

SEQ ID NO: 37 - DNA, 60 bp, Artificial Sequence; identical to SEQ ID NO: 36
  Synthetic_PS, wherein all nucleotides are modified with phosphorothioate linkage

SEQ ID NO: 38 - RNA, 60 nt, Artificial Sequence
  Synthetic_2′-O-Me, wherein all nucleotides are modified with 2′-OMe
ucgaaguacucagcguaaguucgaaguacucagcguaaguucgaaguacucagcguaagu

SEQ ID NO: 39 - DNA, 50 bp, Artificial Sequence
  Synthetic_HP_PKR(3)_miR141
tagctgtacttcaaccatctttaccagacagtgttattgaagtacagcta

SEQ ID NO: 40 - DNA, 50 bp, Artificial Sequence
  Synthetic_2SD HP_PKR(3)_ miR141, wherein all nucleotides are modified with phosphorothioate linkage
tagctgtacttcaagacagtgttattgacagtgttattgaagtacagcta

SEQ ID NO: 41 - RNA, 66 nt, Artificial Sequence
  Synthetic_GFP(1)_141_141, wherein all nucleotides are modified with 2′-OMe
ccaucuuuaccucacaguguuaccaucuuuaccucacaguguuauuauguuucagguucaggggga

SEQ ID NO: 42 - RNA, 64 nt, Artificial Sequence
  Synthetic_GFP(2)_141_141, wherein all nucleotides are modified with 2′-OMe
ccaucuuuaccucacaguguuaccaucuuuaccucacaguguuaaaauuugugaugcuauugcu

SEQ ID NO: 43 - RNA, 19 nt, Artificial Sequence
  Synthetic_siLuc
ucgaaguacucagcguaag

SEQ ID NO: 44 - RNA, 19 nt, Artificial Sequence
  Synthetic_siGFP
uuauguuucagguucaggg

SEQ ID NO: 45 - RNA, 63 nt, Artificial Sequence
  Synthetic_PKR(1)_141_141, wherein all nucleotides are modified with 2′-OMe
ccaucuuuaccucacaguguuaccaucuuuaccucacaguguuauaugugaggcagagaacga

SEQ ID NO: 46 - RNA, 63 nt, Artificial Sequence
  Synthetic_PKR(2)_141_141, wherein all nucleotides are modified with 2′-OMe
ccaucuuuaccucacaguguuaccaucuuuaccucacaguguuauauuucucauucccuuccu

SEQ ID NO: 47 - RNA, 64 nt, Artificial Sequence
  Synthetic_PKR(1)_200c_200c, wherein all nucleotides are modified with 2′-OMe
ccaucauuaccgcgcaguauuauccaucauuaccgcgcaguauuauaugugaggcagagaacga

SEQ ID NO: 48 - RNA, 65 nt, Artificial Sequence
  Synthetic_PKR(2)_200c_200c,wherein all nucleotides are modified with 2′-OMe
uccaucauuaccgcgcaguauuauccaucauuaccgcgcaguauuauauuucucauucccuuccu

SEQ ID NO: 49 - RNA, 57 nt, Artificial Sequence
  Synthetic_Luc_Luc_Luc,wherein all nucleotides are modified with 2′-OMe
ucgaaguacucagcguaagucgaaguacucagcguaagucgaaguacucagcguaag

SEQ ID NO: 50 - RNA, 63 nt, Artificial Sequence
  Synthetic_Luc_141_141,wherein all nucleotides are modified with 2′-OMe
ccaucuuuaccucacaguguuaccaucuuuaccucacaguguuaucgaaguacucagcguaag

SEQ ID NO: 51 - RNA, 65 nt, Artificial Sequence
  Synthetic_Luc_200c_200c, wherein all nucleotides are modified with 2′-OMe
uccaucauuaccgcgcaguauuauccaucauuaccgcgcaguauuaucgaaguacucagcguaag

SEQ ID NO: 52 - RNA, 50 nt, Artificial Sequence
  Synthetic_HP_PKR(3)_141, wherein all nucleotides are modified with 2′-OMe
uugaaguacagcuaccaucuuuaccucacaguguuauagcuguacuucaa

SEQ ID NO: 53 - RNA, 22 nt, Artificial Sequence
  Synthetic_miR-141*
ccaucuuuaccagacaguguua

SEQ ID NO: 54 - DNA, 50 bp, Artificial Sequence; identical to SEQ ID NO: 29
  Synthetic_HP_PKR(2)_141, wherein all nucleotides are modified with phosphorothioate linkage and 3′ biotin

SEQ ID NO: 55 - DNA, 50 bp, Artificial Sequence; identical to SEQ ID NO: 39
  Synthetic_HP_PKR(3)_141, wherein all nucleotides are modified with phosphorothioate linkage and 3′ biotin

SEQ ID NO: 56 - DNA, 50 bp, Artificial Sequence
  Synthetic_HP_Luc-141, wherein all nucleotides are modified with phosphorothioate linkage and 3′ biotin
tcgaagtactcagcccatctttaccagacagtgttagctgagtacttcga

SEQ ID NO: 57 - RNA, 19 nt, Artificial Sequence
  Synthetic_siAGO1
ggaguuacuuucauagcau

SEQ ID NO: 58 - RNA, 19 nt, Artificial Sequence
  Synthetic_siAGO2-1
gcacggaaguccaucugaa

SEQ ID NO: 59 - RNA, 19 nt, Artificial Sequence
  Synthetic_siAGO2-2
gcaggacaaagauguauua

SEQ ID NO: 60 - RNA, 19 nt, Artificial Sequence
  Synthetic_siAGO2-3
gggucuguggugauaaaua

SEQ ID NO: 61 - RNA, 19 nt, Artificial Sequence
  Synthetic_siAGO2-4
guaugagaacccaauguca

SEQ ID NO: 62 - RNA, 19 nt, Artificial Sequence
  Synthetic_siBcl-2
aucaaucuucagcacucuc

SEQ ID NO: 63 - RNA, 19 nt, Artificial Sequence
  Synthetic_siBcl-xL
uuggucccucaguaugguc

SEQ ID NO: 64 - RNA, 19 nt, Artificial Sequence
  Synthetic_siMcl-1
aaauucgauacuuccuucg

SEQ ID NO: 65 - RNA, 58 nt, Artificial Sequence
  Synthetic_Mcl-1_141_141, wherein all nucleotides are modified with 2′-OMe
ccaucuuuaccucacaguguuaccaucuuuaccucacaguguuaccgaacuacguagc

SEQ ID NO: 66 - RNA, 60 nt, Artificial Sequence
  Synthetic_Mcl-1_200c_200c, wherein all nucleotides are modified with 2′-OMe
uccaucauuaccgcgcaguauuauccaucauuaccgcgcaguauuaccgaacuacguagc

SEQ ID NO: 67 - RNA, 50 nt, Artificial Sequence
  Synthetic_HP_Mcl-1(1)_141, wherein all nucleotides are modified with 2′-OMe
gcuacguaguucggccaucuuuaccagacaguguuaccgaacuacguagc

SEQ ID NO: 68 - RNA, 50 nt, Artificial Sequence
  Synthetic_HP_Mcl-1(3)_141, wherein all nucleotides are modified with 2′-OMe
cgaaggaaguaucgccaucuuuaccagacaguguuacgauacuuccuucg

SEQ ID NO: 69 - RNA, 64 nt, Artificial Sequence
  Synthetic_Cy5.5_PKR-155_155,whereinall nucleotides are modified with 2′-OMe and 5′ Cy5
uuauguuucagguucagggggauuauguuuccagguucagggggauaugugaggcagagaacga

SEQ ID NO: 70 - RNA, 52 nt, Artificial Sequence
  Synthetic_HP_PKR(2)_155, wherein all nucleotides are modified with 2′-OMe
uauuucucauucccaaccccuaucacgauuagcauuaagggaaugagaaaua

SEQ ID NO: 71 - RNA, 52 nt, Artificial Sequence
  Synthetic_HP_PKR(3)_155, wherein all nucleotides are modified with 2′-OMe
uagcuguacuucaaaaccccuaucacgauuagcauuaauugaaguacagcua

SEQ ID NO: 72 - RNA, 24 nt, Artificial Sequence
  Synthetic_miR-155
uuaaugcuaaucgugauagggguu

SEQ ID NO: 73 - RNA, 40 nt, Artificial Sequence
  Synthetic_PKR mRNA (2)
caauaaugggaaggaagggaaugagaaauauuaaauucug

SEQ ID NO: 74 - RNA, 39 nt, Artificial Sequence
  Synthetic_PKR mRNA (3)
guauugaaaacaauugaaguacagcuaaauguaauaacg

SEQ ID NO: 75 - RNA, 63 nt, Artificial Sequence
  Synthetic_PKR(1)-155-155, wherein all nucleotides are modified with 2′-OMe
uuauguuucagguucagggggauuauguuucagguucagggggauaugugaggcagagaacga

SEQ ID NO: 76 - RNA, 63 nt, Artificial Sequence
  Synthetic_PKR(2)-155-155, wherein all nucleotides are modified with 2′-OMe
uuauguuucagguucagggggauuauguuucagguucagggggauauuucucauucccuuccu

SEQ ID NO: 77 - RNA, 63 nt, Artificial Sequence
  Synthetic_luc_155-155,wherein all nucleotides are modified with 2′-OMe
uuauguuucagguucagggggauuauguuucagguucagggggaucgaaguacucagcguaag

SEQ ID NO: 78 - DNA, 23 bp, Artificial Sequence
  Synthetic_U6_RT Primer
cgcttcacgaatttgcgtgtcat

SEQ ID NO: 79 - DNA, 25 bp, Artificial Sequence
  Synthetic_U6_Forward Primer
gcttcggcagcacatatactaaaat

SEQ ID NO: 80 - DNA, 23 bp, Artificial Sequence
  Synthetic_U6_Reverse Primer
cgcttcacgaatttgcgtgtcat

SEQ ID NO: 81 - DNA, 69 bp, Artificial Sequence
  Synthetic_miR-9_RT Primer
gaaagaaggcgaggagcagatcgaggaagaagacggaagaatgtgcgtctcgccttctttctcatacag

SEQ ID NO: 82 - DNA, 21 bp, Artificial Sequence
  Synthetic_miR-9_Forward Primer
gcgtctttggttatctagctg

SEQ ID NO: 83 - DNA, 21 bp, Artificial Sequence
  Synthetic_miR-9_Reverse Primer
cgaggaagaagacggaagaat

SEQ ID NO: 84 - DNA, 69 bp, Artificial Sequence
  Synthetic_miR-10b_RT Primer
gaaagaaggcgaggagcagatcgaggaagaagacggaagaatgtgcgtctcgccttctttccacaaatt

SEQ ID NO: 85 - DNA, 20 bp, Artificial Sequence
  Synthetic_miR-10b_Forward Primer
gctaccctgtagaaccgaat

SEQ ID NO: 86 - DNA, 21 bp, Artificial Sequence
  Synthetic_miR-10b_Reverse Primer
cgaggaagaagacggaagaat

SEQ ID NO: 87 - DNA, 69 bp, Artificial Sequence
  Synthetic_miR-17_RT Primer
gaaagaaggcgaggagcagatcgaggaagaagacggaagaatgtgcgtctcgccttctttcctacctgc

SEQ ID NO: 88 - DNA, 20 bp, Artificial Sequence
  Synthetic_miR-17_Forward Primer
gccaaagtgcttacagtgca

SEQ ID NO: 89 - DNA, 21 bp, Artificial Sequence
  Synthetic_miR-17_Reverse Primer
cgaggaagaagacggaagaat

SEQ ID NO: 90 - DNA, 69 bp, Artificial Sequence
  Synthetic_miR-22_RT Primer
gaaagaaggcgaggagcagatcgaggaagaagacggaagaatgtgcgtctcgccttctttcacagttct

SEQ ID NO: 91 - DNA, 20 bp, Artificial Sequence
  Synthetic_miR-22_Forward Primer
gcaagctgccagttgaagaa

SEQ ID NO: 92 - DNA, 21 bp, Artificial Sequence
  Synthetic_miR-22_Reverse Primer
cgaggaagaagacggaagaat

SEQ ID NO: 93 - DNA, 69 bp, Artificial Sequence
  Synthetic_miR-125b_RT Primer
gaaagaaggcgaggagcagatcgaggaagaagacggaagaatgtgcgtctcgccttctttctcacaagt

SEQ ID NO: 94 - DNA, 21 bp, Artificial Sequence
  Synthetic_miR-125b_Forward Primer
gcgtcgtaccgtgagtaataa

SEQ ID NO: 95 - DNA, 21 bp, Artificial Sequence
  Synthetic_miR-125b_Reverse Primer
cgaggaagaagacggaagaat

SEQ ID NO: 96 - DNA, 69 bp, Artificial Sequence
  Synthetic_miR-126_RT Primer
gaaagaaggcgaggagcagatcgaggaagaagacggaagaatgtgcgtctcgccttctttccgcattat

SEQ ID NO: 97 - DNA, 19 bp, Artificial Sequence
  Synthetic_miR-126_Forward Primer
gtccctgagaccctaactt

SEQ ID NO: 98 - DNA, 21 bp, Artificial Sequence
  Synthetic_miR-126_Reverse Primer
cgaggaagaagacggaagaat

SEQ ID NO: 99 - DNA, 69 bp, Artificial Sequence
  Synthetic_miR-155_RT Primer
gaaagaaggcgaggagcagatcgaggaagaagacggaagaatgtgcgtctcgccttctttcaaccccta

SEQ ID NO: 100 - DNA, 22 bp, Artificial Sequence
  Synthetic_miR-155_Forward Primer
gcggttaatgctaatcgtgata

SEQ ID NO: 101 - DNA, 21 bp, Artificial Sequence
  Synthetic_miR-155_Reverse Primer
cgaggaagaagacggaagaat

SEQ ID NO: 102 - RNA, 62 nt, Artificial Sequence
  Synthetic_Bcl-2(1)_155_155,wherein all nucleotides are modified with 2′-OMe
uuauguuucagguucagggggauuauguuucagguucagggggauugccugaagacuguuaa

SEQ ID NO: 103 - RNA, 62 nt, Artificial Sequence
  Synthetic_Bcl-2(2)_155_155,wherein all nucleotides are modified with 2′-OMe
uuauguuucagguucagggggauuauguuucagguucagggggaaugccacagaguuauucc

SEQ ID NO: 104 - RNA, 62 nt, Artificial Sequence
  Synthetic_Bcl-2(3)_155_155,wherein all nucleotides are modified with 2′-OMe
uuauguuucagguucagggggauuauguuucagguucagggggaauacacuauuugugagca

SEQ ID NO: 105 - RNA, 58 nt, Artificial Sequence
  Synthetic_Mcl-1_155_155, wherein all nucleotides are modified with 2′-OMe
uuauguuucagguucagggggauuauguuucagguucagggggaccgaacuacguagc

SEQ ID NO: 106 - RNA, 61 nt, Artificial Sequence
  Synthetic_bcl-xL(1)_222_222,whereinall nucleotides are modified with 2′-OMe
acccaguagccagauguagcuacccaguagccagauguagcuucuccuuccugcccuuccu

SEQ ID NO: 107 - RNA, 59 nt, Artificial Sequence
  Synthetic_bcl-xL(2)_222_222,whereinall nucleotides are modified with 2′-OMe
acccaguagccagauguagcuacccaguagccagauguagcuuaccugccagccuccuu

SEQ ID NO: 108 - RNA, 61 nt, Artificial Sequence
  Synthetic_luc_222-222,wherein all nucleotides are modified with 2′-OMe
acccaguagccagauguagcuacccaguagccagauguagcuucgaaguacucagcguaag

SEQ ID NO: 109 - RNA, 56 nt, Artificial Sequence
  Synthetic_Mcl-1_222_222, wherein all nucleotides are modified with 2′-OMe
acccaguagccagauguagcuacccaguagccagauguagcuccgaacuacguagc

SEQ ID NO: 110 - RNA, 58 nt, Artificial Sequence; identical to SEQ ID NO: 65
  Synthetic_Mcl-1-141-141, wherein all nucleotides are modified with 2′-OMe

SEQ ID NO: 111 - RNA, 60 nt, Artificial Sequence; identical to SEQ ID NO: 66
  Synthetic_Mcl-1-200c-200c, wherein all nucleotides are modified with 2′-OMe

SEQ ID NO: 112 - RNA, 63 nt, Artificial Sequence
  Synthetic_luc_let7f_let7f, wherein all nucleotides are modified with 2′-OMe
aacuauacaaugaacuaccucaaacuauacaaugaacuaccucaucgaaguacucagcguaag

SEQ ID NO: 113 - RNA, 58 nt, Artificial Sequence
  Synthetic_Mcl-1_let7f_let7f,whereinall nucleotides are modified with 2′-OMe
aacuauacaaugaacuaccucaaacuauacaaugaacuaccucaccgaacuacguagc

SEQ ID NO: 114 - RNA, 63 nt, Artificial Sequence
  Synthetic_bcl-xL(1)_let7f_let7f, wherein all nucleotides are modified with 2′-OMe
aacuauacaaugaacuaccucaaacuauacaaugaacuaccucaucuccuuccugcccuuccu

SEQ ID NO: 115 - RNA, 61 nt, Artificial Sequence
  Synthetic_bcl-xL(2)_let7f_let7f, wherein all nucleotides are modified with 2′-OMe
aacuauacaaugaacuaccucaaacuauacaaugaacuaccucauaccugccagccuccuu

SEQ ID NO: 116 - RNA, 63 nt, Artificial Sequence
  Synthetic_Bcl-xL(1)-141-141,whereinall nucleotides are modified with 2′-OMe
ccaucuuuaccucacaguguuaccaucuuuaccucacaguguuaucuccuuccugcccuuccu

SEQ ID NO: 117 - RNA, 61 nt, Artificial Sequence
  Synthetic_Bcl-xL(2)-141-141,whereinall nucleotides are modified with 2′-OMe
ccaucuuuaccucacaguguuaccaucuuuaccucacaguguuauaccugccagccuccuu

SEQ ID NO: 118 - RNA, 48 nt, Artificial Sequence
  Synthetic_HP_Luc_7f+222(10),whereinall nucleotides are modified with 2′-OMe
ucgaaguacucagcaacuauacaaacccaguagcgcugaguacuucga

SEQ ID NO: 119 - RNA, 71 nt, Artificial Sequence
  Synthetic_HP_Mcl-1_7f+222(full), wherein all nucleotides are modified with 2′-OMe
ccgaacuacguagcaacuauacaaucuacuaccucaacccaguagccagauguagcugcuacguaguucgg

SEQ ID NO: 120 - RNA, 48 nt, Artificial Sequence
  Synthetic_HP_Mcl-1_7f+222(10), wherein all nucleotides are modified with 2′-OMe
ccgaacuacguagcaacuauacaaacccaguagcgcuacguaguucgg

SEQ ID NO: 121 - RNA, 50 nt, Artificial Sequence
  Synthetic_HP_luc_let7,wherein all nucleotides are modified with 2′-OMe
ucgaaguacucagcaacuauacaaugaacuaccucagcugaguacuucga

SEQ ID NO: 122 - RNA, 49 nt, Artificial Sequence
  Synthetic_HP_luc_222, wherein all nucleotides are modified with 2′-OMe
ucgaaguacucagcacccaguagcgugauguagcugcugaguacuucga

SEQ ID NO: 123 - RNA, 50 nt, Artificial Sequence
  Synthetic_HP_Mcl-1_let7, wherein all nucleotides are modified with 2′-OMe
ccgaacuacguagcaacuauacaaugaacuaccucagcuacguaguucgg

SEQ ID NO: 124 - RNA, 49 nt, Artificial Sequence
  Synthetic_HP_Mcl-1_222,whereinall nucleotides are modified with 2′-OMe
ccgaacuacguagcacccaguagcgugauguagcugcuacguaguucgg

SEQ ID NO: 125 - RNA, 49 nt, Artificial Sequence
  Synthetic_HP_bcl-xL(5)_222-1, wherein all nucleotides are modified with 2′-OMe
uuccugcccuuccuacccaguagcgugauguagcuaggaagggcaggaa

SEQ ID NO: 126 - RNA, 49 nt, Artificial Sequence
  Synthetic_HP_bcl-xL(6)_222-4, wherein all nucleotides are modified with 2′-OMe
uaccugccagccucacccaguagcgugauguagcugaggcuggcaggua

SEQ ID NO: 127 - RNA, 49 nt, Artificial Sequence
  Synthetic_HP_bcl-xL(5)_222-6, wherein all nucleotides are modified with 2′-OMe
ucuccuuccugcccacccaguagcgugauguagcugggcaggaaggaga

SEQ ID NO: 128 - RNA, 50 nt, Artificial Sequence
  Synthetic_HP_bcl-xL(6)_let7-1, wherein all nucleotides are modified with 2′-OMe
cugccagccuccuuaacuauacaaugaacuaccucaaaggaggcuggcag

SEQ ID NO: 129 - RNA, 50 nt, Artificial Sequence
  Synthetic_HP_bcl-xL(6)_let7-4, wherein all nucleotides are modified with 2′-OMe
uaccugccagccucaacuauacaaugaacuaccucagaggcuggcaggua

SEQ ID NO: 130 - DNA, 76 bp, Artificial Sequence
  Synthetic_DB_PKR_S20_141, wherein n is C12SPACER
  misc_feature (61): n is t
  misc_feature (62): n is t
ccccccgaatgagaaataccatctttaccagacagtgttatatttctcattcccttccttnnaaggaagggggggg

SEQ ID NO: 131 - RNA, 20 nt, Artificial Sequence
  Synthetic_Mcl-1 mRNA
gcaaguggcaagaggauuau

(19) United States
(12) Patent Application Publication
(10) Pub. No.: US 2023/0129651 A1
(43) Pub. Date: Apr. 27, 2023

(54) HAIRPIN STRUCTURE NUCLEIC ACID MOLECULES CAPABLE OF MODULATING TARGET GENE EXPRESSION AND USES THEREOF

(71) Applicant: KOREA ADVANCED INSTITUTE OF SCIENCE AND TECHNOLOGY, Daejeon (KR)

(72) Inventors: Hyun Gyu PARK, Daejeon (KR); Yoosik KIM, Daejeon (KR); HANSOL KIM, Daejeon (KR); Jayeon SONG, Daejeon (KR); JunHyeok YOON, Daejeon (KR)

(21) Appl. No.: 17/842,891

(22) Filed: Jun. 17, 2022

(30) Foreign Application Priority Data
Jun. 18, 2021 (KR) 10-2021-0079272

(51) Int. Cl. C12N 15/113; A61P 35/00

(52) U.S. Cl. CPC C12N 15/1135; A61P 35/00; C12N 2310/531; C12N 2310/31; C12N 2310/32

(57) ABSTRACT

This application pertains to a hairpin nucleic acid molecule capable of modulating expression of a target gene and a use thereof. A nucleic acid molecule according to an embodiment can modulate expression of a target gene in a specific manner for cells in which a miRNA hybridizable therewith is present, finding advantageous applications in compositions for regulating expression of a target gene or pharmaceutical compositions for treating diseases.

### INCORPORATION-BY-REFERENCE OF MATERIAL SUBMITTED ELECTRONICALLY

Incorporated by reference in its entirety herein is a computer-readable nucleotide/amino acid sequence listing submitted herewith and identified as follows: 36,864 byte ASCII (Text) file named “OPP20221663US_SEQ_revised.TXT”, created on Oct. 28, 2022.

### CROSS REFERENCE TO RELATED APPLICATIONS

This patent application claims the priority benefit of Korean Patent Application No. 10-2021-0079272, filed Jun. 18, 2021, the disclosure of which is hereby incorporated by reference herein in its entirety

### BACKGROUND OF THE INVENTION

### 1. Field of the Invention

This application relates to hairpin-structured nucleic acid molecules capable of modulating target gene expression and uses thereof

### 2. Description of the Related Art

A microRNA (miRNA) is a non-coding RNA molecule composed of about 20 to 25 nucleotides and is recorded in genomes of higher animal and plant cells. microRNAs are known to play a core role in modulating cellular metabolisms and functions including generation, growth, differentiation, and death of cells and inhibit gene expression at transcriptional and post-transcriptional stages.

From a genome, a primary microRNA (Pri-miRNA) is transcribed by RNA polymerase. The Pri-miRNA transcript is edited by a nuclear RNase, called Drosha to generate a precursor microRNA (pre-miRNA) which is about 70-80 nucleotides long with an RNA hairpin structure). The pre-miRNA is then exported from the nucleus to the cytoplasm by exportin protein, etc. In the cytoplasm, the pre-miRNA undergoes the second processing with dicer, another RNase, to generate a mature microRNA (miRNA) (The second processing step takes place in the cytoplasm wherein dicer, another RNase, cleaves near the hairpin loop to release a mature microRNA (mature miRNA) duplex). Only one strand in the double-stranded miRNA is selected and incorporated into the ribonucleoprotein complex RISC which is thus active. The sequence of the single-stranded miRNA acts as a template for RISC to recognize a target mRNA and regulate the expression of the target gene.

In recent years, it has been reported that miRNA acts as an important regulatory factor in various diseases such as cancer, cardiovascular disease, and autoimmune disease. Accordingly, disease therapeutics that inhibit miRNA itself are being developed, but in many cases, effects of treating diseases are not explicitly achieved just by inhibiting disease-related miRNA itself. In order to treat a disease more effectively and stably, the present inventors developed a novel nucleic acid molecule that can regulate the expression of a target gene in a manner specific for the site where a disease-related miRNA is present, with the miRNA being not complementary to the target gene.

### RELATED ART DOCUMENTS

### Patent Literature

Korean Patent No. 10-2020-0085801 A

### SUMMARY OF THE INVENTION

An aspect of the present disclosure provides a stem-loop structure nucleic acid molecule that is capable of binding to a miRNA and an mRNA of a target gene.

Another aspect of the present disclosure provides a composition containing the nucleic acid molecule for regulating expression of a target gene.

A further aspect of the present disclosure provides a method for regulating expression of a target gene, using the nucleic acid molecule.

Still another aspect of the present disclosure provides a pharmaceutical composition containing the nucleic acid molecule for prevention or treatment of cancer.

A still further aspect of the present disclosure provides a method for prevention or treatment of cancer, the method including a step of administering the nucleic acid molecule to a subject in need thereof.

### BRIEF DESCRIPTION OF THE DRAWINGS

FIG. 1 is an image of a PAGE gel showing whether hairpin miRNA triggers of various lengths bind to a miRNA.

FIG. 2 shows a miRNA-miRNA trigger-mRNA ternary structure predicted through NUPACK simulation. In the left panels of FIG. 2, formation of base pairs among a miRNA, a mRNA, a miRNA trigger is depicted while the right panel is a numerical representation of the left panel, Red bars in FIG. 2 indicate concentrations of the ternary structures formed at a thermodynamic equilibrium after an aqueous solution containing 100 nM of each of miRNA, mRNA trigger, and mRNA is incubated at 37° C., showing overwhelmingly predominant formation of the miRNA-miRNA trigger-mRNA ternary structure.

FIG. 3 shows results of examining whether the hairpin miRNA trigger binds to a miRNA and/or a target mRNA in the presence or absence of the miRNA and/or the target mRNA. In FIG. 3, a hairpin miRNA trigger (HP) is indicated by (A), a hybridized form of a miRNA trigger and a miRNA by (B) (the trigger may exist in an open form as a result of binding to miRNA), and an associated form of all of miRNA, target mRNA, and miRNA trigger by (C).

FIG. 4 shows results of examining whether the hairpin miRNA trigger capable of binding to various types of miRNAs (miR-21, miR-200a, miR-200b, and miR-200c) binds to the miRNAs and/or a target mRNA in the presence or absence of the miRNA and/or the target mRNA.

FIG. 5a is an image of a PAGE gel in which hairpin miRNA triggers with chemical modifications (LNA HP, PS4HP, PS8 HP, FPS HP, 2′-O-Me HP) or without chemical modifications (DNA HP) have been run after being mixed with a cell lysate or human serum, and FIG. 5b is a graph of numeralized sizes of the bands detected in the gel. In the hairpin miRNA trigger structures of FIG. 5a, the green part accounts for LNA-modified nucleotides, the red part for PS-modified nucleotides, and the blue part for 2′-OMe-modified nucleotides.

FIG. 6 shows results of a cytotoxicity assay of chemically modified miRNA triggers. In FIG. 6, NT stands for a non-transfected group.

FIG. 7a is a schematic diagram illustrating the mechanism of action in which a 2-seed hairpin miRNA trigger (2SD HP) having a loop simultaneously hybridizable with two miRNAs represses the expression of a target gene. FIG. 7b shows PKR expression levels measured after transfection of into the hairpin miRNA trigger (HP) and the 2SD HP into Hela-141 cells.

FIG. 8 is a graph of fluorescence intensity measured after transfection of the miRNA trigger (GFP_141_141) into GFP-expressing Hela-141 cells.

FIGS. 9a and 9b show expression levels of PKR mRNA after transfection of the linear miRNA triggers (PKR-141-141 and PKR-200c-200c) and the controls including the linear miRNA triggers (Luc-141-141 and PKR-200c-200c) and the probe (Luc-Luc-Luc) into Hela, Hela-141, Hela-200c, and MCF-7 cell lines. Specifically, FIG. 9a shows results obtained with the PS-modified linear miRNA triggers and the probe and FIG. 9b shows results obtained with the 2′-OMe-modified linear miRNA triggers and the probe.

FIG. 10 shows expression levels of PKR mRNA after transfection of the hairpin miRNA trigger (PKR-141) into Hela, Hela-141, and MCF-7 cell lines.

FIG. 11 illustrates the regulatory effect of the miRNA trigger on target gene expression according to miRNA existence. Specifically, FIG. 11 shows the action of the miRNA trigger according to miRNA existence in schematic diagrams (left panel) and expression levels of the target PKR mRNA measured after transfection of the hairpin miRNA trigger (PKR-141) into Hela cells in the presence or absence of miR141 and transfection of the hairpin miRNA trigger (PKR-141) into Hela-141 cells in the presence or absence of miR-141* (right panel).

FIGS. 12a and 12b shows results of an assay for examining whether the miRNA trigger binds to an mRNA of the target gene. Specifically, FIG. 12a shows schematic illustrations of an RNA pulldown assay for examining whether the miRNA trigger according to an embodiment binds to a target gene, and FIG. 12b shows PKR mRNA levels bound to the miRNA trigger complex separated using streptavidin-coated magnetic beads, relative to those before the separation using magnetic beads.

FIGS. 13a and. 13b show experimental data obtained after examining whether the activity of a miRNA trigger according to an embodiment is associated with AGO protein. Specifically, FIG. 13a is a schematic diagram illustrating the inhibition of miRNA trigger activity by siRNA-induced repression of AGO protein expression. FIG. 13b shows expression levels of PKR mRNA after transfection the hairpin miRNA trigger, together with siAGO1, siAGO2, or a combination thereof (siAGO1+siAGO2), into Hela-141 cells. In FIG. 13b, MM stands for an experimental group transfected with the hairpin miRNA trigger alone.

FIGS. 14a and 14b show results of an assay for selecting a target gene effective for inducing apoptosis of cancer cells. Specifically, FIG. 14a shows cell viability measured after transfection of siMcl-1, siBcl-xL, and/or siBcl-1 into the breast cancer cell line MCF-7 and FIG. 14b shows cell viability measured after transfection of siMcl-1, siBcl-xL, and/or siBcl-1 into the pancreatic cancer cell line PANC1. As a control, siLuc was used. FIG. 14c shows protein expression levels as measured by wester blotting after transfection of siBcl-2, siMcl-1, siBcl-xL, and siMcl-1+siBcl-xL into the breast cancer cell line MCF-7. As a control, siLuc was used. Upon co-transfection of siMcl-1 and siBcl-xL, a band responsible for the apoptotic marker cleaved PARP appeared intensively.

FIG. 15 shows that the miRNA trigger according to an embodiment is able to discriminate cell lines and regulate expression of the target gene. Specifically, cell viability was measured after transfection of the linear miRNA triggers (Mcl-1-141-141 and Mcl-1-200c-200c) according to an embodiment into Hela, Hela-141, and Hela-200c cells.

FIG. 16 shows that the miRNA trigger (Mcl-1-141-141) according to an embodiment downregulated the protein expression of the target Mcl-1 in Hela-141 cells. In FIG. 16, Luc stands for Luc-141-141 and Mcl-1 represents a miRNA trigger (Mcl-1-141-141)-administered group.

FIG. 17 shows cell viability (%) measured after transfection of the hairpin miRNA triggers (Mcl-1(1)-141, Mcl-1(3)-141) according to an embodiment into Hela and Hela-141 cells. In FIG. 17, siMcl-1 was used as a positive control.

FIGS. 18a to 18f show results of in-vivo imaging performed days 1, 2, and 4 after injection of a fluorophore-conjugated linear miRNA trigger (5′-Cy5.5-PKR-miR-155-miR-155-3′) into mice.

FIGS. 19a and. 19b are FACS (FSC-A/SSC-A) profiles of blood cells extracted from mice injected with fluorophore-conjugated linear miRNA trigger (5′-Cy5.5-PKR-miR-155-miR-155-3′).

FIGS. 20a to 20c show the delivery of the miRNA triggers into peripheral blood cells as assayed by FACS.

FIGS. 21a to 21c show the delivery of the miRNA triggers into spleen-derived hematopoietic cells as assayed by FACS.

FIGS. 22a to 22c shows the delivery of the miRNA triggers into bone marrow hematopoietic cells as assayed by FACS.

FIG. 23 shows expression levels of miR-155 in the FLT3-ITD mutated cell lines MV4-11 and MOLM-14 and the controls FLT3-WT NB4 and HL60.

FIG. 24 shows results of an assay examining whether the hairpin miRNA triggers (2′-O-Me-PKR(2)_PM_S14_155 and 2′-O-Me-PKR(3)_PM_S14_155) according an embodiment bind to the miRNA and/or the mRNA.

FIG. 25 shows protein levels of the apoptotic marker and the Bcl-2 family measured after transfection of siRNAs siMcl-1, siBcl-2, and siBcl-xL) for the Bcl-2 family into blood cancer cells.

FIG. 26 shows expression levels of miR-155 after treatment of FLT3-ITD mutated AML cell lines (MV4-11 and NOIM-14) with AML therapeutic agents (cytarabine, azacitidine, decitabine, venetoclax, and gilteritinib). p-value <0.05, **: p-value <0.01, ***: p-value<0.001.

FIG. 27 shows expression levels of PKR mRNA measured after transfection of the linear miRNA trigger (PKR-155-155) according to an embodiment into MV-11 and HL60 cells.

FIG. 28 shows expression levels of various miRNAs (miR-9, miR-10b, miR-17, miR-22, miR-125b, miR-126, miR-155) in blood samples taken from the bone marrow of AML patients.

FIG. 29 shows PKR expression levels measured after injection of the linear miRNA trigger (PKR-155-155) according to an embodiment into 6528-sample taken from FLT3-WT patients and 6562-sample taken from FLT3-ITD mutated patients.

FIGS. 30a and. 30b show results after linear miRNA triggers capable of binding to miR-155 and various sites of Bcl-2 were transfected into AML cells. Specifically, FIG. 30a shows cell viability (%) measured after transfection of linear miRNA triggers according to an embodiment into MOLM-14, and FIG. 30b shows protein expression levels of Bcl-2 and an apoptotic marker (cleaved PARP) as measured by western blotting after transfection of linear miRNA triggers into MOLM-14 cells.

FIG. 31a shows cell viability measured after transfection of the Mcl-1-targeting linear miRNA (Mcl-1-155) into MV4-11 cell line and FIG. 31b shows cell viability measured after transfection of the Bcl-2-targeting linear miRNA (Mcl-1-155) into MV4-11 cell line.

FIGS. 32a and 32b show assay results after linear miRNA triggers capable of binding to miR-222 and various sites of Bcl-xL were transfected to breast cancer cells. Specifically, FIG. 32a shows cell viability (%) measured after transfection of the linear miRNA triggers according to an embodiment into MDA-MB-231 and FIG. 32b shows protein levels of Bcl-xL and an apoptotic marker (cleaved PARP) as measured by western blotting.

FIGS. 33a to 33c show assay results after linear miRNA triggers capable of binding to miR-222 and targeting Bcl-xL were transfected to breast cancer cells relatively low in miR-222 expression level. Specifically, FIG. 33a shows cell viability (%) measured after transfection of the linear miRNA triggers according to an embodiment into MDA-MB-453 and FIG. 33b shows protein expression levels of Bcl-xL and the apoptotic marker (cleaved PARP) as measured by western blotting.

FIG. 34 shows cell viability measured after linear miRNA triggers capable of binding to miR-141 or let7f and targeting Bcl-xL or Mcl-1 were transfected individually or in combination into the breast cancer cell line MCF-7.

FIG. 35 shows cell viability measured after linear miRNA triggers capable of binding to miR-222 or let7f and targeting Bcl-xL or Mcl-1 were transfected individually or in combination into the breast cancer cell line MDA-MB-231.

FIGS. 36 and. 37 show data for a hairpin miRNA trigger capable of binding to two types of miRNAs. Specifically, FIG. 36 is a schematic illustration for the mechanism in which a hairpin miRNA trigger capable of binding to two types of miRNAs is opened only in the presence of both the two types of miRNAs and thus can regulate a target gene. FIG. 37 shows cell viability measured after transfection of the hairpin miRNA trigger capable of binding to two types of miRNAs into MDA-MB-231 and MDA-MB-453 cells.

FIG. 38 shows degrees of apoptosis measured after hairpin miRNA triggers capable of binding to miR-222 and let-7f and targeting Mcl-1 or Bcl-xL were transfected individually or in combination into the breast cancer cell line MDA-MB-231.

FIG. 39a shows a dumbbell-shaped miRNA trigger in which a portion capable of binding mRNA is lengthened to a 20-nt size. FIG. 39b shows that when a dumbbell-shaped miRNA trigger (DB_HP) was incubated alone (lane 1) or in combination with an miRNA (lane 2), an mRNA (lane 3), and miRNA+mRNA (lane 4), the dumbbell-shaped trigger opened its structure and formed a ternary complex capable of a target mRNA.

FIG. 40 illustrates the mechanism of action and application strategy of the hairpin miRNA trigger.

### DETAILED DESCRIPTION OF THE INVENTION

Provided herein is a nucleic acid molecule that is capable of binding to both a miRNA and a target gene, thereby suggesting that the expression of the target gene can be regulated specifically in cells, tissues, and/or organs where the miRNA capable of binding to the nucleic acid molecule is present (and/or overexpressed).

The technique provided herein pertains to a nucleic acid molecule capable of regulating the expression of a target gene, and a use thereof and, more particularly, can regulate the expression of a target gene in a manner specific for the site where a miRNA is expressed (cells, tissues, and/or organs), wherein the target gene is not complementary to the nucleic acid sequence of the miRNA at all.

In the present disclosure, a nucleic acid molecule capable of binding to a miRNA and the mRNA of a target gene is endowed with a stem-loop structure whereby an improvement can be brought in the stability of the nucleic acid molecule and/or the potential of regulating the expression of the target gene in a manner specific for a miRNA-present environment. In an embodiment, the nucleic acid molecule retains a stem-loop structure in a miRNA-absent environment, but opens the stem-loop structure in a specific manner for a miRNA-present environment and as such, can bind to an mRNA of a target gene.

As used herein the term “nucleic acid” or “polynucleotide” refers to a polymer composed of deoxyribonucleotides, ribonucleotides, or modified nucleotides in a single- or double-stranded form and is intended to encompass nucleic acids bearing nucleotide analogs or modified backbone residues or linkages known in the art. For example, the term “nucleic acid” or “polynucleotide” includes single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases.

The term “miRNA” (microRNA), as used herein, refers to a small RNA that functions to mediate RNA interference (RNAi), and/or RNA silencing.

Herein, the term “nucleotide” is used as is recognized in the art. A nucleotide generally comprises a base, a sugar, and a phosphate moiety. The base may be a natural base (standard) or a modified base as is well known in the art. Such bases are generally located at the 1′ position of a nucleotide sugar moiety. Additionally, the nucleotides can be unmodified or modified at the sugar, phosphate and/or base moiety.

By “hybridizable” or “complementary” or “substantially complementary” as used herein, it is meant that a nucleic acid (e.g. RNA, DNA) comprises a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) [DNA, RNA]. In addition, for hybridization between two RNA molecules (e.g., dsRNA), guanine (G) base pairs with uracil (U).

For example, G/U base-pairing is partially responsible for the degeneracy (i.e., redundancy) of the genetic code in the context of tRNA anti-codon base-pairing with codons in mRNA. As used herein, the term “hybridization” refers to formation of a double-stranded nucleic acid between complementary single-stranded nucleic acids. Hybridization can occur when the complementarity between two nucleic acid strands is perfect (perfect match) or even when some mismatched residues exist. The complementarity level for hybridization may vary depending on hybridization conditions, particularly annealing temperature.

As used herein, the term “homology” describes a degree of consensus between nucleotide sequences or amino acid sequences given and can be expressed in percentage (%). For example, a homology can be determined using a readily available computer program in which sequence information, e.g., parameters, such as scores, identity, similarity, etc., are directly aligned between two polynucleotide molecules or two polypeptide molecules. The computer program may be BLAST (NCBI), CLC Main Workbench (CLC bio), MegAlign™ (DNASTAR Inc), etc.

By the wording (phrase “a nucleic acid molecule, an X region, a Y region, and/or a Y* region) “includes (comprises) a (specific) nucleic acid sequence (nucleotide sequence)”, it is meant that the nucleic acid molecule, the X region, the Y region, and/or the Y* region consists essentially of or includes the (specific) nucleotide sequence and can be construed to include a “substantially equivalent sequence” to the specific sequence, with a mutation (deletion, substitution, modification, and/or addition) imparted thereto, (or to not exclude the mutation).

As used herein, the term “stem-loop” structure refers to a structure including a double-stranded moiety (stem) formed by base-pairing through hydrogen bonding between sequences inside a single-stranded nucleic acid molecule, and an intervening single-stranded ring moiety (loop) with no intraloop hydrogen bonds. The term “stem-loop” may be used interchangeably with the term “hairpin” or “hairpin loop”. The nucleic acid molecule according to an embodiment has a stem-loop structure in an environment where a miRNA is absent or expressed at a low level, but becomes active (opens the hairpin structure) to regulate the expression of a target gene in an environment where a miRNA is present or overexpressed.

An aspect provides a nucleic acid molecule capable of binding to both a miRNA and an mRNA of a target gene, the nucleic acid molecule comprising:

an X region capable of binding to the miRNA;

a Y region capable of binding to the mRNA of the target gene; and

a Y* region capable of binding to the Y region

wherein the Y region and the Y* region complementarily match each other to form a stem-loop structure.

As used herein, the term “target gene” is a gene of which expression is inhibited at an mRNA level and/or a protein level by the nucleic acid molecule according to an embodiment of the present disclosure. The target gene may be an endogenous gene or a transgene introduced into a cell by an expression vector. For example, the target gene may be a gene which causes (or induce) a disease.

In an embodiment, the nucleic acid molecule may be represented by the following General Formula 1 or 2:

5′-Y region-X region-Y* region-3′ (General Formula 1)

5′-Y* region-X region-Y region-3′ (General Formula 2).

The term “bind” or “binding” means formation of a double-stranded nucleic acid as a result of hybridization between two nucleic acid strands complementary to each other whether perfect complementarity or some mismatch residues exist between the nucleic acid strands. In an environment where a miRNA is present or overexpressed (cells, tissues, and/or organs), the nucleic acid molecule according to an embodiment may include a double-stranded portion resulting from hybridization with the miRNA and a double-stranded portion resulting from hybridization with the mRNA of a target gene, thus forming the triple structure of miRNA-nucleic acid molecule-target mRNA in which the miRNA is associated with the target mRNA via the nucleic acid molecule.

The nucleic acid molecule may include an X region capable of binding to (hybridizing with) a miRNA. In an embodiment, the X region may include a nucleic acid sequence 60% or more, 65% or more, 70% or more, 75% or more, 80% or more, 85% or more, 90% or more, 92% or more, 94% or more, 95% or more, 96% or more, 97% or more, 98% or more, 98.5% or more, 99% or more, 99.5% or more, 99.8% or more, 99.9% or more, or 100% complementary to the entire or partial nucleic acid sequence of a miRNA of interest and as such, can bind to (hybridize) with the miRNA.

In an embodiment, the X region may be included in or include the loop structure of the nucleic acid molecule.

In an embodiment, the nucleic acid may comprise the X region in the range of 1 to 10, 2 to 10, 3 to 10, 1 to 8, 2 to 8, 3 to 8, 1 to 6, 2 to 6, 3 to 6, 1 to 5, 2 to 5, 3 to 5, 1 to 4, 2 to 4, or 3 to 4.

In an embodiment, the X region may include a plurality of Xm regions. Each of the Xm regions means a part capable of binding to one miRNA. For example, the X region may include X₁, X₂, . . . , and Xₙ (Xₘ may be X₁, X₂, . . . , or Xₙ). In this regard, the nucleic acid molecule can be represented by the following General Formula 3 or 4:

5′-Y region-X₁-X₂- . . . -Xₙ-Y* region-3′ (General Formula 3)

5′-Y* region-X₁-X₂- . . . -Xₙ-Y region-3′ (General Formula 4)

wherein X₁, X₂, . . . , and Xₙ are each capable of binding to one miRNA, and n and m are each a natural integer selected from, for example, 1 to 10 (1, 2, 3, 4, 5, 6, 7, 8, 9, or 10).

In an embodiment, when the nucleic acid molecule includes a plurality of Xm regions, the Xm regions may bind to the same or different miRNAs and may be composed of the same or different nucleic acid sequences.

In an embodiment, when the nucleic acid molecule includes a plurality of Xm regions, the nucleic acid molecule may further include a spacer (e.g., a nucleic acid sequence such as T, A, TT, AA, TTT, and/or AAA) between the different individual Xm regions.

In an embodiment, the nucleic acid molecule may include a plurality of Xm regions capable of binding to different respective miRNAs. The nucleic acid molecule including a plurality of Xm regions (for example 2 to 4 Xm regions) capable of binding to different respective miRNAs is activated (i.e., opens the hairpin structure) and thus can regulate the expression of a target mRNA only in an environment where all the different miRNAs exist.

In an embodiment, the X region or the Xₘ region may consist of a number of nucleotides the same as or smaller than that of the miRNA to which the nucleic acid molecule can bind. By way of example, the X region or the Xₘ region may be 15 to 30 nt, 15 to 28 nt, 15 to 27 nt, 15 to 26 nt, 15 to 25 nt, 16 to 30 nt, 16 to 28 nt, 16 to 27 nt, 16 to 26 nt, 16 to 25 nt, 17 to 30 nt, 17 to 28 nt, 17 to 27 nt, 17 to 26 nt, 17 to 25 nt, 18 to 30 nt, 18 to 28 nt, 18 to 27 nt, 18 to 26 nt, 18 to 25 nt, 19 to 30 nt, 19 to 28 nt, 19 to 27 nt, 19 to 26 nt, 19 to 25 nt, 20 to 30 nt, 20 to 28 nt, 20 to 27 nt, 20 to 26 nt, or 20 to 25 nt long.

In an embodiment, nucleotide residues of positions 10 and 11 from the 3′ end of the X region or the Xm region may be not complementary to the corresponding miRNA.

The nucleic acid molecule may include a Y region capable of binding to (hybridizing with) an mRNA of a target gene. In an embodiment, the Y region may include a nucleic acid sequence 60% or more, 65% or more, 70% or more, 75% or more, 80% or more, 85% or more, 90% or more, 92% or more, 94% or more, 95% or more, 96% or more, 97% or more, 98% or more, 98.5% or more, 99% or more, 99.5% or more, 99.8% or more, 99.9% or more, or 100% complementary to the entire or a partial nucleic acid sequence of mRNA of a target gene of interest so that it can bind to (hybridize with) the mRNA of the target gene.

In an embodiment, the Y region may include a nucleic acid sequence capable of binding to (hybridizing with) a partial nucleic acid of a target gene, for example, include a nucleic acid sequence complementary to a partial sequence of a 3′-untranslated region (UTR) of a target gene.

In an embodiment, the Y region may include a nucleic acid sequence capable of binding to (hybridizing with) a partial nucleic acid sequence of a target gene, for example, may include a nucleic acid sequence complementary to partial sequences of a 3′-untranslated region (UTR) and 5′-UTR of a target gene. In an embodiment, for the selection of a target region, a list of siRNA candidates for the target gene are acquired using on-line siRNA design tools (e.g., GenScript, Eurofins Genomics, siRNA Wizard, Block-iT RNAi Designer, etc.) and RNA-binding protein recognition regions and proximal regions (e.g., within about 20 nucleotides) obtained from such tools by ENCODE eCLIP data analysis can be used.

In an embodiment, the sequence of the Y region may be as long as or shorter than that of the mRNA to which the nucleic acid molecular can bind, for example, may be 5 to 30 nt, 5 to 28 nt, 5 to 25 nt, 5 to 23 nt, to 20 nt, 5 to 18 nt, 5 to 16 nt, 5 to 15 nt, 5 to 14 nt, 5 to 12 nt, 6 to 30 nt, 6 to 28 nt, 6 to 25 nt, 6 to 23 nt, 6 to 20 nt, 6 to 18 nt, 6 to 16 nt, 6 to 15 nt, 6 to 14 nt, 6 to 12 nt, 7 to 30 nt, 7 to 28 nt, 7 to 25 nt, 7 to 23 nt, 7 to 20 nt, 7 to 18 nt, 7 to 16 nt, 7 to 15 nt, 7 to 14 nt, 7 to 12 nt, 8 to 30 nt, 8 to 28 nt, 8 to 25 nt, 8 to 23 nt, 8 to 20 nt, 8 to 18 nt, 8 to 16 nt, 8 to 15 nt, 8 to 14 nt, 8 to 12 nt, 9 to 30 nt, 9 to 28 nt, 9 to 25 nt, 9 to 23 nt, 9 to 20 nt, 9 to 18 nt, 9 to 16 nt, 9 to 15 nt, 9 to 14 nt, 9 to 12 nt, 10 to 30 nt, 10 to 28 nt, 10 to 25 nt, 10 to 23 nt, 10 to 20 nt, 10 to 18 nt, 10 to 16 nt, 10 to 15 nt, 10 to 14 nt, 10 to 12 nt, 12 to 30 nt, 12 to 28 nt, 12 to 25 nt, 12 to 23 nt, 12 to 20 nt, 12 to 18 nt, 12 to 16 nt, 12 to 15 nt, or 12 to 14 nt long.

The nucleic acid molecule may include a Y* region capable of binding to (hybridizing with) the Y region. In an embodiment, the Y* region may include a nucleic acid sequence 60% or more, 65% or more, 70% or more, 75% or more, 80% or more, 85% or more, 90% or more, 92% or more, 94% or more, 95% or more, 96% or more, 97% or more, 98% or more, 98.5% or more, 99% or more, 99.5% or more, 99.8% or more, 99.9% or more, or 100% complementary to the entire nucleic acid sequence or a partial nucleic acid sequence of the Y region, so that the Y* can bind to (hybridize with) the Y region.

In an embodiment, the Y region and the Y* region include respective sequences complementary to each other and thus can hybridize with each other by base pairing through hydrogen bonds therebetween, forming a stem in a double-stranded structure. In an embodiment, the Y region and y* region are responsible for a stem structure and may include complementary sequences to each other in an anti-parallel manner (e.g., a 5′−0.3′ sequence of the Y region and a 3′−0.5′ sequence of the Y″ region are complementary to each other).

The stem structure may include a double-stranded nucleic acid sequence with a blunt end, or a 1 to 5 nt-long protrusion (overhang) at the 3′ end, 5′ end, or both ends. A protrusion within the length range is less prone to strand displacement of mRNA than a protrusion exceeding the upper limit of the range, thereby allowing the nucleic acid molecule to exhibit an excellent effect.

As used herein, the term “protrusion” or “overhang” refers to the terminal portion of the nucleotide sequence that is not base paired between the two strands of a double-stranded nucleic acid molecule.

The nucleic acid molecule may have a stem-loop structure and include a portion capable of binding to miRNA. In an embodiment, the nucleic acid molecule is adapted to dissociate the Y region from the Y* region when bound to miRNA. In detail, under the environment of miRNA existence (or miRNA overexpression), miRNA binds the X region to break the complementary bonding between the Y region and the Y* region so that the Y region thus dissociated from the Y* region can bind to an mRNA of a target gene.

In an embodiment, the nucleic acid molecule may bear at least one modified nucleotide or may include a backbone modification. The modified nucleotide may be a nucleotide residue modified with at least one selected from the group consisting of 2′-O-methyl, 2′-methoxyethoxy, 2′-fluoro, 2′-allyl, 2′-O-[2-(methylamino)-2-oxoethyl], 4′-thio, 4′-CH₂—O-2′-bridge, 4′-(CH₂) 2-O-2′-bridge, 2′-LNA, 2′-amino, and 2′-O—(N-methylcarbamate).

The backbone modification may be made by at least one selected from the group consisting of phosphonate, phosphorothioate, and phosphotriester.

The nucleic acid molecule according to an embodiment can bind to a miRNA wherein the miRNA may be a posttranslational regulatory, non-coding RNA that functions in post-transcriptional regulation by binding to a 3′UTR (untranslated region) of a target RNA (a target of miRNA) to promote mRNA degradation or to induce translational repression.

In an embodiment, the term “miRNA” refers to a mature miRNA, functioning as a guide RNA, which is formed by cleaving a precursor having a hairpin structure, called pre-miRNA (precursor microRNA), with the RNase III dicer.

In an embodiment, a miRNA may be coupled with an RISC (RNA-induced silencing complex) (or loaded into an RISC) to form a ribonucleotide complex. The nucleic acid molecule according to an embodiment may bind to both a miRNA and an mRNA of a target gene and the miRNA may be associated with an RISC which may, in turn, induce degradation (cleavage) and/or translational repression of the mRNA bound to the nucleic acid molecule.

In an embodiment, the miRNA may be present or overexpressed in diseased cells (tissues or organs) and may be absent or hypoexpressed in normal cells (tissues, or organs). The disease may be cancer and the diseased cells may be cancer cells.

The phrase “miRNA is present in diseased cells and is absent or hypoexpressed in normal cells” means that the miRNA is present or high in expression level (overexpressed) in diseased cells (abnormal cells) compared to normal cells and is absent or low in expression level in normal cells compared to diseased cells. Binding to a miRNA present or overexpressed in diseased cells, the nucleic acid molecule according to an embodiment can regulate (e.g., decrease) the expression of a target gene in a diseased cell-specific manner without any influence on the expression of the target gene in normal cells. As used herein, the term “normal cells” (tissue, or organs) refer to wild-type cells (tissues, or organs) and/or cells (tissues, or organs) isolated from a subject with no diseases (without the disease of interest).

The nucleic acid molecule according to the present disclosure may regulate expression of a target gene in a specific manner for cells (tissues, or organs) where a miRNA is present or overexpressed, for example, the nucleic acid molecule which bind to (i) a miRNA present in diseased cells and absent in normal cells and/or (ii) a miRNA higher in expression level in diseased cells than normal cells can regulate expression of a target gene in a diseased cell (tissue, or organ)-specific manner, and may have no influence on expression of the target gene in normal cells.

In an amount, the miRNA may be present or overexpressed cells (tissues, or organs), for example, may be at least one selected from the group consisting of miR-141, miR-21, miR-200c, miR-222, let-7f, miR-155, miR-24, miR-29a, miR-27 (e.g., miR-27a), miR-200a, miR-200b, miR-429, miR-205, miR-30a, miR-34, miR-203, miR-10b, miR-31, miR-9, miR-490, miR-29a, miR-204, miR-221, miR-138, miR-17, miR-19, miR-569, miR-9, miR-22, miR-29b, miR-125b, miR-126, miR-146a, miR-193a, miR-196b, miR-223, miR-492, miR-135b, miR-331, miR-374a, miR-519a, miR-191, miR-210, miR-24, miR-9, miR-103, and miR-107.

The nucleic acid molecule according to an embodiment may regulate expression of a target gene. For example, the target gene may be at least one selected from the group consisting of an anti-apoptotic gene (pro-apoptotic gene), an oncogene, a protooncogene, and an epithelial-mesenchymal transition (EMT) promoting gene. The anti-apoptotic gene may be Mcl-1, Bcl-2, and/or Bcl-xL, the oncogene may be Raf, EGFR, c-Sis, and/or Ras, and the protooncogene may be Ras, CYCD, Her2, and/or Myc. The angiogenic gene may be Twist1, Snail1, SLUG, Zeb1, TCF4, and/or TCF3. In an embodiment, the target gene may be at least one selected from the group consisting of mcl-1, bcl-xL, bcl-2, Snail1, Twist1, SLUG, Zeb1, TCF4, TCF3, FLT3, STAT3, c-Sis, EGFR, Ras, CYCD, Her2, Myc, Raf, VIM, CDH2, FN1, ACTA2, COL1A1, and SNAI2.

Another aspect provides a composition for regulating expression of a target gene. The composition for regulating expression of a target gene contains the nucleic acid molecule according to an embodiment, and as such, can regulate expression of the target gene in a specific manner for cells, tissues, or organs where a specific miRNA exists.

In an embodiment, the composition for regulating expression of a target gene may downregulate mRNA and/or protein expression of the target gene.

The composition for regulating expression of a target gene may further contain a vehicle in addition to the nucleic acid molecule. The vehicle may be at least one selected from the group consisting of a lipid molecule, a liposome, a micelle, a cationic lipid, a protein particle, an inorganic nanoparticle, and/or a virus.

In one embodiment, the nucleic acid molecule may be delivered by direct application or in the form of a complex with a cationic lipid or the form of a package within a liposome. For example, the nucleic acid molecule can be administered to cells and/or a subject by a variety of methods known to those of skill in the art, including encapsulation in liposomes, by iontophoresis, or by incorporation into other vehicles, such as biodegradable polymers, hydrogels, cyclodextrins poly(lactic-co-glycolic)acid (PLGA) and PLCA microspheres, biodegradable nanocapsules, and bioadhesive microspheres.

As used herein, the term “liposome” refers to a vesicle composed of amphipathic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the nucleic acid molecule. The lipophilic material isolates the aqueous interior from an aqueous exterior, which typically does not include the nucleic acid molecule, although in some examples, it may. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the nucleic acid molecule are delivered into the cell where the nucleic acid molecule can specifically bind to a target RNA and can mediate RNAi. In some embodiments, the liposomes are also specifically targeted to direct the nucleic acid molecule to particular cell types.

“Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

Another aspect provides a pharmaceutical composition containing the nucleic acid molecule for prevention or treatment of cancer.

In an embodiment, the pharmaceutical composition may induce apoptosis of cancer cells or inhibit metastasis of cancer cells.

In an embodiment, the cancer may be:

at least one solid cancer selected from the group consisting of liver cancer, lung cancer (e.g., non-small cell lung cancer), pancreatic cancer, breast cancer, colon cancer, pancreatic cancer, ovarian cancer, endometrial cancer, cervical cancer, gallbladder cancer, gastric cancer, biliary tract cancer, colorectal cancer, head and neck cancer, thyroid cancer, brain tumor, malignant melanoma, prostate cancer, testicular cancer, and tongue cancer, or

at least one blood cancer selected from the group consisting of leukemias such as chronic lymphocytic leukemia (CLL), acute lymphocytic leukemia (ALL), non-Hodgkin lymphomas (NHL), acute myeloid leukemia (AML), and lymphomas.

The digestive disease may be at least one selected from the group consisting of Peptic Ulcer Disease, PUD, Gastroesophageal Reflux Disease, constipation, diarrhea, Constipation, Diarrhea and Irritable Bowel Syndrome, Nausea and Vomiting, Inflammatory bowel disease, Pancreatitis, Liver Cirrhosis and Complications, Viral Hepatitis, Drug-Induced Liver Injury, but not limited to thereto.

The kidney disease may be at least one selected from the group consisting of Fluid and Electrolyte Disorders, Acid-base Disorders, Drug-Induced Kidney Disease, Renal Impairment, Acute Kidney Injury, Chronic Kidney Disease, but not limited to thereto.

The nerve disease may be at least one selected from the group consisting of Headache, Epilepsy, Alzheimer's disease, Parkinson's disease, but not limited to thereto.

The mental disease may be at least one selected from the group consisting of Major Depressive Disorder, Schizophrenia, Generalized Anxiety Disorder, Panic Disorder, Bipolar Disorder, Attention Deficit Hyperactivity Disorder, Alcohol, Nicotine, and Caeine Addiction, Sleep Disorder, Eating disorders, but not limited to thereto.

The blood and tumor disease may be at least one selected from the group consisting of Anemias, Lung Cancer, Gastric Cancer, Colorectal Cancer, Breast Cancer, Gynecologic Cancers, Prostate Cancer, Leukemias, Lymphomas, but not limited to thereto.

The cardiovascular disease may be at least one selected from the group consisting of Hypertension, Heart Failure, Ischemic Heart Diseases, Acute Coronary Syndrome, ACS, Arrhythmias, Dyslipidemia, Stroke, Venous Thromboembolism, Peripheral Arterial Disease, Hypovolemic Shock, but not limited to thereto.

The respiratory disease may be at least one selected from the group consisting of Asthma, Chronic Obstructive Pulmonary Disease, Allergic Rhinitis, but not limited to thereto.

The endocrinopathy may be at least one selected from the group consisting of Diabetes Mellitus, Thyroid Disorder, Pituitary & Adrenal Gland Disorders, but not limited to thereto.

The infection disease may be at least one selected from the group consisting of Upper respiratory tract infection, Pneumonia, Urinary tract infection, Tuberculosis, Meningitis, Gastrointestinal/Intraabdominal Infections, Skin and Soft tissue Infection, Super&cial Fungal Infections/Deep mycoses, Sepsis, Sexually Transmitted Infection, STI, but not limited to thereto.

The muscloskeletal disease may be at least one selected from the group consisting of Osteoarthritis, Rheumatoid Arthritis, Osteoporosis, Gout and Hyperuricemia, but not limited to thereto.

The obstetrics and gynecology disease may be at least one selected from the group consisting of Drug Use in Pregnancy and Lactation, Menopause, Urinary Incontinence, but not limited to thereto.

The genito-urinary disease may be at least one selected from the group consisting of Benign Prostatic Hyperplasia, Prostatitis, but not limited to thereto.

The skin disease may be at least one selected from the group consisting of Atopic Dermatitis, and Psoriasis, but not limited to thereto.

The ophthalmologic disease may be Glaucoma, but not limited to thereto.

The pharmaceutical composition may be administered as an independent drug or may be co-administered with other drugs. In the case of co-treatment, they may be administered simultaneously or sequentially.

In an embodiment, the pharmaceutical composition may further contain an anticancer agent in addition to the nucleic acid molecule. The additionally contained anticancer agent may be an agent that is not designed to reduce the expression of the miRNA hybridizable with the nucleic acid molecule. For example, the pharmaceutical composition may further contain an anticancer agent selected from the group consisting of cytarabine, azacitidine, decitabine, paclitaxel, Adriamycin, and tamoxifen.

For practical application, the pharmaceutical composition may be formulated into oral dosage forms such as pulvis, granules, capsules, tablets, aqueous suspensions, and the like, agents for external use, suppositories, and sterile injections. An pharmaceutical composition according to an embodiment may include a pharmaceutically effective carrier. For oral administration, the pharmaceutically effective carrier may include a binder, a lubricant, a disintegrator, an excipient, a solubilizer, a dispersing agent, a stabilizer, a suspending agent, a coloring agent, and a perfume. For injectable formulation, the pharmaceutically effective carrier may include a buffering agent, a preserving agent, an analgesic, a solubilizer, an isotonic agent, and a stabilizer. For formulations of topical administration, the pharmaceutically effective carrier may include a base, an excipient, a lubricant, and a preservative. The pharmaceutical composition of the present disclosure may be formulated in various forms by adding the pharmaceutically effective carriers. For example, for oral administration, the pharmaceutical composition may be formulated into tablets, troches, capsules, elixirs, suspensions, syrups or wafers. For injectable preparations, the pharmaceutical composition may be prepared into a single-dose ampule or multidose container. The pharmaceutical composition may be formulated into single-dose ampule or multidose container. The pharmaceutical composition may be also formulated into solutions, suspensions, tablets, pills, capsules, and sustained release preparations.

Examples of carriers, excipients and diluents suitable for formulation include lactose, dextrose, sucrose, sorbitol, mannitol, xylitol, erythritol, maltitol, starch, acacia, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate, mineral oils or the like. In addition, the formulation may further include fillers, anti-coagulating agents, lubricants, humectants, perfumes, antiseptics or the like.

As used herein, the terms “pharmaceutically effective” means neither significantly stimulating an organism nor inhibiting the biological activity and characteristics of an active material administered. According to an embodiment, the pharmaceutical composition including a pharmaceutically effective carrier may be prepared into a formulation selected from the group consisting of a tablet, a pill, pulvis, granules, a capsule, a suspension, a liquid solution for internal use, an emulsion, a syrup, a sterile aqueous solution, a non-aqueous solution, a suspension, an emulsion, a lyophilizate, and a suppository.

The pharmaceutical composition may be in various formulations for oral or parenteral administration. The pharmaceutical composition may be formulated with a typically used diluent or excipient such as a filler, a thickener, a binder, a humectant, a disintegrant, a surfactant, etc.

Solid formulation agents for oral administration include tablets, pills, powders, granules, and capsules, and such solid dosage forms are formulated by mixing at least one active ingredient with one or more excipients, such as starch, calcium carbonate, sucrose, lactose, and gelatin. Also, lubricants such as magnesium stearate and talc can be used other than simple excipients. Liquid formulation agents for oral administration may be exemplified by suspensions, solutions for internal use, emulsions, and syrups, and may include various such as humectants, sweeteners, fragrances, and preservatives, in addition to simple diluents such as water and liquid paraffin.

Formulation agents for parenteral administration may be illustrated as sterile aqueous solutions, nonaqueous solvents, suspentions, emulsions, lyophilizates, and suppositories. Nonaqueous solvents and suspensions may include propylene glycol, polyethylene glycol, vegetable oil such as olive oil, and injectable esters such as ethyl oleate. As a base of suppositories, witepsol, macrogol, Tween 61, cacao fat, lanolin fat, or glycerogelatin may be used.

In an embodiment, the pharmaceutical composition may be administered to a subject via various routes. As used herein, the term “administration” means providing a given substance to a subject (patient) by any suitable method. The pharmaceutical composition may be administered orally or parenterally through any general route as long as it can reach the target tissue. All modes of administration may be contemplated. For example, oral administration or parenteral administration, such as intravenous, intramuscular, subcutaneous, intraperitoneal, intralesional, and topical administration, may be employed. In addition, the composition according to an embodiment may also be administered using any device capable of delivering the active ingredient to target cells.

The pharmaceutical composition according to the present disclosure may be administered through various routes, including, but not limited to, oral, intravenous, intramuscular, intraarterial, intramedullary, intradural, intracardial, transdermal, subcutaneous, intraperitoneal, intranasal, gastrointestinal, local, sublingual, and rectal routes. As used herein, the term “partenteral” is intended to encompass subcutaneous, intradermal, intravenous, intramuscular, intra-articular, intra-synovial, intrasternal, intrathecal, intralesional and intracranial injection or infusion techniques. The pharmaceutical composition of the present disclosure may also be administered in the form of suppositories for rectal administration.

The pharmaceutical composition of the present disclosure may vary depending on various factors, including the activity of a particular compound used, the patient's age, weight, general health, sex, diet, administration time, administration mode, excretion rate, drug combination and the severity of a particular disease to be prevented or treated. The dose of the pharmaceutical composition and/or the effective amount of the active ingredient (i.e., nucleic acid molecule) of the pharmaceutical composition may be suitably selected by a person skilled in the art and may vary depending on the patient's condition, weight, the severity of the disease, the type of drug, administration mode and period. The pharmaceutical composition may be administered at a dose of 0.0001 to 50 mg/kg or 0.001 to 50 mg/kg per day. The pharmaceutical composition may be administered once or several times (e.g., twice to four times, or three times) per day.

Another aspect provides a method for repressing expression of a target gene, the method including a step of administering the nucleic acid molecule, and/or the composition for regulating expression of the gene in an effective amount to a subject. The method for repressing expression of a target gene may further include a step of identifying whether the subject (individual) is in need of repressing expression of the gene, prior to the administering step. In an embodiment, the individual may include (transcribe) a miRNA hybridizable with the nucleic acid molecule.

In an embodiment, the method for repressing expression of a target gene may be to repress expression of a target gene in a target cell in vitro. The target cell may be a cell in which a miRNA hybridizable with the nucleic acid molecule exists or is overexpressed.

Another aspect provides a method for prevention or treatment of a cancer or a proliferative disease, the method including a step of administering the nucleic acid molecule, the composition for regulating expression of a target gene, and/or the pharmaceutical composition in a pharmaceutically effective amount to a subject. The prophylactic or therapeutic method may include identify whether the subject (individual) is in need of preventing or treating a cancer or a proliferative disease, prior to the administering step.

In the method for regulating expression of a gene or the method for prevention or treatment of a cancer or a proliferative disease, the method, route, and dose of the nucleic acid and/or the composition for repressing expression of a target gene are as defined above.

As used herein, the term “pharmaceutically effective amount” or “effective amount” refers to a content or dose of an active ingredient (nucleic acid molecule according to an embodiment) capable of showing desirable pharmacological effects (e.g., effects of inhibiting expression of a target gene or preventing and/or treating cancer or a proliferative disease) and it may be determined in a variety of ways, depending on factors such as formulation methods, administration modes, patient's age, body weight, sex, pathologic conditions, and diets, administration time, administration route, excretion speed, and reaction sensitivity.

The term “subject”, “individual”, or “patient”, as used herein, means an organism to which an nucleic acid molecule according to an embodiment can be administered. The subject may include mammals (e.g., primates such as humans, monkeys, or mammals exclusive of humans), donors or recipients of explanted cells, and cells or tissues isolated from the mammals, or a culture thereof.

In an embodiment, when the cancer to be prevented and/or treated by the pharmaceutical composition is breast cancer, the nucleic acid molecule contained in the pharmaceutical composition may bind to at least one miRNA selected from the group consisting of miR-222, miR-141, miR-21, miR-200c, miR-222, let-7f, miR-492, miR-135b, miR-331, miR-374a, miR-519a, miR-191, miR-210, and miR-24 and to at least one target gene selected from the group consisting of mcl-1, bcl-xL, and bcl-2.

In an embodiment, when the cancer to be prevented and/or treated by the pharmaceutical composition is acute myeloid leukemia (AML), the nucleic acid molecule contained in the pharmaceutical composition may bind to at least one miRNA selected from the group consisting of miR-155, miR-21, miR-9, miR-490, miR-29a, miR-204, miR-221, miR-138, miR-17, miR-19, miR-569, miR-9, miR-10b, miR-22, miR-29b, miR-125b, miR-126, miR-146a, miR-193a, miR-196b, and miR-223 and to at least one target gene consisting of mcl-1, bcl-xL, and bcl-2.

In an embodiment, when the cancer to be prevented and/or treated by the pharmaceutical composition is uterine cervical cancer, the nucleic acid molecule contained in the pharmaceutical composition may bind to at least one miRNA selected from the group consisting of miR-141 and miR-200c and to at least one target gene selected from the group consisting of mcl-1, bcl-xL, and bcl-2.

Being capable of regulating expression of a target gene in a specific manner for cells where a miRNA hybridizable with the nucleic acid molecule according to an embodiment, the nucleic acid molecule can find advantageous applications for use in a composition for regulating expression of a target gene or a pharmaceutical composition for treatment of a disease.

### DETAILED DESCRIPTION

A better understanding of the present disclosure may be obtained in light of following examples which are set forth to illustrate, but are not to be construed to limit, the present disclosure.

### Reference Example 1. Construction of miRNA Trigger

Polynucleotides (hereinafter referred to as “miRNA triggers”) that each include a portion capable of binding to a miRNA and a portion capable of binding to an mRNA were requested for synthesis in Bioneer and purified through HPLC before use in experiments. Intracellular stability was achieved by introducing a chemical modification such as LNA (locked nucleic acid), PS (phosphorothioate), or 2′-O-Me (2′-O-Methyl) into the polynucleotides upon construction thereof.

### Reference Example 1-1. Construction of miRNA Trigger in Linear Structure

The miRNA triggers constructed in this Example have the following structure:

[5′-portion capable of binding to miRNA (1)(mi(1)*)-portion capable of binding to miRNA (2)(mi(2)*)-portion capable of binding to mRNA of target gene (T*)-3′].

The portions capable of binding to miRNAs were as long as the miRNAs to be hybridized therewith and ranged in length from 20 to 25 nt depending on types of miRNAs.

The portions capable of binding to miRNAs were each designed to mismatch with the miRNA at the 10^{th} to 11th nucleotide residues from the 3′ end thereof and to include a sequence perfectly complementary to the miRNA, except for the mismatched residues.

The portion capable of binding to an mRNA of a target gene (hereinafter referred to as “target mRNA) was designed to be 14 to 20 nt in length and include a sequence complementary to that of the target mRNA.

The miRNA trigger constructed in this Example included two portions capable of two respective miRNAs and their sequences might be identical to or different from each other.

The miRNA trigger constructed in the Example was named “linear miRNA trigger” or “linear probe” (LP) to discriminate from the miRNA trigger constructed in the Example 1-2, below. Specific names of individual linear miRNA triggers were made in the pattern of [name of hybridizable target gene_name of hybridizable miRNA(2)_name of hybridizable miRNA(1)].

### Reference Example 1-2. Construction of miRNA Trigger in Hairpin Structure

The miRNA triggers constructed in this Example have the following structure:

[5′-portion capable of binding to mRNA of target gene (T*)-portion capable of binding to miRNA (mi*)-portion capable of binding to T* (T)-3′].

Since the miRNA trigger constructed in this Example included sequences complementarily hybridizable with each other (T and T* in the structure), the flank portions (T and T*) of the mi* portion are hybridized with each other so that the miRNA trigger may have the form of a hairpin miRNA trigger in a stem-loop structure. The stem structure in the hairpin miRNA trigger results from the hybridized structure between T and T* portions while the loop structure is accounted for by the mi* portion. The miRNA trigger having such a hairpin structure was named “hairpin miRNA trigger” or “hairpin probe” (HP). Specific names of individual hairpin miRNA triggers were made in the pattern of [HP_name of hybridizable target gene_name of hybridizable miRNA].

The portion capable of binding to a miRNA (mi*), (that is, the portion corresponding to the loop of the hairpin) was as long as the miRNA to be hybridized therewith and ranged in length from 20 to 25 nt depending on types of the miRNA. When the portion binding complementarily to a miRNA was designed to mismatch with the miRNA at the 10th to 11t nucleotide residues (M2) from the 3′ end thereof and to include a sequence perfectly complementary to the miRNA, except for the mismatched residues, specific names of individual hairpin miRNA triggers were made in the pattern of [HP_name of hybridizable target gene_M2_name of hybridizable miRNA]. When the portion capable of binding to a miRNA was designed to have no mismatches with the miRNA, the HP was named in the pattern of [HP_name of hybridizable target gene_name of hybridizable miRNA]. The portion capable of binding to an mRNA of a target gene (hereinafter referred to “target mRNA), that is, the stem of the hairpin, was designed to range in length from 12 to 22 nt and include a sequence complementary to that of the target mRNA.

### Reference Example 2. Design of miRNA Trigger and Assay for Performance Thereof

In order to examine whether intended hybridization was made among a miRNA trigger (M2_S12_miR141: 5′-CAAGAGGATTATCCATCTTTACCTCACAGTGTTAATAATCCTCTTG-3′; SEQ ID NO: 18), a miRNA (miR-141: 5′-UAACACUGUCUGGUAAAGAUGG-3′; SEQ ID NO: 17), and an mRNA (Mcl-1 mRNA 5′-GCAAGUGGCAAGAGGAUUAU-3′; SEQ ID NO: 131), a thermodynamic analysis was conducted using NUPACK (California Institute of Technology, http://www.nupack.org/). This analysis was carried out 37° C. in an aqueous solution containing 100 nM constituents (miRNA trigger, miRNA, mRNA) and 1.0 M Na⁺ ions. Using Oligo Analyzer version 3.1 of the IDT (Integrated DNA Technologies) website (http://www.idtdna.com), the melting point (Tm) was analyzed. The reaction products were separated at a constant voltage of 120 V for 180 minutes in a 20% polyacrylamide gel in 1×TBE buffer (Tris base 89 mM, boric acid 89 mM, EDTA 20 mM)). After staining with GelRed, a gel image was obtained using Gel Doc EZ Imager (Bio-rad, Hercules, USA).

### Reference Example 3. Assay for Resistance to Biological Degradation

A human serum was purchased from Sigma-Aldrich. A Hela cell lysate was prepared using RIPA buffer (NaCl 150 mM, EDTA 5 mM, Tris 50 mM, NP-40 1.0%, sodium deoxycholate 0.5%, SDS 0.1%). Oligonucleotides were mixed with the human serum, the cell lysate, or distilled water and incubated at 37° C. for 24 hours. Final concentrations of the human serum and the cell lysate were 80% and 5 mg/mL, respectively. Thereafter, the mixture was loaded to a centrifugal filter (MWCO=30 kDa, Millipore) and centrifuged at 14,000 g fir 30 minutes. Finally, a filtrate containing an oligonucleotide (˜15 kDa) was obtained. Separation was made for 120 minutes under a constant voltage of 120 V in a 15% polyacrylamide gel in 1×TBE buffer. After staining with EtBr, a gel image was obtained using Gel Doc EZ Imager (Bio-rad, Hercules, USA).

### Reference Example 4. Cell Culture and Establishment of Stable Cell Line

A total of 11 cancer cell lines (HeLa, HeLa-141, HeLa-200c, MCF-7, MDA-MB-231, MDA-MB-453, HL-60, MV4-11, NB-4, and MOLM-14) were cultured according to the recommended conditions required therefor, respectively. The cell lines were purchased from the American Type Culture Collection (ATCC; Manassas, Va., US). Genomic Hela-141 and Hela-200c cells were genetically modified by adenoviral genetic recombination. The transformed Hela cell lines were provided from the laboratory of Professor V. Narry Kim, Seoul National University. The tetracycline-off system was used to establish Hela-141 and Hela-200c that overexpressed miR-141 and miR-200c, respectively, compared to their parent cell line. Each cell line was maintained at 37° C. in a 5% CO₂ atmosphere and passaged every 3-4 days upon confluency. In all experiments, the cells were incubated and stabilized at least 18 hours before experimentation. All the cells used in this study were maintained in DMEM (Dulbecco's Modified Eagle Media) or RPMI-1640 supplemented with 10% fetal bovine serum (FBS).

### Reference Example 5. Transfection of miRNA Trigger

Transfection into adherent cells (HeLa, HeLa-141, HeLa-200c, MCF-7, MDA-MB-231, and MDA-MB-453) was performed using Lipofectamine 3000 (Thermo Fisher Scientific) according to the manufacturer's instruction. For transfection into suspended cells (HL-60, MV4-11, NB-4, and MOLM-14), the Neon transfection system (Thermo Fisher Scientific) was used according to the manufacturer's instruction.

### Reference Example 6. Assay for miRNA Trigger in AML

### Patient Sample

Peripheral blood samples were taken from seven AML patients. All patient information and samples were delivered from the Seoul National University Hospital (SNUH) in compliance with the Institutional Review Board (IRB) requirements for use of research samples. The cells were thawed and incubated at a density of 1×10⁶ cells/ml or greater in DMEM (Dulbecco's Modified Eagle Media) supplemented with 10% fetal bovine serum (FBS), sodium pyruvate, and L-glutamine (GIBCO; Grand Island, N.Y., US).

### Reference Example 7. Western Blot

miRNA triggers were transfected into cells in the same manner as in Reference Example 4. After 72 hours, the cells were collected using a cell scraper and lysed with a RIPA buffer (NaCl 150 mM, EDTA 5 mM, Tris 50 mM, NP-40 1.0%, sodium deoxycholate 0.5%, SDS 0.1%). The cell lysate was sonicated 15 times, each for 20 sec, with a halt for 30 sec every sonication. Following centrifugation at 5,000 g for 5 min, the supernatant was taken and used to extract proteins therefrom. A total of 30-40 μg of protein sample was separated in 10-15% SDS-PAGE gel and transferred onto a PVDF membrane through a semi-dry blotting system. The membrane was blocked for 1 hour in 5% skimmed milk and incubated for 2 hours with a 1,000-fold dilution of a primary antibody in, 1% skimmed milk. As the primary antibody, PKR ((#12297S, Cell Signaling Technology, MA, US), Mcl-1 (#5453S, Cell Signaling Technology, MA, US), Bcl-2 (#4223S, Cell Signaling Technology, MA, US), Bcl-xl (#2764S, Cell Signaling Technology, MA, US), or B-tublin (#2148 Cell Signaling Technology, MA, US) was used. The membrane incubated with the primary antibody was washed three times with 1×PBST buffer (NaCl 137 mM, KCl 2.7 mM, Na₂HPO₄ 10 mM, KH₂PO₄ 1.8 mM, Tween 20 0.1%) for 10 min each wash before incubation with a 1,000-fold dilution of a secondary antibody in 1% skimmed milk for 1 hour. The secondary antibody for western blotting was Goat Anti-Rabbit IgG (H+L)-HRP Conjugate (#1706515, Bio-Rad) or Goat Anti-Mouse IgG (H+L)-HRP Conjugate (#1706516, Bio-Rad). The membrane incubated with the secondary antibody was washed three times with 1×PBST for 10 min each wash, and then reacted with Clarity Western ECL Substrate (#1705061, Bio-Rad), after which images were obtained and analyzed using ChemiDoc (Bio-rad, Hercules, USA).

### Reference Example 8. Quantitative Real-Time Polymerase Chain Reaction

In the same manner as in Reference Example 4, miRNA triggers were transfected into cells. After 48 hours, total RNA was extracted using TRIzol (Thermo Fisher Scientific) according to the manufacturer's instruction. The purified RNA was treated with DNase I and then reverse transcribed in the presence of RevertAid reverse transcriptase (Thermo Fisher Scientific), with random hexamers (Thermo Fisher Scientific) serving as primers. For use in amplification of target genes, primers for target genes were added, together with the cDNA, to the SYBR Green PCR master mix (Thermo Fisher Scientific). RT-qPCR was conducted on a StepOnePlus real-time PCR system. Cq values obtained from this analysis was quantitatively analyzed based on the 2^{-ΔΔC(t)} method. The primer sequences used in this experiment are listed in Table 1, below.

TABLE 1

| Target | | | |
|---|---|---|---|
| name | Primer name | Sequence (5′→3′) | SEQ ID NO |
| GAPDH | GAPDH-Forward | CTC CTC CAC CTT TGA CGC | 1 |
| | | TG | |
| | GAPDH-Reverse | TCC TCT TGT GCT CTT GCT | 2 |
| | | GG | |
| PKR | PKR-Forward | GAG GGG AAT GAT GTG ATT | 3 |
| | | GG | |
| | PKR-Reverse | CTG GGC TGT CAC TTC TAG | 4 |
| | | CC | |
| Mcl-1 | Mcl-1-Forward | CTG TCA TTT CTT TTG GTG | 5 |
| | | CCT | |
| | Mcl-1-Reverse | ATT CCT GAT GCC ACC TTC | 6 |
| | | TA | |
| Bcl-xL | Bcl-xL- | TCC CCA TGG CAG CAG TAA | 7 |
| | Forward | AG | |
| | Bcl-xL- | TCC ACA AAA GTA TCC TGT | 8 |
| | Reverse | TCA AAG G | |
| Bcl-2 | Bcl-2-Forward | GAG AGT GCT GAA GAT TGA | 9 |
| | Bcl-2-Reverse | ATC AAT CTT CAG CAC TCT | 10 |
| | | C | |

### Reference Example 9. Assay for Cytotoxicity and Cell Viability (CCK-8 Assay)

The miRNA triggers were assayed for cytotoxicity and apoptosis induction, using the CCK-8 (Cell Counting Kit-8) (Dojindo, Kumamoto, Japan) according to the manufacturer's instruction. Cells were incubated in 96-well plates (5×10³ cells/well) at 37° C. for 24 hours under a 5% CO₂ atmosphere, followed by transfection. Three days after transfection, the medium was removed. A 10-fold dilution of CCK-8 solution in DMEM was added to the cells which were then incubated at 37° C. for 2 hours. After incubation, absorbance was read at 450 nm on Tecan Infinite M200 pro microplate reader (Mnnedorf, Switzerland).

### Example 1. Structural Optimization of miRNA Trigger

### Example 1-1. Binding of Hairpin miRNA Trigger to miRNA According to Loop Length

In this Example, an examination was made of stem lengths which allow for an optimal hairpin structure in which the hairpin miRNA triggers can bind to miRNAs to open their hairpin structures and thus can bind to target mRNAs.

Hairpin miRNA triggers (HPs) were prepared to have stems having various lengths (12 to 22 nt) as stated Reference Example 1-2 above. The hairpin miRNA triggers used in this Example were designed to bind to miR-141 and target random sequences. Their specific sequences are listed in Table 2, below. HPs and miRNAs used in this Example were not chemically modified.

TABLE 2

| Oligo name | Sequence (5′→3′) | SEQ ID NO |
|---|---|---|
| 12 nt stem_HP | TGCATCGTCCATCCATCTTTACCAGACAGTGTTAAT | 11 |
| | GGACGATGCA | |
| 14 nt stem_HP | GTTGCATCGTCCATCCATCTTTACCAGACAGTGTTA | 12 |
| | ATGGACGATGCAAC | |
| 16 nt stem_HP | TGGTTGCATCGTCCATCCATCTTTACCAGACAGTGT | 13 |
| | TAATGGACGATGCAACCA | |
| 18 nt stem_HP | CATGGTTGCATCGTCCATCCATCTTTACCAGACAGT | 14 |
| | GTTAATGGACGATGCAACCATG | |
| 20 nt stem_HP | ACCATGGTTGCATCGTCCATCCATCTTTACCAGACA | 15 |
| | GTGTTAATGGACGATGCAACCATGGT | |
| 22 nt stem_HP | TGACCATGGTTGCATCGTCCATCCATCTTTACCAGA | 16 |
| | CAGTGTTAATGGACGATGCAACCATGGTCA | |
| miR-141 | UAACACUGUCUGGUAAAGAUGG | 17 |

Each hairpin miRNA trigger includes mutually complementary sequences which are hybridized with each other, forming a stem-loop structure, with a miRNA-binding sequence located at the loop moiety. Thus, examination was made to show whether when a miRNA was added, the miRNA trigger could open its hairpin structure as it bound to the miRNA.

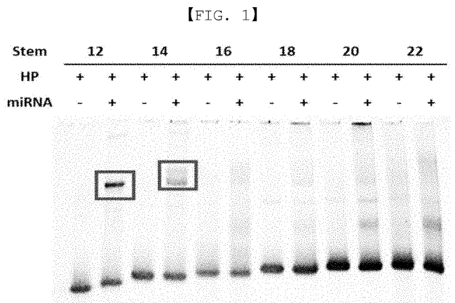

In a buffer (1×PBS, 137 mM NaCl, 10 mM PO₄, 2.7 mM KCl, 5 mM MgCl₂; pH 7.4) for low cytotoxicity and stable hairpin structure formation, the hairpin miRNA trigger and a miRNA capable of hybridizing therewith were each added at a concentration of 100 nM and incubated at 37° C. for 1 hour. The reaction mixture was loaded into pockets in a PAGE (Polyacrylamide gel electrophoresis) gel and run. The result is shown in FIG. 1.

As can be seen in FIG. 1, bands 150 bp in size were observed for miRNA triggers with a stem length of 14 nt or less in the presence of the miRNA, indicating the hybridization of HP with the miRNA. However, when the stem was 16 nt or greater in length, hybridization between HP and the miRNA was remarkably reduced. A shorter stem in HP allows a shorter sequence capable of binding to a target mRNA, leading to a reduction in target specificity. Accordingly, the stem in the hairpin miRNA trigger was set to range in length from 12 to 14 nt in order that the hairpin miRNA trigger could easily open the hairpin structure by specifically binding to both a target mRNA and the miRNA. Subsequent experiments were conducted with the hairpin miRNA trigger.

### Example 1-2. Binding of Hairpin miRNA Trigger to miRNA and Target mRNA

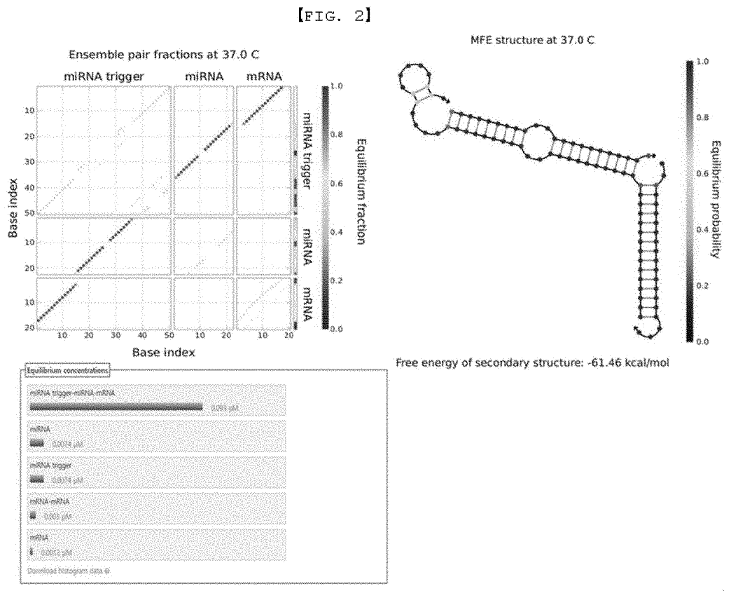

It was primarily verified through the NUPACK program in the same manner as in Reference Example 2 whether the hairpin miRNA triggers bind to miRNAs and target mRNAs to form the ternary structures of miRNA-miRNA trigger-mRNA, and the simulation results are depicted in FIG. 2. The hairpin miRNA triggers, miRNAs, and target mRNA sequences used in this Example are listed in Table 3, below. In the left panels (upper and lower) of FIG. 2, formation of base pairs among miRNA, mRNA, miRNA trigger is depicted, with strong binding (red diagonal lines) detected between miRNA and miRNA trigger (HP) and between mRNA and miRNA. Unintended non-specific binding was almost not observed at al. Although observed, the non-specific binding was present at a very low probability (blue diagonal lines). In FIG. 2, the right panel is a numerical representation of the left panel, showing overwhelmingly predominant formation of the intended ternary structure of miRNA-miRNA trigger-mRNA. In FIG. 2, the left panel shows the probability that specific bases on one strand form pairs with specific bases on a different strand while the right panel shows the prediction of the ternary structure formed when miRNA, HP, and mRNA are incubated together. In FIG. 2, “Equilibrium concentrations” represents quantities of the ternary structure formed when miRNA, HP, and mRNA were incubated together (since each of miRNA, HP, mRNA was used at a concentration of 100 nM, 97% of the constituents were present in the ternary structure form). As shown in FIG. 2, computer modeling confirmed that the hairpin miRNA trigger can bind to a miRNA and a target mRNA to form a ternary structure of miRNA-miRNA trigger-mRNA. Each of the sequences listed in Table 3 was not chemically modified.

TABLE 3

| Oligo name | Sequence (5′→3′) | SEQ ID NO |
|---|---|---|
| HP (HP-Mcl1- | CAAGAGGATTATCCATCTTTACCTCACAGTGTTA | 18 |
| 141) | ATAATCCTCTTG | |
| miRNA (miR-141) | UAACACUGUCUGGUAAAGAUGG | 17 |
| mRNA (Mcl1) | GCAAGUGGCAAGAGGAUUA | 19 |

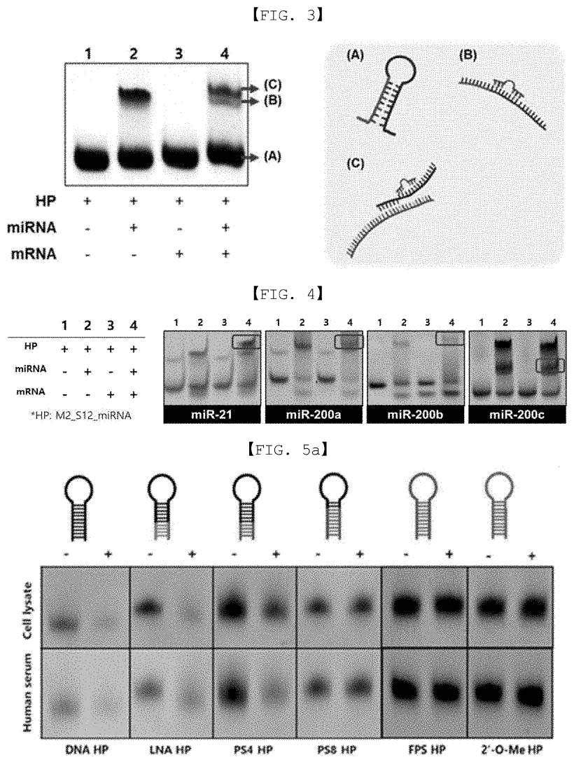

To a buffer (1×PBS+137 mM NaCl, 10 mM PO₄, 2.7 mM KCl, 5 mM MgCl₂; pH 7.4) containing the hairpin miRNA trigger (HP) verified through computer modeling, a miRNA and/or a target mRNA were each added at a concentration of 100 nM, followed by incubation at 37° C. for 1 hour. Each sample was loaded to a PAGE gel and electrophoresed. The results are shown in FIG. 3.

As can be seen in FIG. 3, the hairpin miRNA trigger according to an embodiment bind to the miRNA and/or the target mRNA in the presence of the miRNA was present (lanes 2 and 4 in the gel image) and does not bind to the target mRNA in the absence of the miRNA (lane 3 in the gel image). From this data, it is understood that the presence of a miRNA is essential for the binding of the hairpin miRNA trigger to an mRNA.

### Example 1-3. Hairpin mRNA Trigger Capable of Binding to Various miRNAs

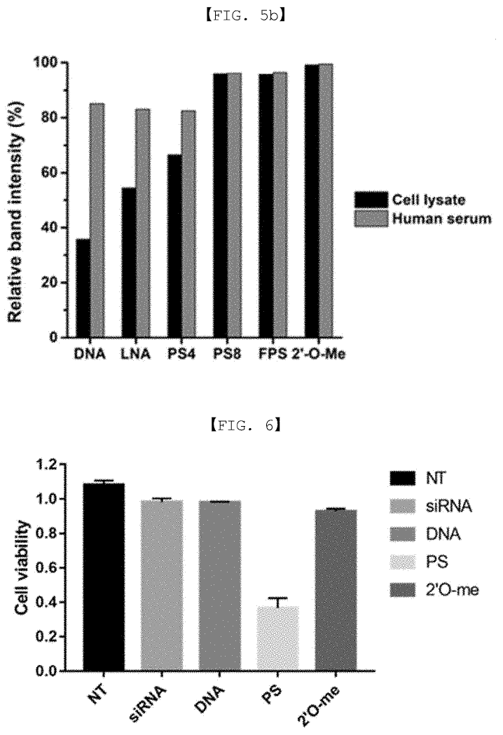

A hairpin miRNA trigger designed to bind to a miRNA-200 family (miR-200a, miR-200b, and miR-200c), or miR-21 and target Mcl-1 mRNA was prepared. To a buffer containing the hairpin miRNA trigger (HP), miRNA and/or target mRNA was added, followed by incubation at 37° C. for 1 hour. Each sample was loaded to a PAGE gel and electrophoresed. The results are given in FIG. 4. The HP used in this Example was designed to have a 12-nt long stem (S12) and to allow the portion responsible for binding to miRNA to mismatch with the miRNA at 10^{th} to 11^{th} nucleotide residues from the 3′ end (M2). The HPs, miRNAs, and target mRNAs used in this Example are listed in Table 4, below. The sequences in Table 4 were each not chemically modified.

TABLE 4

| Oligo name | Sequence (5′→3′) | SEQ ID NO |
|---|---|---|
| HP_Mcl1_M2_S12_miR- | CAAGAGGATTATTCAACATCAGATTGATAAGC | 20 |
| 21 | TATAATCCTCTTG | |
| HP_Mcl1_M2_S12_miR- | CAAGAGGATTATACATCGTTACCGTACAGTGT | 21 |
| 200a | TAATAATCCTCTTG | |
| HP_Mcl1_M2_S12_miR- | CAAGAGGATTATTCATCATTACCGAGCAGTAT | 22 |
| 200b | TAATAATCCTCTTG | |
| HP_Mcl1_M2_S12_miR- | CAAGAGGATTATTCCATCATTACCTTGCAGTA | 23 |
| 200c | TTAATAATCCTCTTG | |
| miR-21 | UAGCUUAUCAGACUGAUGUUGA | 24 |
| miR-200a | UAACACUGUCUGGUAACGAUGU | 25 |
| miR-200b | UAAUACUGCCUGGUAAUGAUGA | 26 |
| miR-200c | UAAUACUGCCGGGUAAUGAUGGA | 27 |
| Mcl-1 mRNA | ACCCUAGCAACCUAGCCAGAAAAGCAAGUGGC | 28 |
| | AAGAGGAUUAUGGCUAACAAGAAUAAAU | |

As shown in FIG. 4, the hairpin miRNA triggers capable of binding to various types of miRNAs were observed to bind to both the miRNAs and/or the target mRNA in the presence of the miRNAs (lanes 2 and 4 in the gel image), but did not bind to the target mRNA in the absence of the miRNAs even when the target mRNA was added (lane 3 in the gel image).

### Example 1-4. Stability of Trigger by Chemical Modification

In order to apply the miRNA trigger to in vitro and in vivo experiments, stability in biological samples was needed for the miRNA trigger. In this regard, HPs were prepared in the same similar manner as that of Reference Example 1, with the exception that a chemical modification, such as LNA (locked nucleic acid), PS (phosphorothioate), or 2′-O-Me (2′-O-Methyl), was introduced into nucleotides of the miRNA trigger. Sequences of HPs used in this Example and types and positions of chemical modifications in the sequences are summarized in Table 5, below.

TABLE 5

| Oligo | | Chemical | SEQ ID |
|---|---|---|---|
| name | Sequence (5′→3′) | modification | NO |
| DNA HP | TATTTCTCATTCCCCCATCTTTACCAGACAG | — | 29 |
| | TGTTAGGGAATGAGAAATA | | |
| LNA HP | [LNA T][LNA A][LNA T][LNA T] | LNA | 30 |
| | TCTCATTCCCCCATCTTTACCAGACAGTG | | |
| | TTAGGGAATGAGA[LNA A][LNA A][LNA T] | | |
| | [LNA A] | | |
| PS4 HP | T*A*T*T*TCTCATTCCCCCATCTTTACCAG | PS | 31 |
| | ACAGTGTTAGGGAATGAGA*A*A*T*A | | |
| PS8 HP | T*A*T*T*T*C*T*C*ATTCCCCCATCTTTA | PS | 32 |
| | CCAGACAGTGTTAGGGAAT*G*A*G*A*A*A | | |
| | *T*A | | |
| FPS HP | T*A*T*T*T*C*T*C*A*T*T*C*C*C*C*C | PS | 33 |
| | *A*T*C*T*T*T*A*C*C*A*G*A*C*A*G* | | |
| | T*G*T*T*A*G*G*G*A*A*T*G*A*G*A*A | | |
| | *A*T*A | | |
| 2′OMe | mUmAmUmUmUmCmUmCmAmUmUmCmCmCmCm | 2’-O-Me | 34 |
| | CmAmUmCmUmUmUmAmCmCmAmGmAmCmAmG | | |
| | mUmGmUmUmAmGmGmGmAmAmUmGmAmGmAm | | |
| | AmAmUmA | | |

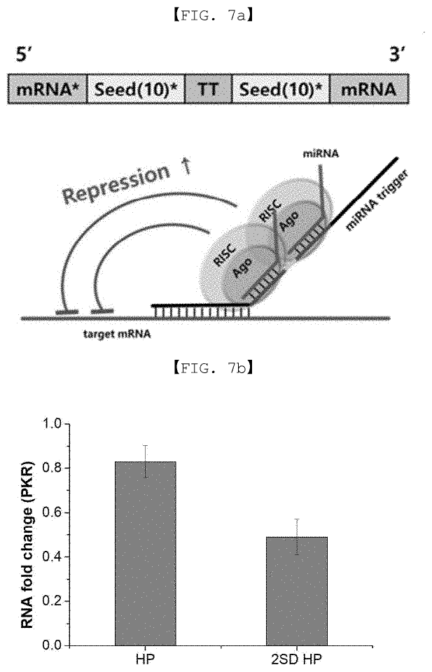

The chemical modification-introduced hairpin miRNA triggers (LNA HP, PS4HP, PS8 HP, FPS HP, and 2′-O-Me HP) or the control hairpin miRNA trigger (DNA HP), which was not chemically modified, was added to cell lysates or human sera, and analyzed for resistance to biological resistance in the same manner as in Reference Example 3. The results are given in FIGS. 5a and 5b.

As shown in FIGS. 5a and 5b, when exposed for 24 hours to the cell lysate and the human sera, DNA HP, which consists of chemically unmodified nucleotides, was degraded to 34% and 85%, respectively. The chemical modification-introduced miRNA triggers were less prone to degradation, compared to the chemically unmodified miRNA trigger. Among others, PS8 HP in which the eight nucleotides from each (stem) end of the hairpin miRNA trigger were modified with PS (PS8 HP), FPS in which the entire nucleotides of the miRNA trigger were modified with PS (FPS), and 2′-O-Me HP in which the entire nucleotides of the miRNA trigger were modified with 2′-O-Me were almost not degraded and were found to be effectively resistant to degradation in biological samples.

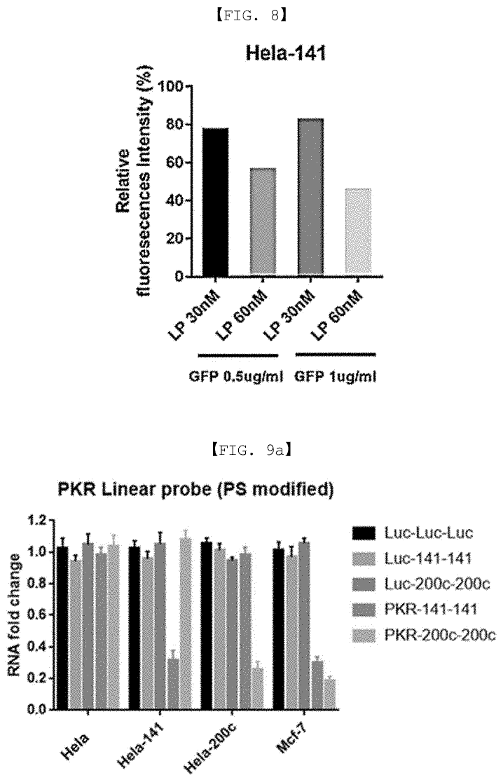

In addition, FPS HP and 2′-OMe HP, which were the most resistant to degradation in biological samples, were analyzed for cytotoxicity. In this regard, the HP in which the entire nucleotides of miRNA trigger (LP) were modified with PS (expressed as PS in Table 6 and FIG. 6), the linear probe in which the entire nucleotides of miRNA trigger (LP) were modified with 2′-O-Me (expressed as 2′O-me in Table 6 and FIG. 6), the control DNA LP (expressed as DNA in Table 6 and FIG. 6), or siRNA was transfected into cells which were then measured for cell viability using the method of Reference Example 9. The result is depicted in FIG. 6. Sequences of the HPs and siRNA used in the cell viability assay are listed, along with positions and types of chemical modification in the sequences, in Table 6, below.

TABLE 6

| Oligo | | Chemical | |
|---|---|---|---|
| name | Sequence (5′→3′) | modification | SEQ ID NO |
| siRNA | UCGAAGUACUCAGCGUAAGU | — | 35 |
| DNA | TCGAAGTACTCAGCGTAAGTTCGAAGTACT | — | 36 |
| | CAGCGTAAGTTCGAAGTACTCAGCGTAAGT | | |
| PS | T*C*G*A*A*G*T*A*C*T*C*A*G*C*G* | *: | 37 |
| | T*A*A*G*T*T*C*G*A*A*G*T*A*C*T* | phos- | |
| | C*A*G*C*G*T*A*A*G*T*T*C*G*A*A* | phorothioate | |
| | G*T*A*C*T*C*A*G*C*G*T*A*A*G*T | linkage | |
| | | (PS) | |
| 2′-O-Me | mUmCmGmAmAmGmUmAmCmUmCmAmGmCmG | m: 2′-O- | 38 |
| | mUmAmAmGmUmUmCmGmAmAmGmUmAmCmU | Methyl | |
| | mCmAmGmCmGmUmAmAmGmUmUmCmGmAmA | (2′-O-Me) | |
| | mGmUmAmCmUmCmAmGmCmGmUmAmAmGmU | | |

As shown in FIG. 6, the cells transfected with the 2′-O-Me-modified miRNA trigger was not significantly different in viability from the negative control to which no HPs had been transfected (NT; non-transfected). However, the PS miRNA trigger, although prepared with random sequences, was measured to exhibit cell viability less than 40% and thus high cytotoxicity. Collectively, the data of FIGS. 5a, 5b, and 6 indicate that the miRNA trigger including 2′-OMe-modified nucleotides is highly stable and exhibits low cytotoxicity.

### Example 2. Structure for Enhancing Efficiency of miRNA Trigger

miRNA triggers which could each bind to a plurality of miRNAs were prepared and examined for repressive effect on expression of the target genes.

A miRNA trigger was designed to have a 2-seed hairpin structure in which the loop could hybridize with two miRNAs simultaneously (2-seed hairpin probe; hereinafter referred to as “2SD HP”). The structure of 2SD HP is as follows:

[5′-portion capable of binding to mRNA of target gene (T*)-portion capable of binding to miRNA(1)(mi(1)*)-spacer(TT)-portion capable of binding to miRNA(2)(mi(2)*)-portion capable of binding to T*(T)-3′].

Including sequences (T and T* in the structure) which can bind complementarily to each other as in HP, 2SD HP exists in the stem-loop structure as a hairpin miRNA trigger. The loop of 2SD HP included a portion capable of binding to miRNA(1) (mi(1)*) and a portion capable of binding to miRNA (2) (mi (2)*). mi (1)* and mi (2)* included in 2SD HP were each composed of a sequence complementarily hybridizable with a 10-nt-long sequence from the 5′ end of miRNA. 2SD HP included a spacer (TT) between mi(1)* and mi(2)*. FIG. 7a is a schematic diagram illustrating the mechanism of repressive action of 2SD HP on the expression of a target gene.

In brief, a hairpin miRNA trigger (HP) that could bind to miR-141 and target pkr (bind to pkr mRNA to regulate the expression of pkr) and 2SD HP that could target pkr and bind to two molecules of miR-141 per target were synthesized in Bioneer Inc.

The synthesized triggers were purified through HPLC before use. Sequences of HP and 2SD HP and types of chemical modification (*; phosphorothioate linkage) are given in Table 7, below.

TABLE 7

| Oligo name | Sequence (5′→3′) | SEQ ID NO |
|---|---|---|
| HP_PKR(3)_ | TAGCTGTACTTCAACCATCTTTACCAGACAG | 39 |
| miR141 | TGTTATTGAAGTACAGCTA | |
| 2SD HP_PKR(3)_ | T*A*C*C*T*G*T*A*C*T*T*C*A*A*C*A | 40 |
| miR141 | *C*A*G*T*G*T*T*A*T*T*G*A*C*A*G* | |
| | T*G*T*T*A*T*T*G*A*A*G*T*A*C*A*G | |
| | *C*T*A | |

The synthesized HP and 2SD HP were transfected into miR-141-expressing Hela-141 cells which were then quantitatively measured for expression levels of the target pkr in the same manner as in Reference Example 8. The result is depicted in FIG. 7b. As shown in FIG. 7b, when transfected, 2SD HP that has a loop capable of binding to two miRNAs significantly decreased the expression level of the target gene, compared to HP.

From the result, it was confirmed that a portion capable of binding two miRNAs increases the repressive effect on the expression of a target gene, compared to a portion capable of binding one miRNA. This principle was introduced into linear miRNA triggers. That is, linear miRNA triggers were designed to each include two portions capable of binding to respective miRNAs, and used in subsequent experiments.

### Example 3. A In Vitro Assay for Efficacy of miRNA Trigger

### Example 3-1. Assay for Expression Regulation of Fluorescent Protein by miMNA Trigger

A GFP fluorescent protein-expressing plasmid (used in Chen L L, DeCerbo J N, Carmichael G G. 2008. Alu element-mediated gene silencing. EMBO J 27: 1694-1705) was transfected into Hela-141 cells to prepare GFP-expressing Hela-141 cells. As stated in Reference Example 1, a linear miRNA trigger (LP; GFP_141_141), which can target GFP mRNA (=complementarily bind to GFP mRNA to regulate the expression of GFP) and complementarily bind to miR-141, and siRNAs which target GFP and Luc (Luciferase), respectively, were synthesized in Bioneer Inc. Sequences of the LP and siRNAs and types and positions of chemical modifications in each sequence are listed in Table 8, below.

TABLE 8

| | | modifi- | SEQ ID |
|---|---|---|---|
| Oligo name | Sequence (5′→3′) | cation | NO |
| GFP(1)_141_ | mCmCmAmUmCmUmUmUmAmCmCmUmCmAm | m; 2′- | 41 |
| 141 | CmAmGmUmGmUmUmAmCmCmAmUmCmUmU | OMe | |
| | mUmAmCmCmUmCmAmCmAmGmUmGmUmUm | | |
| | AmUmUmAmUmGmUmUmUmCmAmGmGmUmU | | |
| | mCmAmGmGmGmGmGmA | | |
| GFP(2)_141_ | mCmCmAmUmCmUmUmUmAmCmCmUmCmAm | m; 2′- | 42 |
| 141 | CmAmGmUmGmUmUmAmCmCmAmUmCmUmU | OMe | |
| | mUmAmCmCmUmCmAmCmAmGmUmGmUmUm | | |
| | AmAmAmAmUmUmUmGmUmGmAmUnGmCmU | | |
| | mAmUmUmGmCmU | | |
| siLuc | UCGAAGUACUCAGCGUAAG | None | 43 |
| siGFP | UUAUGUUUCAGGUUCAGGG | None | 44 |

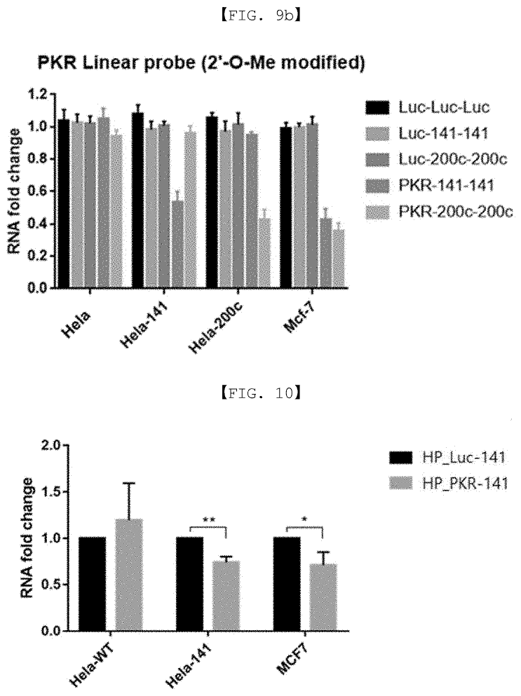

Above all, siLuc and siGFP were transfected into the GFP-expressing Hela-141 cells which were then measured for fluorescence intensity on an ELISA reader. A significantly reduced fluorescence intensity was detected from the siGFP-treated group, compared to the siLuc-treated group, confirming that the experimental condition of the Example worked normally. In addition, the linear miRNA trigger (GFP(1)_141_141) was transfected into the GFP-expressing Hela-141 cells which were then measured for fluorescence intensity. The result is depicted in FIG. 8. For the transfection, the GFP plasmid and GFP_141_141 were used at a concentration of 0.5 to 1 μg/ml and 30 to 60 nM, respectively. As shown in FIG. 8, the linear miRNA trigger according to an embodiment reduced GFP fluorescence intensity in a dose-dependent manner.

### Example 3-2. Assay for Ability of Linear miRNA Trigger to Discriminate Cell Lines

A linear miRNA trigger (LP; PKR-141-141, PKR-200c-200c) that targets PKR and can bind to miR-141 or miR-200c was prepared in the same manner as in Reference Example 1, together with the controls including a linear miRNA trigger (LP; Luc-141-141, PKR-200c-200c) that targets Luc and can bind to miR-141 or miR-200c and a probe (Luc-Luc-Luc) that includes three portions capable of binding to Luc. Sequences of the prepared miRNA triggers and probe and positions and types of chemical modification (in; 2′-O-Me) are summarized in Table 9.

TABLE 9

| Oligo name | Sequence (5′-3′) | SEQ ID NO |
|---|---|---|
| PKR(1)_141_141 | mCmCmAmUmCmUmUmUmAmCmCmUmCmAmCmAmG | 45 |
| | mUmGmUmUmAmCmCmAmUmCmUmUmUmAmCmCmU | |
| | mCmAmCmAmGmUmGmUmUmAmUmAmUmGmUmGmA | |
| | mGmGmCmAmGmAmGmAmAmCmGmA | |
| PKR(2)141_141 | mCmCmAmUmCmUmUmUmAmCmCmUmCmAmCmAmG | 46 |
| | mUmGmUmUmAmCmCmAmUmCmUmUmUmAmCmCmU | |
| | mCmAmCmAmGmUmGmUmUmAmUmAmUmUmUmCmU | |
| | mCmAmUmUmCmCmCmUmUmCmCmU | |
| PKR(1)_200c_ | mUmCmCmAmUmCmAmUmUmAmCmCmGmCmGmCmA | 47 |
| 200c | mGmUmAmUmUmAmUmCmCmAmUmCmAmUmUmAmC | |
| | mCmGmCmGmCmAmGmUmAmUmUmAmUmAmUmGmU | |
| | mGmAmGmGmCmAmGmAmGmAmAmCmGmA | |
| PKR(2)_200c_ | mUmCmCmAmUmCmAmUmUmAmCmCmGmCmGmCmA | 48 |
| 200c | mGmUmAmUmUmAmUmCmCmAmUmCmAmUmUmAmC | |
| | mCmGmCmGmCmAmGmUmAmUmUmAmUmAmUmUmU | |
| | mCmUmCmAmUmUmCmCmCmUmUmCmCmU | |
| Luc_Luc_Luc | mUmCmGmAmAmGmUmAmCmUmCmAmGmCmGmUmA | 49 |
| | mAmGmUmCmGmAmAmGmUmAmCmUmCmAmGmCmG | |
| | mUmAmAmGmUmCmGmAmAmGmUmAmCmUmCmAmG | |
| | mCmGmUmAmAmG | |
| Luc_141_141 | mCmCmAmUmCmUmUmUmAmCmCmUmCmAmCmAmG | 50 |
| | mUmGmUmUmAmCmCmAmUmCmUmUmUmAmCmCmU | |
| | mCmAmCmAmGmUmGmUmUmAmUmCmGmAmAmGmU | |
| | mAmCmUmCmAmGmCmGmUmAmAmG | |
| Luc_200c_200c | mUmCmCmAmUmCmAmUmUmAmCmCmGmCmGmCmA | 51 |
| | mGmUmAmUmUmAmUmCmCmAmUmCmAmUmUmAmC | |
| | mCmGmCmGmCmAmGmUmAmUmUmAmUmCmGmAmA | |
| | mGmUmAmCmUmCmAmGmCmGmUmAmAmG | |

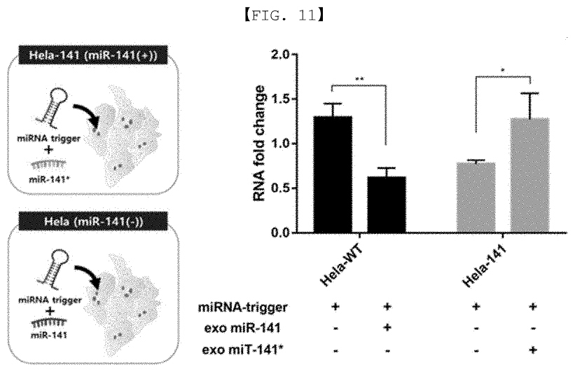

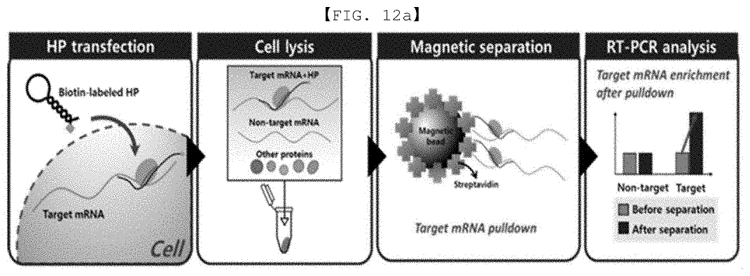

Linear miRNA triggers (Table 9, PKR(1)_141_141, PKR(1)_200c_200c and control) which had the same sequences as in the above linear miRNA triggers and additionally included PS (phosphorothioate) bonds between all nucleotides were prepared and transfected into Hela, Hela-141, Hela-200c, and MCF-7 cell lines. Expression levels of pkr mRNA was measured by real-time PCR and are depicted in FIG. 9a.

In addition, the linear miRNA triggers and probe (Table 9, PKR(1)_141_141, PKR(1)_200c_200c, and control) chemically modified with 2′-OMe were prepared and transfected into Hela, Hela-141, Hela-200c, and MCF-7 cell lines. Expression levels of pkr mRNA was measured by real-time PCR and are depicted in FIG. 9b.

As shown in FIGS. 9a and 9b, the miRNA triggers modified with PS or chemically modified with 2′-OMe exhibited remarkably excellent ability to discriminate cell lines: (1) in Hela-141 cells, the expression of PKR mRNA was about 50-70% reduced by the PKR-141-141 miRNA trigger, but was not changed by other miRNA triggers; (2) in Hela-200c cells, the expression of PKR mRNA was about 60-80% reduced by the PKR-200c-200c miRNA trigger, but other miRNA triggers; and (3) in Mcf-7 cells, the expression of PKR mRNA was about 60-70% and about 70-85% reduced by PKR-141-141 and PKR-200c-200c miRNA triggers, respectively.

The transfection of the PS-modified miRNA trigger into cells was observed to induce cell death by about 7-% due to the cytotoxicity thereof irrespective of the sequences of the miRNA trigger, as shown in FIG. 6. However, it was found that the cells having the 2′-OMe-modified miRNA triggers infected thereinto did not undergo death. Thus, the 2′-OMe-modified miRNA trigger was identified to exhibit excellent cell strain discriminating performance, excellent cell stability, and low cytotoxicity.

### Example 3-3. Assay for Ability of Hairpin miRNA Trigger to Discriminate Cell Lines

A hairpin miRNA trigger (HP_PKR(3)_141) that targets PKR and can bind to miR-141 was prepared in the same manner as in Reference Example 1-2. The sequence of the HP used in this Example and the position of chemical modification on the sequence are listed in Table 10. All the nucleotides of the sequence were chemically modified with 2′-OMe.

TABLE 10

| Oligo name | Sequence (5′-3′) | SEQ ID NO |
|---|---|---|
| HP_PKR(3)_141 | mUmUmGmAmAmGmUmAmCmAmGmCrUmAmCmCmAmU | |
| | mCmUmUmUmAmCmCmUmCmAmCmAmGmUmGmUmUmA | |
| | mUmAmGmCmUmGmUmAmCmUmUmCmAmA | |

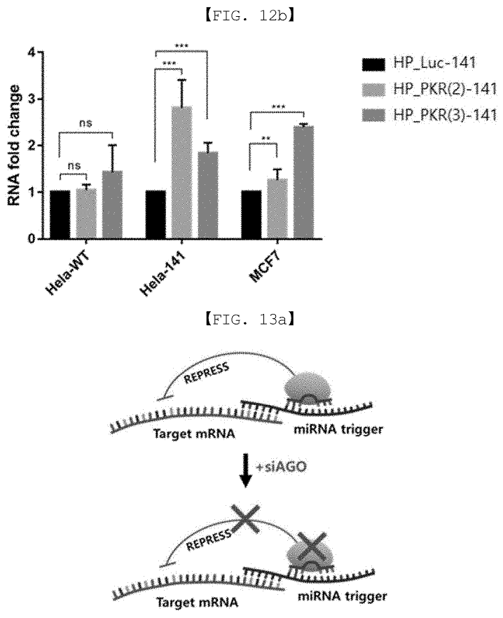

The hairpin miRNA triggers synthesized above were transfected into Hela, Hela-141, and MCF-7 cells, followed by performing real-time PCR to measure expression levels of pkr mRNA. The results are depicted in FIG. 10. As can be seen in FIG. 10, the hairpin miRNA trigger decreased the expression of pkr mRNA only in Hela-141 and MCF-7 cells expressing miR-141.

Through this result, it is understood that the hairpin miRNA trigger can discriminates cell lines according to types of miRNAs expressed and can sufficiently regulate the expression of target genes at the levels of the miRNA expressed endogenously in cells themselves as well as the miRNA artificially expressed.

### Example 4. Mechanism of Action of miRNA Trigger

### Example 4-1. Assay for Regulation of Target Gene Expression According to MiRNA

In this Example, examination was made to show whether the downregulation of mRNA expression of target genes by miRNA triggers was dependent on miRNA.

The hairpin miRNA trigger (HP_PKR(3)_141; Table 10) that can bind to miR-141 and target the pkr gene was prepared in a similar manner to those in Reference Example 1. miR-141, and miR-141*, which complementarily binds to miR-141 to inhibit the binding between the miRNA and the miRNA trigger in a competitive manner, were synthesized in Bioneer Inc. The sequence of the hairpin miRNA trigger (HP_PKR(3)_141) used in this Example is listed in Table 10 and the sequences of miR-141 and miR-141* are given in Table 11, below. Each of the sequences listed in Table 11 were not chemically modified.

TABLE 11

| Oligo name | Sequence (5′-3′) | SEQ ID NO |
|---|---|---|
| miR-141 | UAACACUGUCUGGUAAAGAUGG | 17 |
| miR-141* | CCAUCUUUACCAGACAGUGUUA | 53 |

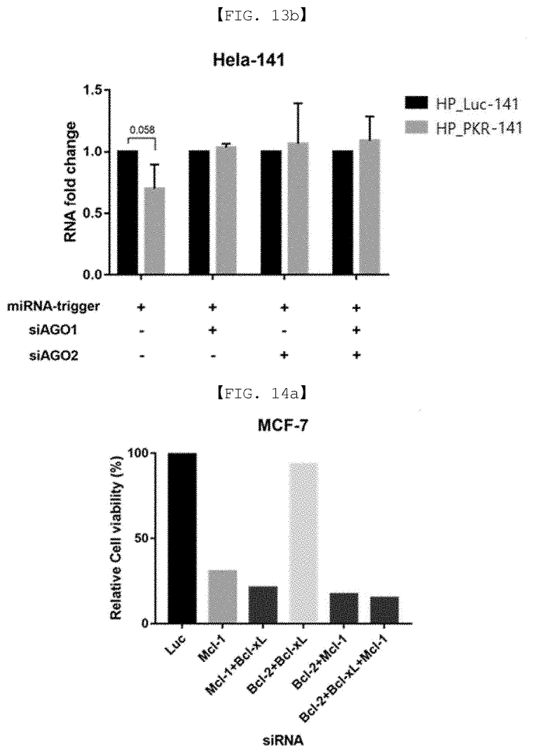

(1) Hela-141 cells were transfected with (i) the hairpin miRNA trigger alone or (ii) the miRNA trigger and miR-141* in combination, and (2) Hela cells were transfected with (i) the miRNA trigger alone or (ii) the miRNA trigger and miR-141 hybridizable therewith in combination. In each case, pkr mRNA expression levels were measured and are depicted in FIG. 11.

As shown in FIG. 11, transfection of the hairpin miRNA trigger in combination with miR-141* into Hela-141 cells, which express miR-141, remarkably increased the expression of pkr, compared to transfection of the hairpin miRNA trigger alone. This result indicates that miR-141* hybridizes with intracellular miR-141 to inhibit the opening of the hairpin structure in the hairpin miRNA trigger, with the consequent inhibition of the target expression repression effect.

In addition, as can be seen in FIG. 11, when the hairpin miRNA trigger was transfected into Hela cells to which miR-141 is not endogenous, the addition of miR-141 resulted in a remarkable decrease in pkr expression, implying that the hairpin miRNA trigger binds to (hybridizes with) the externally added, synthetic miR-141 to induce structural opening, thereby downregulating the target expression.

From the data, it was understood that the hairpin miRNA trigger acts as an expression inhibitor in the presence of miRNA.

### Example 4-2. Assay for Binding of miRNA Trigger to Target Gene

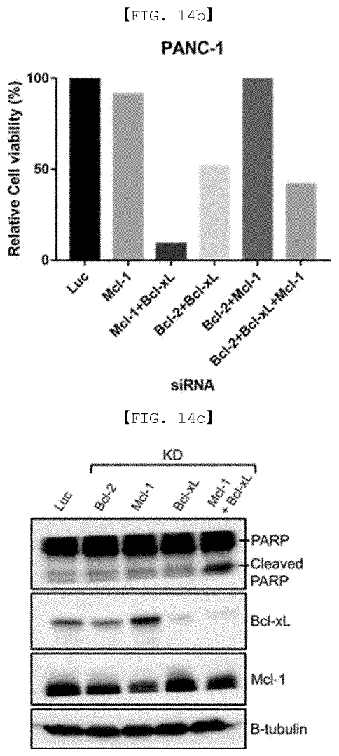

In order to prove that the miRNA trigger actually binds to the mRNA of target gene, an RNA pulldown assay using streptavidin-coated magnetic beads was designed in this Example. A schematic diagram for the RNA pulldown assay is depicted in FIG. 12a.

A hairpin miRNA trigger (HP_PKR1141) that can bind to miR-141 and target the pkr gene and a control miRNA trigger (HP_luc-141) that can bind to miR-141 and target Luc were designed, and then labeled with biotin at the 3′ end of each trigger in Bioneer Inc. The biotin-labeled hairpin miRNA triggers were transfected into cells. After 48 hours, the cells were lysed in a lysis buffer (LB-A; 100 mM Tris-HCl, pH 7.5, 500 mM LiCl, 10 mM EDTA, 1% Triton X-100, 5 mM DTT, 20 U/ml DNase I, 100 U/ml RNasin, complete EDTA-free protease-inhibitor cocktail). The cell lysate was sonicated for 20 sec three times using a sonicator, with a halt for 30 sec between the sonications.

Then, the cell lysate was transferred into a 2-ml tube and centrifuged at 15,000 g for 10 min, 4° C. The supernatant thus formed was harvested, transferred to a new tube, and used for extracting RNA therefrom. Sequences of the trigger and control used in this Example are listed in Table 12, below, and every nucleotide in the miRNA trigger and control was bonded to adjacent nucleotide via a PS (phosphorothioate) linkage (expressed as * in Table 12, below).

TABLE 12

| Oligo name | Sequence (5′→3′) | SEQ ID NO |
|---|---|---|
| HP_PKR(2)_141 | T*A*T*T*T*C*T*C*A*T*T*C*C*C*C*C*A*T*C | 54 |
| | *T*T*T*A*C*C*A*G*A*C*A*G*T*G*T*T*A*G* | |
| | G*G*A*A*T*G*A*G*A*A*A*T*A-Biotin | |
| HP_PKR(3)_141 | T*A*G*C*T*G*T*A*C*T*T*C*A*A*C*C*A*T*C | 55 |
| | *T*T*T*A*C*C*A*G*A*C*A*G*T*G*T*T*A*T* | |
| | T*G*A*A*G*T*A*C*A*G*C*T*A-Biotin | |
| HP_Luc-141 | T*C*G*A*A*G*T*A*C*T*C*A*G*C*C*C*A*T*C | 56 |
| | *T*T*T*A*C*C*A*C*A*C*A*G*T*G*T*T*A*G* | |
| | C*T*G*A*G*T*A*C*T*T*C*G*A-Biotin | |

Prior to the experiment, streptavidin-coated magnetic beads were washed three times with 750 μl of a B&W buffer (10 mM Tris-HCl, pH 7.5, 150 mM NaCl, 0.5 mM EDTA, pH 8.0) and then resuspended in 300 μl of a B&W buffer. Subsequently, the beads were incubated at room temperature for 1 hour in 1 μl of a B&W buffer containing 0.1 mg mL-1 Escherichia coli transfer RNA (tRNA). In order to binding the beads to the target RNA, 30 μl of the streptavidin-coated magnetic beads were incubated with the cell lysate at 25° C. for 3 hours while stirring at 950 rpm. The beads were washed three times at 55° C. with 750 μl of a B&W buffer to remove non-specifically bound RNAs. Thereafter, the beads were mixed at 950 rpm for 10 min with an elution buffer (10 mM Tris-HCl, pH 7.5) while being heated to 90° C. to rapidly separate the target RNA bound to the miRNA trigger complex. The isolated RNA was amplified by RT-PCR. PKR mRNA levels were measured and are depicted relative to those before the separation using magnetic beads in FIG. 12b.

As shown in FIG. 12b, Hela-141 and MCF-7 cells, which express miR-141 therein, were observed to remarkably increase in the level of the miRNA trigger-bound PKR mRNA whereas Hela cells to which miR-141 is not endogenous did not increase in the level of pkr mRNA even after the pull-down enrichment.

From the result, it is understood that the miRNA trigger can bind to the mRNA of the target gene only in the presence of the miRNA hybridizable with the miRNA trigger in the cells.

### Example 4-3. Functional Association of miRNA Trigger with Ago Protein

In this Example, an examination was made to show whether Ago (Argonaute), which is a protein mediating the miRNA function, is associated with the function of miRNA trigger. In this regard, siAGO, which can repress AGO expression, was transfected, together with the miRNA trigger, into cells to examine whether the siAGO inhibit the action of the miRNA trigger.

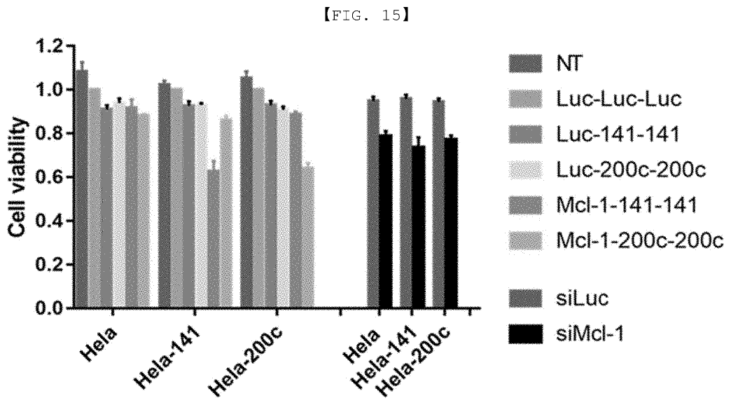

Hela-141 cells were transfected with the hairpin miRNA trigger (HP_PKR(3)_141), together with siAGO1, siAGO2 (siAGO2-1 to siAGO2-4 mixed at the same ratio (1:1:1:1)), or a combination thereof, and measured for mRNA levels of target pkr. The result is depicted in FIG. 13b. As a control, a Luc (luciferase) gene-targeting siRNA (siLuc) was transfected, together with HP_PKR(3)_141, into cells. Sequences are listed in Table 12 for HP_PKR(3)_141 and in Table 13 for siAGO1 and siAGO2.

TABLE 13

| Oligo name | Sequence (5′→3′) | SEQ ID NO |
|---|---|---|
| siLuc(MM) | UCGAAGUACUCAGCGUAAG | 43 |
| siAGOl | GGAGUUACUUUCAUAGCAU | 57 |
| SiAGO2-1 | GCACGGAAGUCCAUCUGAA | 58 |
| siAGO2-2 | GCAGGACAAAGAUGUAUUA | 59 |
| siAGO2-3 | GGGUCUGUGGUGAUAAAUA | 60 |
| siAGO2-4 | GUAUGAGAACCCAAUGUCA | 61 |

As shown in FIG. 13b, the negative control (MM) in which siLuc was added, without using siRNA capable of repressing AGO expression decreased in pkr expression level by about 30% through the action of the miRNA trigger. In contrast, co-transfection of the miRNA trigger and siAGO1, siAGO2, or a combination thereof remarkably decreased the repressive effect on PKR expression, compared to the negative control (MM) (that is, PKR expression was increased, compared to the negative control). These data indicate that the repressive action of the miRNA trigger on target mRNAs is associated with the proteins AGO1 and AGO2, which mediate the miRNA function.

### Example 5. Assay for Therapeutic Effect of miRNA Trigger on Cancer

### Example 5-1. Selection of Target Gene for Apoptosis Induction

For use in inducing cancer cells to undergo apoptosis, Bcl-2 family genes (bcl-2, bcl-xL, mcl-1), which are anti-apoptotic genes, were selected as target genes of the miRNA triggers.

To investigate the influence of Bcl-2 family gene regulation on apoptosis, siRNAs capable of regulating expression of the Bcl-2 family genes (bcl-2, bcl-xL, mcl-1) were prepared. Sequences of the siRNAs are given in Table 14, below.

TABLE 14

| | | |
|---|---|---|
| Oligo name | Sequence (5′->3′) | SEQ ID NO |

| | | |
|---|---|---|
| siBcl-2 | AUCAAUCUUCAGCACUCUC | 62 |
| siBcl-xL | UUGGUCCCUCAGUAUGGUC | 63 |
| siMcl-1 | AAAUUCGAUACUUCCUUCG | 64 |
| s iLuc | UCGAAGUACUCAGCGUAAG | 43 |

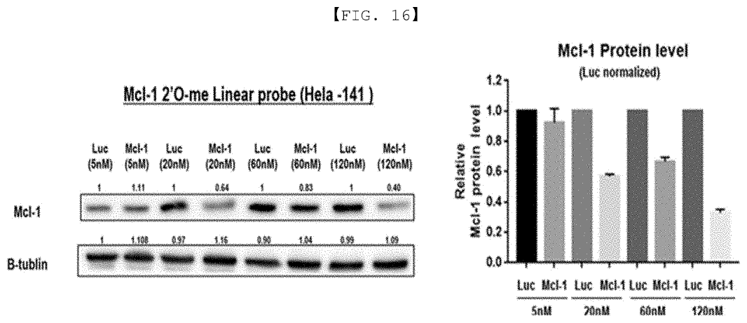

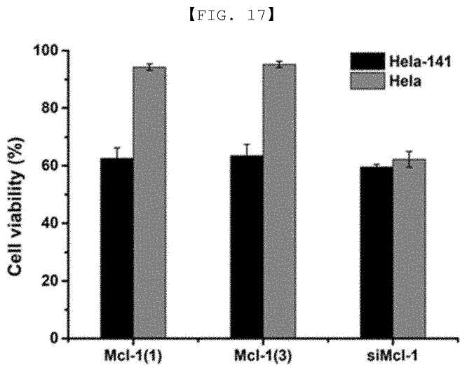

An siRNA targeting bcl-2, bcl-xL, or mcl-1 was transfected into MCF-7 breast cancer cells and PANC-1 pancreatic cancer cells. By using qRT-PCR, establishment was made of a condition under which the expression of each target gene is decreased by 80% or more (transfection was performed at a concentration of 60 nM with the aid of Lipofectamine). As a negative control, a Luc (Luciferase)-targeting siRNA (siLuc) was used. Under the same condition of decreasing the expression of each target gene by 80% or more, the cells transfected with the siRNAs were measured for apoptosis, and the results are depicted in FIGS. 14a and 14b.

As shown in FIG. 14a, MCF-7 breast cancer cells decreased in cell viability upon repression of mcl-1 gene expression and further underwent apoptosis when the expression of bcl-2 gene and/or bcl-xL gene was additionally repressed. As shown in FIG. 14b, the pancreatic cell line PANC1 greatly decreased in cell viability when expression of both mcl-1 and bcl-xL genes was downregulated.

Additionally, repression of both bcl-xL and mcl-1 in PANC1 cells was observed to increase the expression of the apoptotic marker cleaved-PARP1, as assayed by western blotting, and the result is given in FIG. 14c.

### Example 5-2. Apoptosis Induction by Linear miRNA Trigger

Based on the result of Example 5-1, linear miRNA triggers that could bind to the miR-200 family (miR-141, miR-200c) and target Mcl-1 gene (hereinafter Mcl-1-141-141, Mcl-1-200c-200c) were prepared in a similar manner to that of Reference Example 1-1 while linear miRNA triggers that could bind to the miR-200 family (miR-141, miR-200c) and target Luc (luciferase) gene (hereinafter Luc-Luc-Luc, Luc-141-141, Luc-200c-200c) were prepared as controls.

Sequences are listed in Table 15 for the linear miRNA triggers used, in Table 9 for the controls, and in Table 14 for siLuc and siMcl-1.

TABLE 15

| | | SEQ |
|---|---|---|
| Oligo | | ID |
| name | Sequence (5′->3′) | NO |
| Mcl-1_ | mCmCmAmUmCmUmUmUmAmCmCmUmCmAmCm | 65 |
| 141_ | AmGmUmGmUmUmAmCmCmAmUmCmUmUmUmA | |
| 141 | mCmCmUmCmAmCmAmGmUmGmUmUmAmCmCm | |
| | GmAmAmCmUmAmCmGmUmAmGmC | |
| Mcl-1_ | UmCmCmAmUmCmAmUmUmAmCmCmGmCmGmC | 66 |
| 200c_ | mAmGmUmAmUmUmAmUmCmCmAmUmCmAmUm | |
| 200c | UmAmCmCmGmCmGmCmAmGmUmAmUmUmAmC | |
| | mCmGmAmAmCmUmAmCmGmUmAmGmCm | |

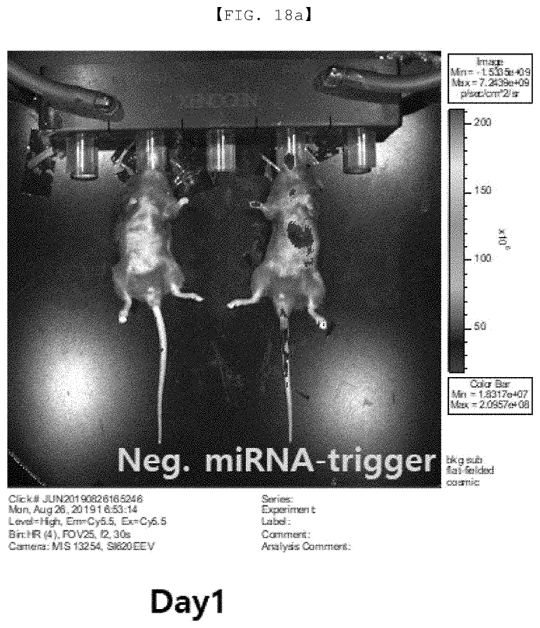

The linear miRNA triggers or siRNAs (siLuc and siMcl-1) prepared above were transfected into Hela, Hela-141, and Hela-200c cells which were then measured for cell viability. The results are depicted in FIG. 15.

As shown in FIG. 15, the siRNA (siMcl-1) induced apoptosis in all of Hela, Hela-141, and Hela-200c cells and thus was unable to discriminate cell lines. This is attributed to the siRNA mechanism that regulates Mcl-1 gene irrespective of expression of miRNA or types of the miRNAs expressed. Mcl-1-141-141 induced apoptosis of Hela-141 cells by up to about 40%, which is similar apoptotic level to that of siRNA, and had no significant influence on cell viability in Hela-200c cells. In addition, Mcl-1-200c-200c induced apoptosis of Hela-200c cells by up to about 40%, which is similar apoptotic level to that of siRNA, and had no significant influence on cell viability in Hela-141 cells. From the data, it was confirmed that the miRNA triggers according to an embodiment can discriminate cell lines according to types of the miRNAs hybridizable therewith.

### Example 5-3. Assay for Repression of Linear miRNA Trigger Against Target Protein Expression

A linear miRNA trigger (Mcl-1-141-141; Table 15) that could bind to miR-141 and target mcl-1 was prepared while a linear miRNA trigger (Luc-141-141; Table 9) that could bind to miR-141 and target Luc (luciferase) was used as a control. The triggers were transfected at various concentrations (5 to 120 nM) into Hela-141 cells. Then, proteins were extracted from the Hela-141 cells and quantitatively analyzed for Mcl-1 protein level by western blotting. The results are depicted in FIG. 16.

As shown in FIG. 16, the group transfected with Mcl-1-141-141 regulating the mcl-1 gene significantly decreased in Mcl-1 protein level, compared to the control Luc-141-141. From this data, it was confirmed that the linear miRNA triggers according to an embodiment downregulate protein expressions of the target genes.

### Example 5-4. Apoptosis Induction by Hairpin miRNA Trigger

Hairpin miRNA triggers (Mcl-1(1)-141, Mcl-1(3)-141) which could bind to miR-141 and target mcl-1 were prepared and assayed for ability to induce apoptosis. Sequences of the hairpin miRNA triggers used in this Example are listed in Table 16, with every nucleotide modified with 2′-OMe while the sequence of the control siMcl-1 is given in Table 14.

TABLE 16

| | | SEQ |
|---|---|---|
| Oligo | | ID |
| name | Sequence (5′->3′) | NO |
| HP_Mcl- | mGmCmUmAmCmGmUmAmGmUmUmCmGmGmC | 67 |
| 1(1)_ | mCmAmUmCmUmUmUmAmCmCmAmGmAmCmAm | |
| 141 | GmUmGmUmUmAmCmCmGmAmAmCmUmAmCmG | |
| | mUmAmGmC | |
| HP_Mc1- | mCmGmAmAmGmGmAmAmGmUmAmUmCmGmC | 68 |
| 1(3)_ | mCmAmUmCmUmUmUmAmCmCmAmGmAmCmAm | |
| 141 | GmUmGmUmUmAmCmGmAmUmAmCmUmUmCmC | |
| | mUmUmCmG | |

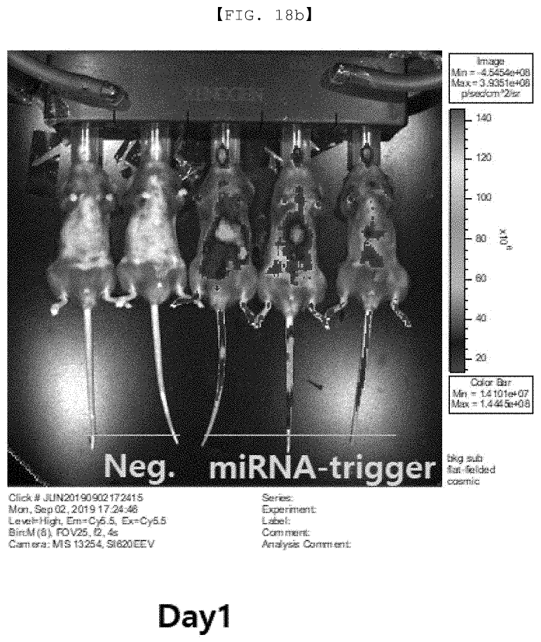

The hairpin miRNA triggers prepared above were transfected into Hela and Hela-141 cells which were then assayed for apoptosis. The results are given in FIG. 17. As a control, the siRNA (siMcl-1; Table 14) which can repress the expression of mcl-1 was transfected to Hela and Hela-141 cells and measured for apoptotic activity.

As can be seen in FIG. 17, transfection of siMcl-1 brought about an apoptotic effect in both Hela and Hela-141 cells whereas transfection of the hairpin miRNA triggers induced apoptosis of the miR-141-expressing Hela-141 cells by about 40%, but did not lead Hela cells to death at all. This result was true of both the groups transfected with HP_Mcl-1(1)_141 and HP_Mcl-1(3)_141 which are different from each other in the site of complementary hybridization with the target gene mcl-1. From the data, it was confirmed that the hairpin miRNA trigger according to an embodiment could exhibit an apoptotic effect in a specific manner for miRNA (miR-141)-expressing cells.

### Example 6. Assay for Possibility of In Vivo Delivery of mRNA Trigger

### Example 6-1. Construction of Fluorephore-Labeled miRNA Trigger and Cell Transformation

A linear miRNA trigger (5′-Cy5.5-PKR-miR-155-miR-155-3′) which could bind to miR-155 and target PKR and was labeled with Cy5.5 fluorophore was prepared as in Reference Example 1. The sequence of the trigger is given in Table 17, below.

TABLE 17

| | | SEQ |
|---|---|---|
| Oligo | | ID |
| name | Sequence (5′->3′) | NO |
| Cy5.5_ | Cy5.5- | 69 |
| PKR- | mUmUmAmUmGmUmUmUmCmAmGmUmUmUmCm | |
| 155_155 | AmGmGmGmGmGmAmUmUmAmUmGmUmUmUmC | |
| | mAmGmGmUmUmCmAmGmGmGmGmGmAmUmAm | |
| | UmGmUmGmAmGmGmCmAmGmAmGmAmAmCmG | |
| | mA | |

With the aid of Lipofectamine, the linear miRNA trigger (5′-Cy5.5-miR-155-miR-155-PKR-3′) was transfected into the FLT3-ITD mutant AML cell lines MOLM-14 and MV4-11 and the solid cancer cell line Hela. Fluorescence was detected from all of NOML-14, MV4-11, and Hela cells transfected with the linear miRNA trigger as measured by IVIS 100 (PerkinElmer). Subsequent animal tests were performed with the trigger.

### Example 6-2. In Vivo Delivery of miRNA Trigger

The linear miRNA trigger (5′-Cy5.5-PKRmiR-155-miR-155-PKR-3′) prepared in Example 6-1 was mixed in an amount of 2001 with Invivofectamine (Thermo Fisher Scientific) to form a final concentration of 120 nM and administered into 8-week-old C57BL/6 (B6) mice by tail vein injection. As a negative control, PBS was injected at a dose of 200 μl into mice.

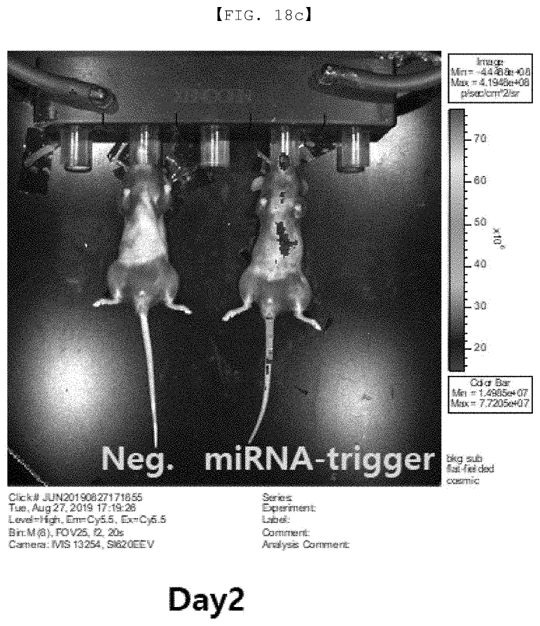

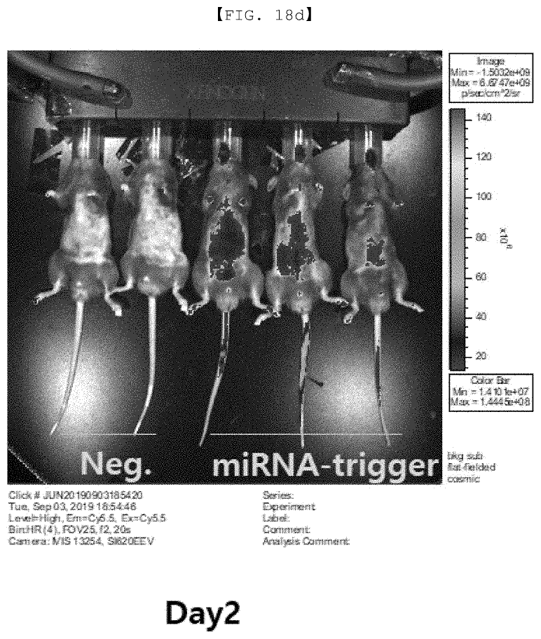

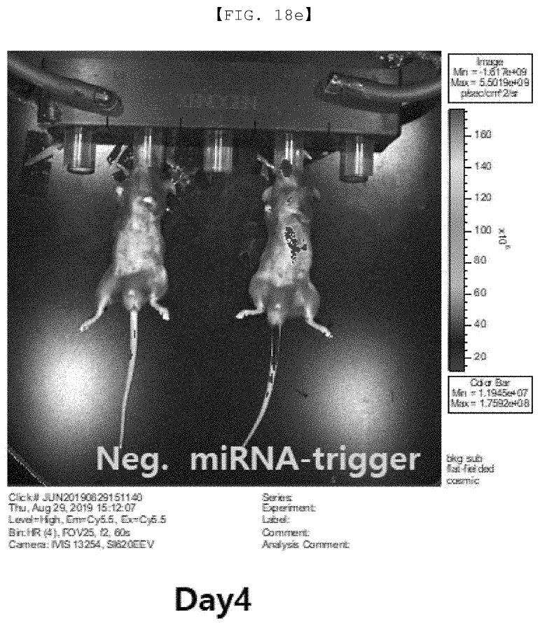

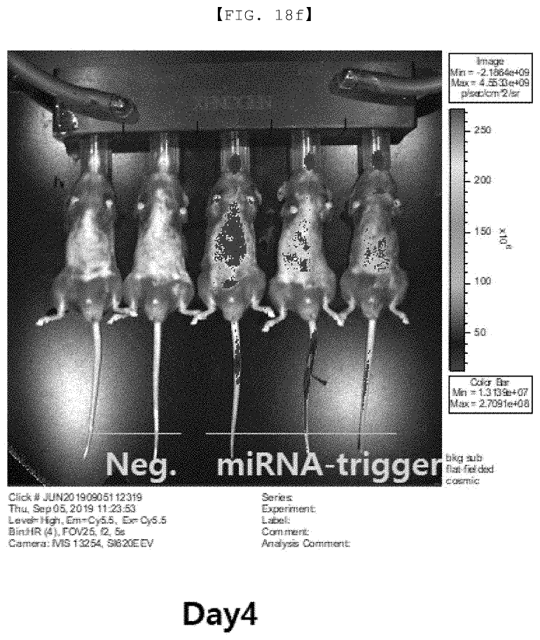

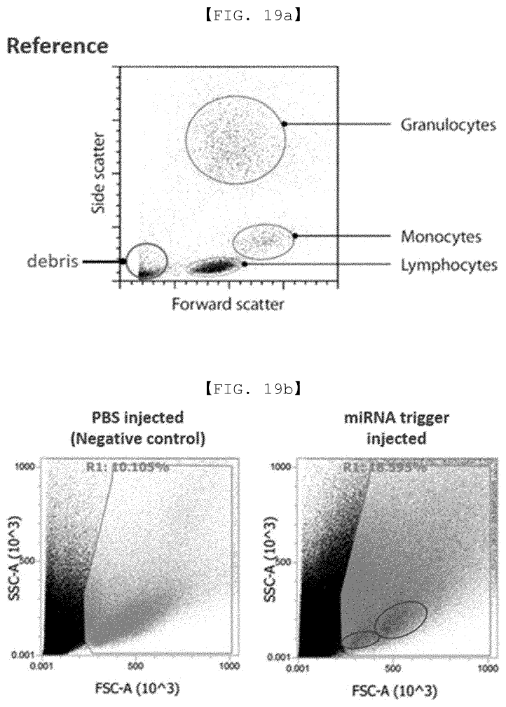

Days 1, 2, and 4 after miRNA trigger injection, in vivo imaging was performed using IVIS 100 (PerkinElmer). The results are depicted in FIGS. 18a to 18f. As shown in FIGS. 18a to 18f, the Cy5.5. conjugated to the miRNA trigger was detected in main organs including the liver, the spleen, the bone marrow, and the heart, and the hematopoietic organ of the mice. The Cy5.5 fluorescence became weak in intensity with time, but detected in the mouse body until 4 days or later. Over the period of in vivo imaging, particular toxicity was detected from none of the mice.

### Example 6-3. Intracellular Delivery of miRNA Trigger Among Hematopoietic Origin Cells

Day 4 after miRNA trigger injection in Example 6-2, mice in the miRNA trigger-administered test group (N=4) and the PBS-administered control (N=4) were sacrificed and specimens (blood cells) were taken from peripheral blood vessels, the spleen, and the bone marrow (femur blood cells). Erythrocytes were removed using an RBC lysis buffer.

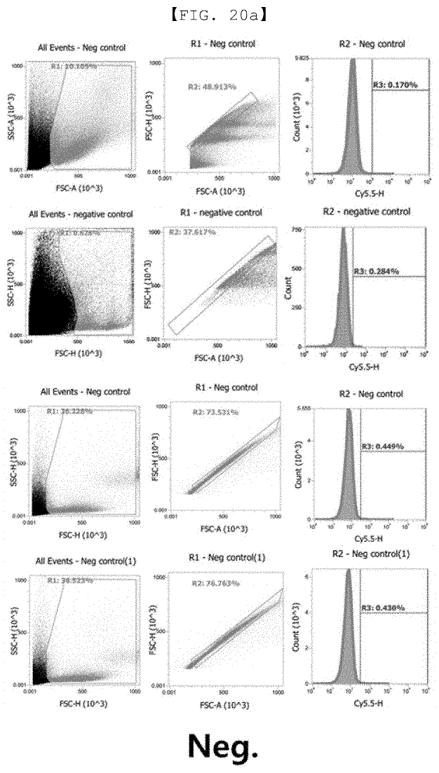

FACS (FSC-A/SSC-A) profiles of the cells are given in FIGS. 19a and 19b. As shown in FIGS. 19a and 19b, no fluorescence was detected in the control cells (PBS injected) while Cy5.5 was detected in blood cells, especially lymphocytes and monocytes, of the miRNA trigger-injected mice.

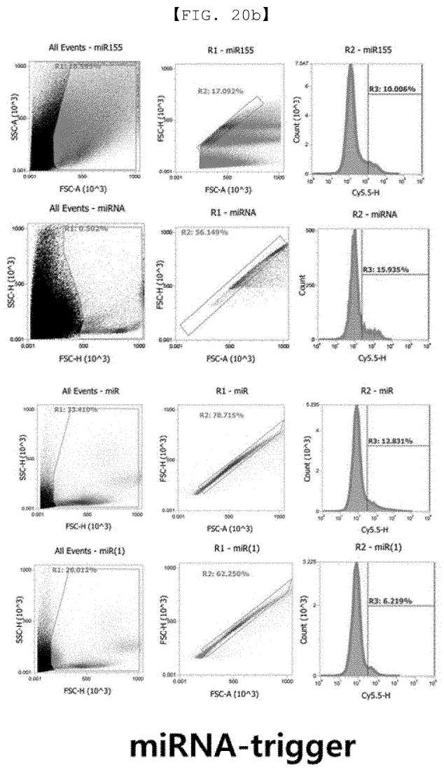

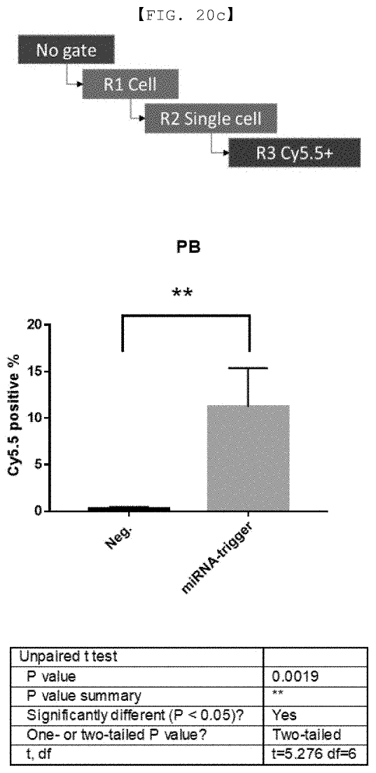

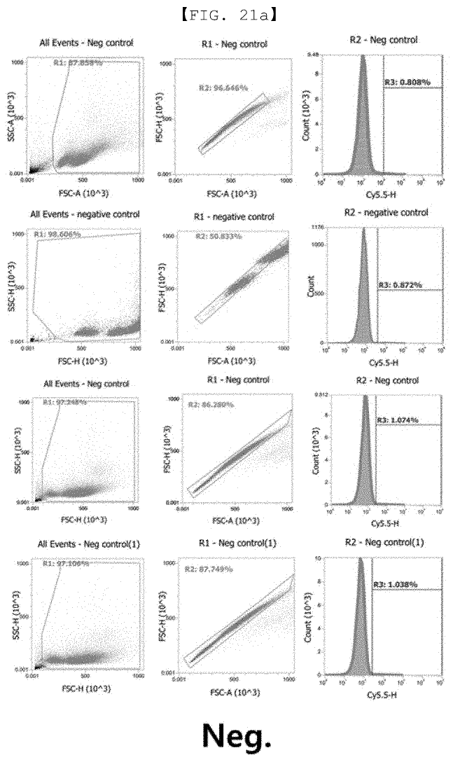

The cells in peripheral blood sample were gated through three steps (step 1: debris removal, step 2: selection of single blood cell population, step 3: Cy5.5 detection) to select single blood cells of quality. Through FACS, blood cells retaining Cy5.5 were identified. The results are depicted in FIGS. 20a to 20c. As shown in FIGS. 20a to 20c, the miRNA trigger injected by the in-vivo system was actually delivered into blood cells and the Cy5.5 was detected at a rate of 6.2-15.9%, based on the total number of the single blood cells.

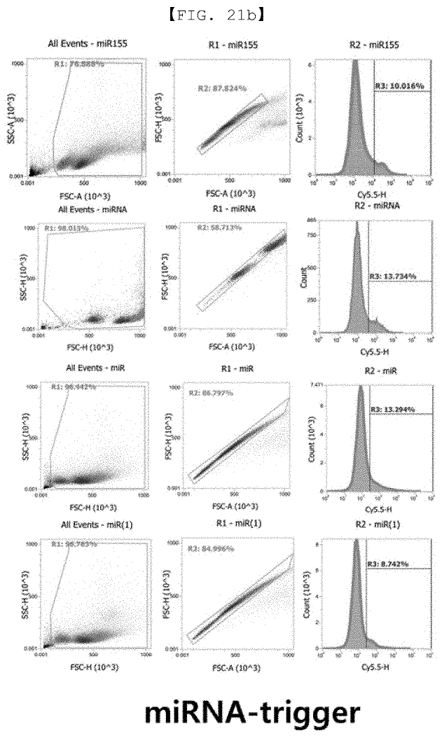

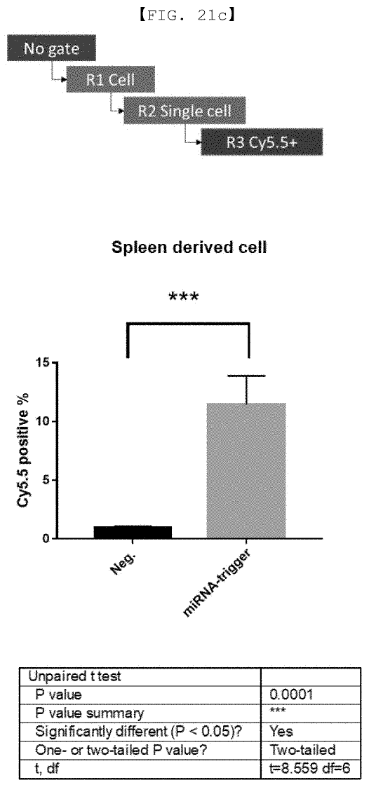

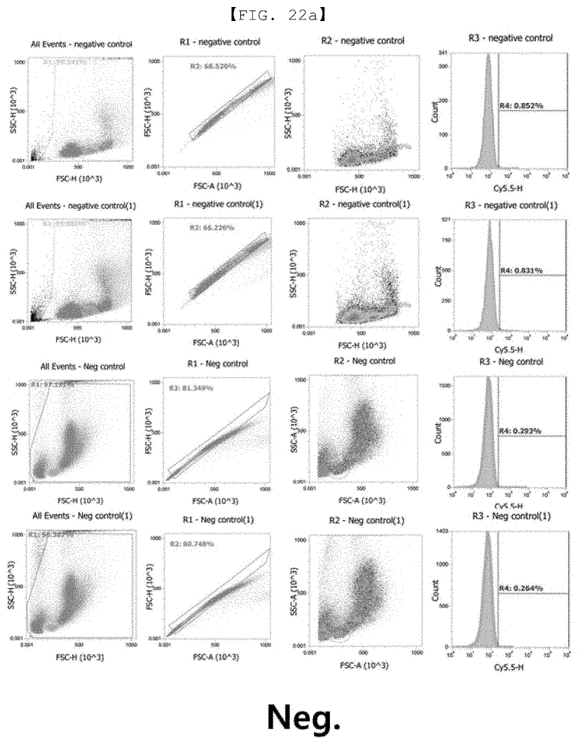

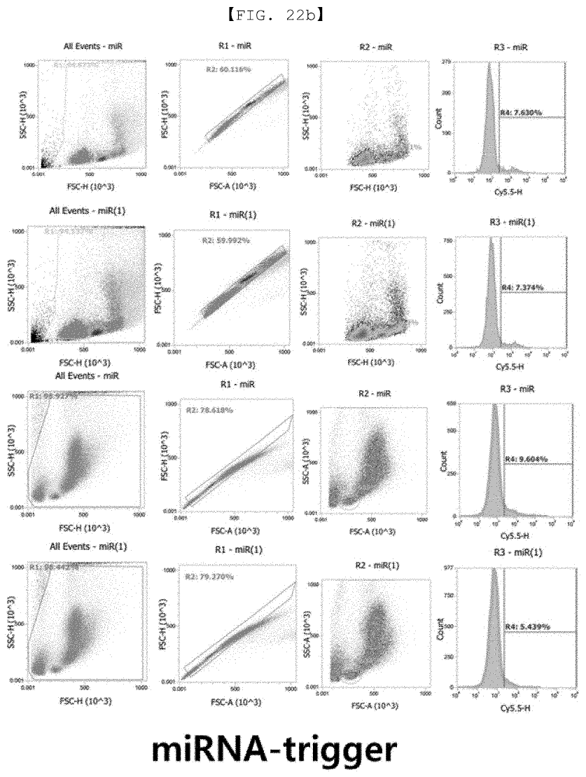

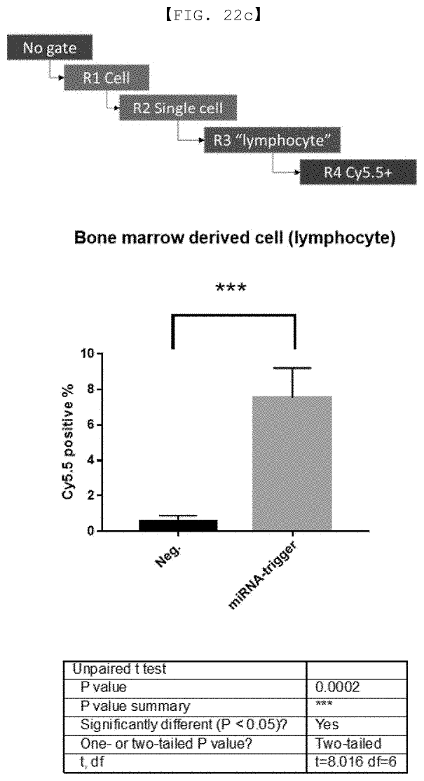

Spleen-derived hematopoietic cells and bone marrow hematopoietic cells of mice were gated in the foregoing manner and Cy5.5 was detected. The results are depicted in FIGS. 21 and 22. As shown in FIGS. 21a to 21c, 8.7-13.7% of total spleen-derived hematopoietic cells expressed Cy5.5. As in peripheral blood cells, Cy5.5 was detected mainly in lymphocytes and monocytes of the spleen-derived hematopoietic cells.

In addition, as shown in FIGS. 22a to 22c, the rate of bone marrow cells expressing Cy5.5 was 1.0-2.0% and relatively low, with the predominant expression of Cy5.5 in lymphocytes. Thus, lymphocytes were gated through 4 steps. As a result, Cy5.5 was detected from 5.4-9.6% of the cell.

From the data, it was understood that even at 4 days after intravenous injection into B6 mice, the miRNA trigger was detected in main organs and hematopoietic organs and intracellularly delivered into blood cells such as lymphocytes and monocytes.

As shown in FIGS. 21 and 22, the miRNA trigger according to an embodiment could be delivered into single cells of peripheral blood.

### Example 7. Therapeutic Effect of miRNA Trigger on Blood Cancer

### 7-1. Selection of AML-Specific miRNA

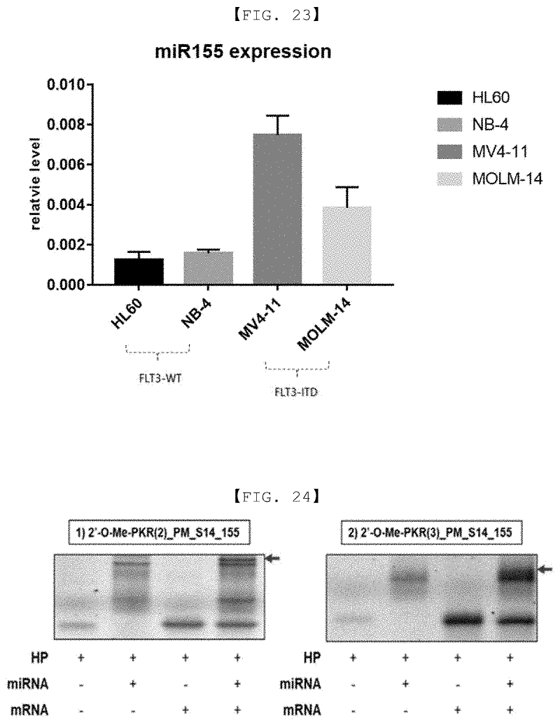

In this Example, the miRNA trigger technology was applied to the therapy of AML (acute myeloid leukemia). In this regard, various AML cell lines were established and selection was made of miRNAs that are expressed specifically in AML cells. FLT3-ITD mutation, which is observed in about 25% of AML patients, was selected as a target disease. MV4-11 and MOLM-14 cells, both having FLT3-ITD mutation, were acquired while NB-4 and HL60 cells, both exhibiting FLT3-WT, were used as controls. In order to examine whether the expression of miR-155 was increased in FLT3-ITD mutated AML cells, miR-155 levels were quantitated using qRT-PCR. The results are depicted in FIG. 23. As seen in FIG. 23, the FLT3-ITD mutated AML cells MV4-11 and MOLM-14 both had an elevated expression level of miR-155.

### Example 7-2. Preparation of miR-155-Specific Trigger

The hairpin miRNA triggers HP_PKR(2)_155 (2′-O-Me-PKR(2)_PM_S14_155) and HP_PKR(3)_155 (2′-O-Me-PKR(3)_PM_S14_155) which could both bind to miR-155 and target the pkr gene were prepared in a similar manner as in Reference Example 1-2. Every nucleotide in the HPs used in this Example was chemically modified with 2′-OMe, and all of the nucleotides in the portions capable of binding to the miRNA were complementary to those in the miRNA sequence (perfect match; PM), with the stem being 14 nt long (S14). Sequences of the HP, miRNA, and mRNA are listed in Table 18, below.

[symbol]

| | | SEQ |
|---|---|---|
| Oligo | | ID |
| name | Sequence (5′->3′) | NO |
| HPPKR(2) | mUmAmUmUmUmCmUmCmAmUmUmCmCmCmAmAm | 70 |
| _155 | CmCmCmCmUmAmUmCmAmCmGmAmUmUmAmGm | |
| | CmAmUmUmAmAmGmGmGmAmAmUmGmAmGmAm | |
| | AmAmUmA | |
| HP_PKR | mUmAmGmCmUmGmUmAmCmUmUmCmAmAmAmAm | 71 |
| (3)_1 | CmCmCmCmUmAmUmCmAmCmGmAmUmUmAmGm | |
| | CmAmUmUmAmAmUmUmGmAmAmGmUmAmCmAm | |
| | GmCmUmA | |
| miR-155 | UUAAUGCUAAUCGUGAUAGGGGUU | 79 |
| PKRmRNA | CAAUAAUGGGAAGGAAGGGAAUGA | 73 |
| (2) | GAAAUAUUAAAUUCUG | |
| PKRmRNA | GUAUUGAAAACAAUUGAAGUACAGC | 74 |
| (3) | UAAAUGUAAUAACG | |

In a buffer (1×PBS, 137 mM NaCl, 10 mM PO4, 2.7 mM KCl, 5 mM MgCl₂; pH 7.4), the hairpin miRNA triggers, miR-155 and/or the pkr mRNA were each added at a concentration of 100 nM and incubated at 37° C. for 1 hour. The mixtures were loaded to a PAGE gel and run to examine whether the hairpin miRNA triggers bind to the miRNA and the mRNA. The results are depicted in FIG. 24. As shown in FIG. 24, large sizes of bands appeared in the presence of the miRNA alone or in combination of the mRNA. Thus, it was confirmed that the miRNA triggers bound to the miRNA to open their hairpin structures and then hybridized with the mRNAs. Even when the mRNAs were added, no size-increased bands were not detected in the absence of the miRNA, indicating that the hairpin miRNA trigger does not hybridize with the mRNA unless binding to the miRNA.

### Example 7-3. Selection of Gene Inducing AML Apoptosis

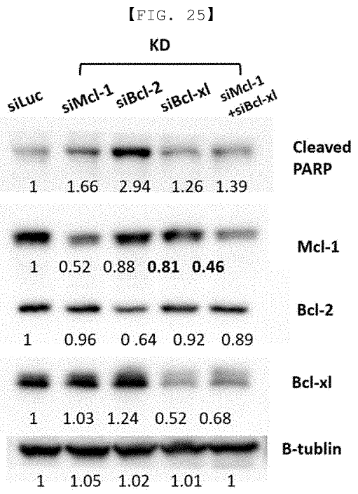

Selection was Made of a Target Gene of the miRNA trigger to induce apoptosis in blood cancer cell lines. In this regard, the Bcl-2 family genes, which are anti-apoptotic genes, were knocked down by transfecting siRNAs (siMcl-1, siBcl-2, and siBcl-xL; Table 14) against the genes into HL-60 cells, followed by western blotting to measure protein levels of the apoptotic marker (cleaved PARP). The results are depicted in FIG. 25.

As can be seen in FIG. 25, when bcl-2 among the Bcl-2 family genes was knocked down (siBcl-2) in the blood cancer cell line HL-60, the expression of the apoptotic marker cleaved PARP was upregulated, demonstrating potent apoptosis induction.

### Example 7-4. Therapeutic Agent Usable in Combination with miRNA Trigger

In this Example, investigation was made of agents that can exhibit excellent therapeutic effects on AML when used in combination with a miRNA trigger capable of binding miR-155.

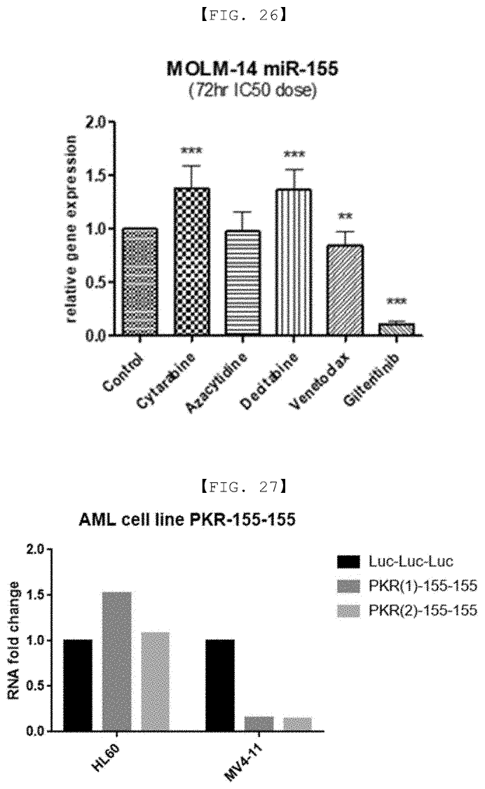

The FLT3-ITD mutated AML cell line (NOLM-14) was treated with an IC₅₀ dose of cytarabine, azacitidine, decitabine, venetoclax, or gilteritinib, which is used as standard therapeutic agents for AML, at 37° C. for 72 hours, followed by measuring expression levels of miR-155. The results are given in FIG. 26. As shown in FIG. 26, the cytotoxic agent cytarabine and the hypomethylating agents azacytidine and decitabine did not repress miR-155 express whereas a remarkably decrease in miR-155 expression was induced by the FLT3 inhibitor gilteritinib and the Bcl-2 inhibitor venetoclax.

From the result, it was understood that cytarabine, azacitidine, and decitabine did not deplete miR-155 and thus could be used as agents which could be used, in combination with a miRNA trigger capable of binding miR-155, to exhibit excellent AML treatment effects.

### Example 7-5. Action of Linear miRNA Trigger in AML Cell Line

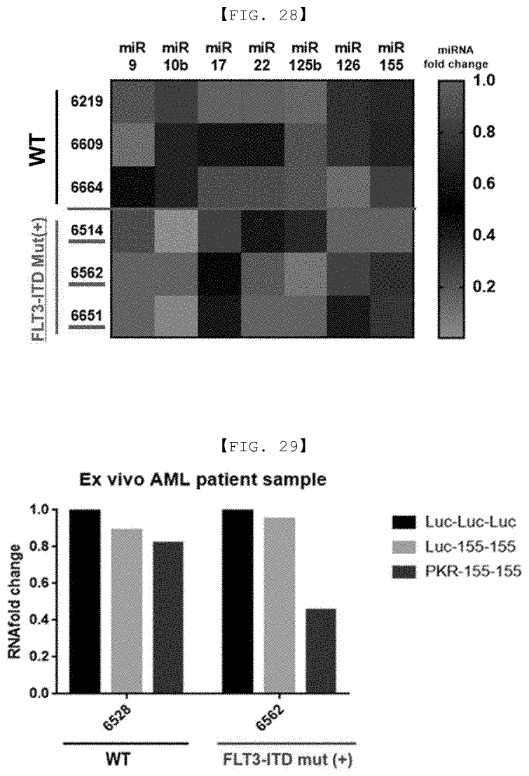

The linear miRNA trigger (PKR-155-155) that could bind to miR-155 and target pkr gene was prepared and then transfected into the FLT3-ITD mutated cell line MV4-11 and the FLT3-WT cell line HL60 as a control, followed by measuring expression levels of the target gene (pkr). The results are depicted in FIG. 27. Sequences are listed in Table 18 for the linear miRNA triggers and in Table 9 for the control Luc-Luc-Luc. Every nucleotide of the triggers and the control were chemically modified with 2′-OMe.

TABLE 18

| | | SEQ |
|---|---|---|
| Oligo | Sequence | ID |
| name | (5′-3′) | NO |
| PKR(1)- | mUmUmAmUmGmUmUmUmCmAmGmGmUmUmCmAmGmGmG | 75 |
| 155-155 | mGmGmAmUmUmAmUmGmUmUmUmCmAmGmGmUmUmCmA | |
| | mGmGmGmGmGmAmUmAmUmGmUmGmAmGmGmCmAmGmA | |
| | mGmAmAmCmGmA | |
| PKR(2)- | mUmUmAmUmGmUmUmUmCmAmGmGmUmUmCmAmGmGmG | 76 |
| 155-155 | mGmGmAmUmUmAmUmGmUmUmUmCmAmGmGmUmUmCmA | |
| | mGmGmGmGmGmAmUmAmUmUmUmCmUmCmAmUmUmCmC | |
| | mCmUmUmCmCmU | |
| luc_ | mUmUmAmUmGmUmUmUmCmAmGmGmUmUmCmAmGmGmG | 7 7 |
| 155-155 | mGmGmAmUmUmAmUmGmUmUmUmCmAmGmGmUmUmCmA | |
| | mGmGmGmGmGmAmUmCmGmAmAmGmUmAmCmUmCmAmG | |
| | mCmGmUmAmAmG | |

As shown in FIG. 27, PKR-155-155 successfully knocked down PKR in MV4-11, but no effects of the miRNA triggers were observed in HL60. These results indicate that the expression of a target gene can be successfully regulated by a miRNA trigger in AML cell lines.

### Example 7-5. Assay for Efficacy of miRNA Trigger in AML Patient-Derived Primary Cells

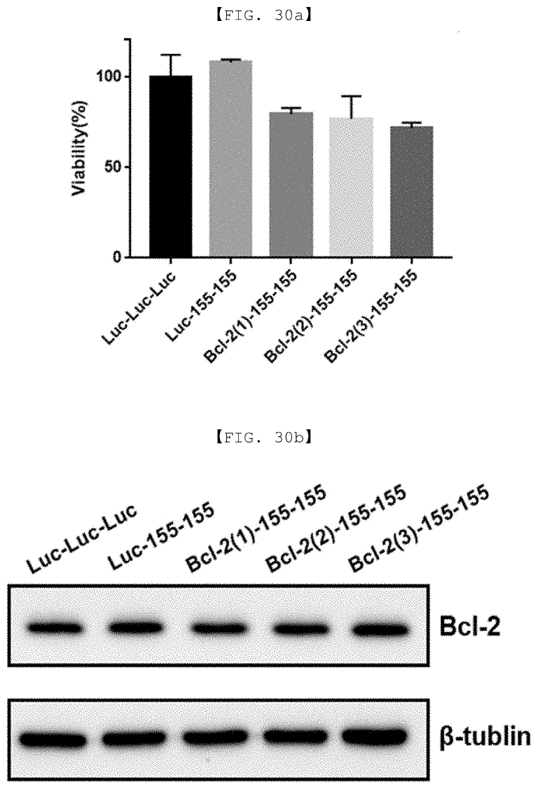

Blood samples taken from the bone marrows of AML patients were quantitated for expression levels of various miRNAs and compared. Myeloblasts including monocytes, present in the buffy coat layers separated from 10 blood samples taken from 10 patients, were transferred to a culture system under the same condition as that for cell lines and cultured in a lab scale. Among them, 7 patent samples that had high cell activity (cells that did not die, but were growing) were selected for RNA extraction. Expression levels of miR-9, miR-10b, miR-17, miR-22, miR-125b, miR-126, and miR-155 were quantitated using stem-loop qRT-PCR, and the results are depicted in FIG. 28. In stem-loop qRT-PCR, a ramping process is employed to reconstitute a miRNA-specific RNA into a stem loop structure after extraction of total RNA from cells. cDNA was synthesized from the extracted RNA, using a stem-loop primer or a U6 primer, followed by conducting qPCR with gene-specific primers. U6 was used as a comparative group. Gene-specific stem loop reverse transcription (RT) primers and qPCR primers used in the stem loop qRT-PCR are as follows.

TABLE 19

| | | | SEQ |
|---|---|---|---|
| Target | Primer | | ID |
| name | name | Sequence (5′-> 3′) | NO |
| U6 | RT Primer | CGCTTCACGAATTTGCGTGTCAT | 78 |
| | Forward | GCTTCGGCAGCACATATACTAAAAT | 79 |
| | Primer | | |
| | Reverse | CGCTTCACGAATTTGCGTGTCAT | 80 |
| | Primer | | |
| miR- | RT Primer | GAAAGAAGGCGAGGAGCAGATCGAGGAAG | 81 |
| 9 | | AAGACGGAAGAATGTGCGTCTCGCCTTCT | |
| | | TTCTCATACAG | |
| | Forward | GCGTCTTTGGTTATCTAGCTG | 82 |
| | Primer | | |
| | Reverse | CGAGGAAGAAGACGGAAGAAT | 83 |
| | Primer | | |
| miR- | RT Primer | GAAAGAAGGCGAGGAGCAGATCGAGGAAG | 84 |
| 10b | | AAGACGGAAGAATGTGCGTCTCGCCTTCT | |
| | | TTCCACAAATT | |
| | Forward | GCTACCCTGTAGAACCGAAT | 85 |
| | Primer | | |
| | Reverse | CGAGGAAGAAGACGGAAGAAT | 86 |
| | Primer | | |
| miR- | RT Primer | GAAAGAAGGCGAGGAGCAGATCGAGGAAG | 87 |
| 17 | | AAGACGGAAGAATGTGCGTCTCGCCTTCT | |
| | | TTCCTACCTGC | |
| | Forward | GCCAAAGTGCTTACAGTGCA | 88 |
| | Primer | | |
| | Reverse | CGAGGAAGAAGACGGAAGAAT | 89 |
| | Primer | | |
| miR- | RT Primer | GAAAGAAGGCGAGGAGCAGATCGAGGAAG | 90 |
| 22 | | AAGACGGAAGAATGTGCGTCTCGCCTTCT | |
| | | TTCACAGTTCT | |
| | Forward | GCAAGCTGCCAGTTGAAGAA | 91 |
| | Primer | | |
| | Reverse | CGAGGAAGAAGACGGAAGAAT | 92 |
| | Primer | | |
| miR- | RT Primer | GAAAGAAGGCGAGGAGCAGATCGAGGAAG | 93 |
| 125b | | AAGACGGAAGAATGTGCGTCTCGCCTTCT | |
| | | TTCTCACAAGT | |
| | Forward | GCGTCGTACCGTGAGTAATAA | 94 |
| | Primer | | |
| | Reverse | CGAGGAAGAAGACGGAAGAAT | 95 |
| | Primer | | |
| miR- | RT Primer | GAAAGAAGGCGAGGAGCAGATCGAGGAAG | 96 |
| 126 | | AAGACGGAAGAATGTGCGTCTCGCCTTCT | |
| | | TTCCGCATTAT | |
| | Forward | GTCCCTGAGACCCTAACTT | 97 |
| | Primer | | |
| | Reverse | CGAGGAAGAAGACGGAAGAAT | 98 |
| | Primer | | |
| miR- | RT Primer | GAAAGAAGGCGAGGAGCAGATCGAGGAAG | 99 |
| 155 | | AAGACGGAAGAATGTGCGTCTCGCCTTCT | |
| | | TTCAACCCCTA | |
| | Forward | GCGGTTAATGCTAATCGTGATA | 100 |
| | Primer | | |
| | Reverse | CGAGGAAGAAGACGGAAGAAT | 101 |
| | Primer | | |

As shown in FIG. 28, three of the six patient samples were obtained from the patients who had been identified to have FLT3-ITD mutation, and were higher in miR-155 expression level than AML patient samples in the FLT3-WT group.

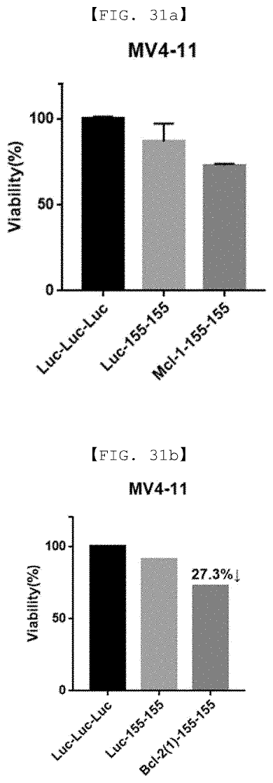

To the 6528-sample taken from FLT3-WT patients and the 6562-sample taken from FLT3-ITD mutated patients were transfected the linear miRNA trigger (PKR-155-155; Table 18) which could bind to miR-155 and target pkr and the control linear miRNA triggers (Luc-155-155, Luc-luc-luc; Tables 9 and 18) which target Luc, followed by quantitation of pkr expression levels. The results are depicted in FIG. 29.

As shown in FIG. 29, PKR-155-155 decreased the expression of pkr specifically in the 6562-sample, but PKR was reduced to a lesser degree in the FLT3-WT 6528-sample due to a low expression level of miR-155.

From the results, it was understood that when applied to FLT3-ITD-mutated AML patients, a miRNA trigger capable of binding to miR-155 can regulate the expression of the target gene in a specific manner for FLT3-ITD-mutated AML patients.

### Example 7-6. Effect of miRNA Trigger Targeting bcl-2

Five linear miRNA triggers which could each bind to miR-155 and target bcl-2 gene were prepared in the same manner as in Reference Example 1. The five linear miRNA triggers included respective portions capable of binding complementarily to bcl-2 gene, with a difference among the portions. Sequences are listed in Table 20 for the linear miRNA triggers, in Tables 18 and 19 for the controls Luc-Luc-Luc and luc-155-155, respectively, and in Table 13 for siRNAs. Every nucleotide of the triggers and the controls were chemically modified with 2′-OMe.

TABLE 20

| | | SEQ |
|---|---|---|
| Oligo | Sequence | ID |
| name | (5′-3′) | NO |
| Bcl-2 | mUmUmAmUmGmUmUmUmCmAmGmGmUmUmCmAm | 102 |
| (1)_155_ | GmGmGmGmGmAmUmUmAmUmGmUmUmUmCmAmG | |
| 155 | mGmUmUmCmAmGmGmGmGmGmAmUmUmGmCmCm | |
| | UmGmAmAmGmAmCmUmGmUmUmAmA | |
| Bcl-2 | mUmUmAmUmGmUmUmUmCmAmGmGmUmUmCmAm | 103 |
| (2)_155_ | GmGmGmGmGmAmUmUmAmUmGmUmUmUmCmAmG | |
| 155 | mGmUmUmCmAmGnGmGmGmGmAmAmUmGmCmCm | |
| | AmCmAmGmAmGmUmUmAmUmUmCmC | |
| Bcl-2 | mUmUmAmUmGmUmUmUmCmAmGmGmUmUmCmAm | 104 |
| (3)_155_ | GmGmGmGmGmAmUmUmAmUmGmUmUmUmCmAmG | |
| 155 | raGmUmUmCmAmGmGmGmGmGmAmAmUmAmCmAm | |
| | CmUmAmUmUmUmGmUmGmAmGmCmA | |

The five linear miRNA triggers and the positive siRNA (siBcl-2) capable of binding bcl-2 were transfected to the FLT3-mutated AML cell line MOLM-14 which was then measured for cell viability. The results are depicted in FIG. 30a.

FIG. 30a is a graph of cell viability (%) after transfection of the AML cell line MOLM-14 with the linear miRNA triggers capable of binding to miR-155 and to various site of Bcl-2.

As shown in FIG. 30a, the degree of miRNA trigger-induced apoptosis varied depending on the binding site of the bcl-2 gene to be targeted. The transfection of the linear miRNA triggers according to an embodiment was observed to lead to an apoptotic effect on blood cancer cells.

In addition, the miRNA trigger-transfected cells were measured for protein expression levels of the target Bcl-2 and the apoptotic marker cleaved PARP through western blotting, and the results are given in FIG. 30b. As shown in FIG. 30b, the miRNA triggers downregulated the protein expression of Bcl-2.

### Example 7-7. Effect of miRNA Trigger Targeting mcl-1

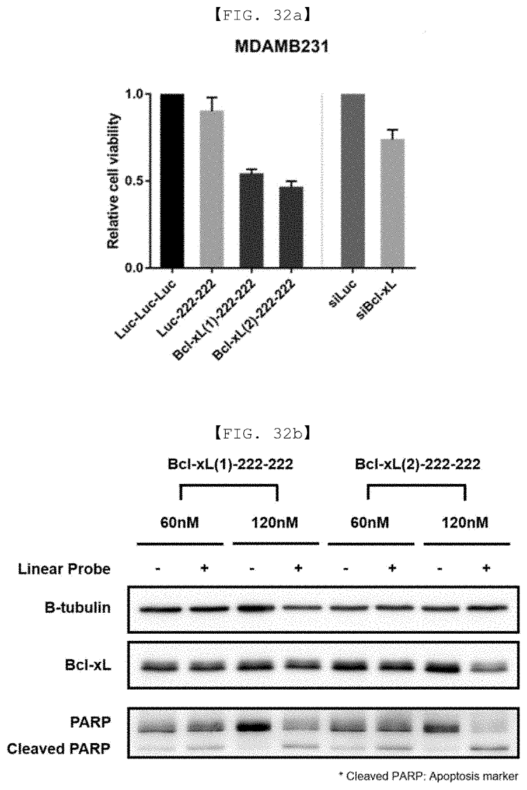

A linear miRNA trigger (Mcl-1_155_155 or Bcl-2_155_155) which could bind to miR-155 and target mcl-1 or bcl-2 gene was prepared and transfected into the FLT3 mutated AML cell line MV4-11 which was then measured for cell viability. The results are depicted in FIGS. 31a and 31b. Sequences are listed in Tables 19 and 21 for the linear miRNA triggers prepared above, and in Tables 18 and 19 for the controls Luc-Luc-Luc and luc-155-155, respectively. Every nucleotide of the triggers and the controls were chemically modified with 2′-OMe.

TABLE 21

| | | SEQ |
|---|---|---|
| Oligo | Sequence | ID |
| name | (5′-3′) | NO |
| Mcl-1_ | mUmUmAmUmGmUmUmUmCmAmGmGmUmUmCmAmGmG | 105 |
| 155_155 | mGmGmGmAmUmUmAmUmGmUmUmUmCmAmGmGmUmU | |
| | maCmAmGmGmGmGmGmAmCmCmGmAmAmCmUmAmCmG | |
| | mUmAmGmC | |

As shown in FIGS. 31a and 31b, the miRNA trigger targeting mcl-1 or bcl-2 can induce MV4-11 to undergo apoptosis.

### Example 8. Assay for Therapeutic Effect of miRNA Trigger on Breast Cancer

### Example 8-1. Effect of Linear miRNA Trigger Targeting Bcl-xL

Two linear miRNA triggers (Bcl-xL(1)-222-222 and Bcl-xL(2)-222-222) which could each bind to miR-222 and knock down bcl-xL gene were prepared in the same manner as in Reference Example 1. The two linear miRNA triggers included respective portions capable of complementarily binding to bcl-xL gene, but with a difference between the portions. Sequences are listed in Table 22 for the linear miRNA triggers prepared above and in Tables 22 and 9 for the controls Luc-Luc-Luc and luc-222-222, respectively. Every nucleotide of the triggers and the controls were chemically modified with 2′-OMe.

TABLE 22

| | | SEQ |
|---|---|---|
| Oligo | Sequence | ID |
| name | (5′-3′) | NO |
| bcl-xL(1)_ | mAmCmCmCmAmGmUmAmGmCmCmAmGm | 106 |
| 222_222 | AmUmGmUmAmGmCmUmAmCmCmCmAm | |
| | GmUmAmGmCmCmAmGmAmUmGmUmAm | |
| | GmCmUmUmCmUmCmCmUmUmCmCmUm | |
| | GmCmCmCmUmUmCmCmU | |
| bcl-xL(2)_ | mAmCmCmCmAmGmUmAmGmCmCmAm | 107 |
| 222_222 | GmAmUmGmUmAmGmCmUmAmCmCmCm | |
| | AmGmUmAmGmCmCmAmGmAmUmGmUm | |
| | AmGmCmUmUmAmCmCmUmGmCmCmAm | |
| | GmCmCmUmCmCmUmU | |
| luc_222- | mAmCmCmCmAmGmUmAmGmCmCmAm | 108 |
| 222 | GmAmUmGmUmAmGmCmUmAmCmCmCm | |
| | AmGmUmAmGmCmCmAmGmAmUmGmUm | |
| | AmGmCmUmUmCmGmAmAmGmUmAmCm | |
| | UmCmAmGmCmGmUmAmAmG | |

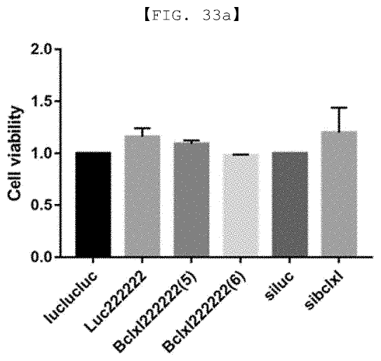

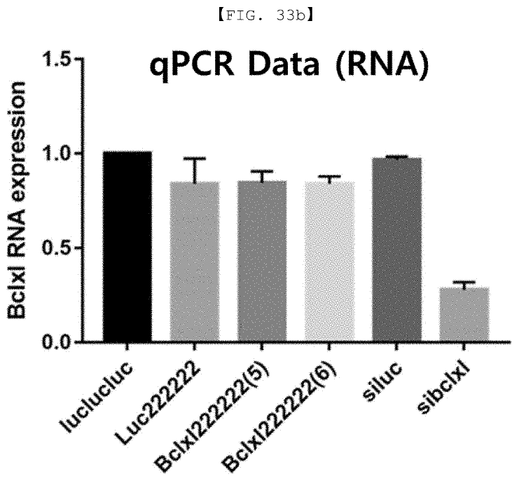

The seven linear miRNA triggers prepared above and the positive control siRNA(siBcl-xL) capable of binding to bcL-xL were transfected into the breast cancer cell line MDA-MB-231 in which miR-222 is overexpressed. Cell viability was measured and is depicted in FIG. 32a.

As shown in FIG. 32a, the degree of miRNA trigger-induced apoptosis varied depending on the binding site of the bcL-xL gene to be targeted. The two linear miRNA triggers that can effectively kill breast cancer cells (in FIG. 30a, (1) and (2) with better reproducibility) were selected.

The cells transfected with the selected miRNA triggers were measured for protein expression levels of the target Bcl-xL and the apoptotic marker cleaved PARP through western blotting, and the results are depicted in FIG. 32b. As shown in FIG. 32b, the miRNA triggers decreased the expression level of Bcl-xL protein levels and decreased the expression level of the apoptotic marker cleaved in a dose-dependent manner.

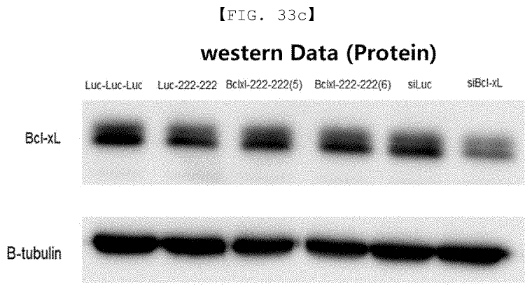

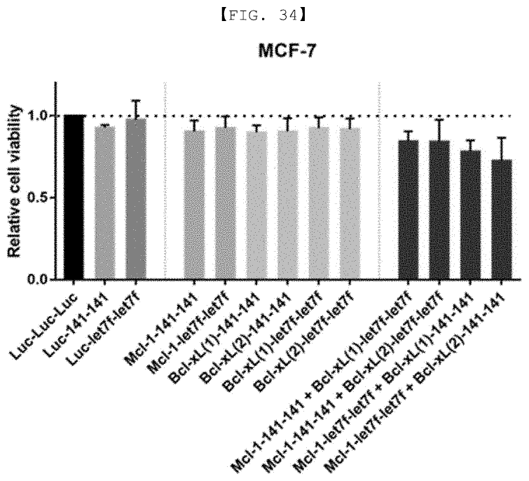

The selected linear miRNA triggers (bcl-xL(1)_222_222 and bcl-xL(2)_222_222) were transfected into the breast cancer cell line MDA-MB-453 to which miR-222 is not endogenous. Cell viability, and the mRNA and protein expression levels of the target gene (bcl-xL) were measured and the results are depicted in FIGS. 33a to 33c. As opposed to MDA-MB-231, MDA-MB-453, which does not express miR-222, did not significantly change in cell viability and the mRNA and protein expression levels of the target bcl-xL even when transfected with the linear miRNA triggers according to an embodiment.

### Example 8-2. Effect of Linear miRNA Trigger Targeting bcl-xL or mcl-1

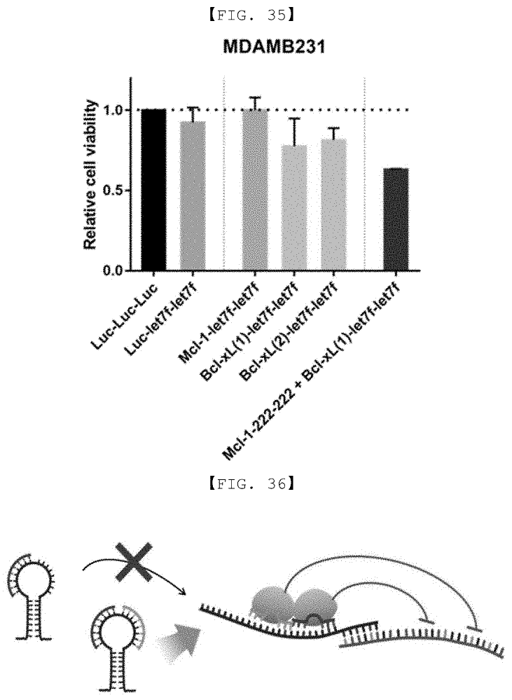

Linear miRNA triggers that could bind to miR-222 and target bcl-xL and mcl-1, respectively, were prepared and transfected alone or in combination into the breast cancer cell line MDA-MB-231. Cell viability was measured and the measurements are depicted in FIG. 34. siRNAs against Mcl-1 and Bcl-xL were used as controls. Sequences are listed in Table 23 for the linear miRNA trigger prepared above, in Tables 22 and 9 for the controls Luc-Luc-Luc and luc-222-222, respectively, and in Table 13 for the siRNAs. Every nucleotide of the triggers and the controls was chemically modified with 2′-OMe.

TABLE 23

| | | SEQ |
|---|---|---|
| Oligo | Sequence | ID |
| name | (5′-3′) | NO |
| Mel-1_ | mAmCmCmCmAmGmUmAmGmCmCmAmGmAmUm | 109 |
| 222_222 | GmUmAmGmCmUmAmCmCmCmAmGmUmAmGm | |
| | CmCmAmGmAmUmGmUmAmGmCmUmCmCmGm | |
| | AmAmCmUmAmCmGmUmAmGmC | |

As shown in FIG. 34, the transfection of the bcl-xL gene-targeting miRNA trigger brought about a remarkable apoptotic effect, but the additional introduction of the mcl-i-targeting miRNA trigger did not further increase the apoptotic effect.

### Example 8-3. Effect of miRNA Trigger Capable of Binding to miR-141 or let7f

Linear miRNA triggers that could bind to miR-141 or let7f and target bcl-xL or mcl-1 were prepared and transfected alone or in combination into the breast cancer cell line MCF-7 which was then measured for cell viability. The measurements are given in FIG. 34. Sequences of the miRNA triggers and the controls used in this Example are listed in Tables 24, 9, and 13. Every nucleotide of the triggers and controls was chemically modified with 2′-OMe.

TABLE 24

| | | SEQ |
|---|---|---|
| Oligo | Sequence | ID |
| name | (5′-3′) | NO |
| Luc_141_ | mCmCmAmUmCmUmUmUmAmCmCmUmCmAmCmAmGm | 50 |
| 141 | UmGmUmUmAmCmCmAmUmCmUmUmUmAmCmCmUmC | |
| | mAmCmAmGmUmGmUmUmAmUmCmGmAmAmGmUmAm | |
| | CmUmCmAmGmCmGmUmAmAmG | |
| Mcl-1_ | mCmCmAmUmCmUmUmUmAmCmCmUmCmAmCmAmGm | 110 |
| 141_141 | UmGmUmUmAmCmCmAmUmCmUmUmUmAmCmCmUmC | |
| | mAmCmAmGmUmGmUmUmAmCmCmGmAmAmCmUmAm | |
| | CmGmUmAmGmC | |
| Luc_ | mUmCmCmAmUmCmAmUmUmAmCmCmGmCmGmCmAm | 51 |
| 200c_ | GmUmAmUmUmAmUmCmCmAmUmCmAmUmUmAmCmC | |
| 200c | mGmCmGmCmAmGmUmAmUmUmAmUmCmaGmAmAmGm | |
| | UmAmCmUmCmAmGmCmGmUmAmAmG | |
| Mcl-1_ | mUmCmCmAmUmCmAmUmUmAmCmCmGmCmGmCmAm | 111 |
| 200c_ | GmUmAmUmUmAmUmCmCmAmUmCmAmUmUmAmCmC | |
| 200c | mGmCmGmCmamGmUmAmUmUmAmCmCmGmAmAmCm | |
| | UmAmCmGmUmAmGmC | |
| luc_ | mAmAmCmUmAmUmAmCmAmAmUmGmAmAmCmUmAm | 112 |
| let7f_ | CmCmUmCmAmAmAmCmUmAmUmAmCmAmAmUmGmA | |
| let7f | mAmCmUmAmCmCmUmCmAUmCmGmAmAmGmUmAmC | |
| | mUmCmAmGmCmGmUmAmAmG | |
| Mcl-1_ | mAmAmCmUmAmUmAmCmAmAmUmGmAmAmCmUmAm | 113 |
| let7f_ | CmCmUmCmAmAmAmCmUmAmUmAmCmAmAmUmGmA | |
| let7f | mAmCmUmAmCmCmUmCmAmCmCmGmAmAmCmUmAm | |
| | CmGmUmAmGmC | |
| bcl- | mAmAmCmUmAmUmAmCmAmAmUmGmAmAmCmUmAm | 114 |
| xL(1)_ | CmCmUmCmAmAmAmCmUmAmUmAmCmAmAmUmGmA | |
| let7f_ | mAmCmUmAmCmCmUmCmAmUmCmUmCmCmUmUmCm | |
| let7f | CmUmGmCmCmCmUmUmCmCmU | |
| bel- | mAmAmCmUmAmUmAmCmAmAmUmGmAmAmCmUmAm | 115 |
| xL(2)_ | CmCmUmCmAmAmAmCmUmAmUmAmCmAmAmUmGmA | |
| let7f_ | mAmCmUmAmCmCmUmCmAmUmAmCmCmUmGmCmCm | |
| let7f | AmGmCmCmUmCmCmUmU | |
| Bcl- | mCmCmAmUmCmUmUmUmAmCmCmUmCmAmCmAmGm | 116 |
| xL(1)- | UmGmUmUmAmCmCmAmUmCmUmUmUmAmCmCmUmC | |
| 141-141 | mAmCmAmGmUmGmUmUmAmUmCmUmCmCmUmUmCm | |
| | CmUmGmCmCmCmUmUmCmCmU | |
| Bcl- | mCmCmAmUmCmUmUmUmAmCmCmUmCmAmCmAmGm | 117 |
| xL(2)- | UmGmUmUmAmCmCmAmUmCmUmUmUmAmCmCmUmC | |
| 141-141 | mAmCmAmGmUmGmUmUmAmUmAmCmCmUmGmCmCm | |
| | AmGmCmCmUmCmCmUmU | |

As shown in FIG. 34, the miRNA triggers exhibited excellent apoptotic effect on breast cancer cells, with more intensive apoptosis detected in the group treated with both the mcl-1-targeting miRNA trigger and the bcl-xL-targeting miRNA trigger.

### Example 8-4. Effect of nM Trigger Targeting miR-222 or let7f

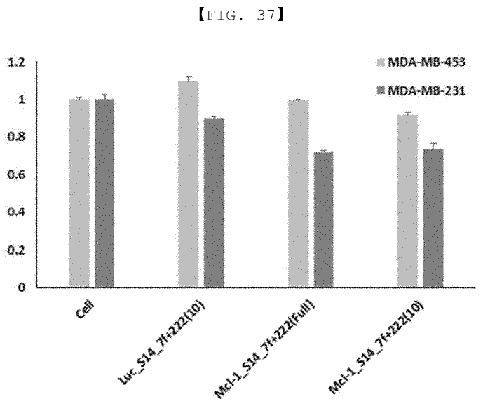

Linear miRNA triggers that could bind to miR-141 or let7f and target Bcl-xL or Mcl-1 were prepared and transfected alone or in combination into the breast cancer cell line MDA-MB-231 which was then measured for cell viability. The measurements are given in FIG. 35. Sequences of the miRNA triggers and the controls used in this Example are listed in Tables 9 and 24. Every nucleotide of the triggers and controls was chemically modified with 2′-OMe.

As shown in FIG. 35, the miRNA triggers according to an embodiment induced a high level of apoptosis in MDA-MB-231, and synergistically increased the apoptotic effect when used in combination with the mcl-i-targeting miRNA trigger.

### Example 9. Hairpin MiRNA Trigger Capable of Binding to Two Types of miRNAs

Hairpin miRNA triggers designed to open their hairpin structures upon binding to two different miRNAs were prepared in a same manner as in Example 2. The hairpin miRNA triggers prepared in this Example have the following structure:

[5′-portion capable of binding to mRNA of target gene (T*)-portion capable of binding to miRNA(1)(mi(1)*)-spacer(TT)-portion capable of binding to miRNA(2)(mi(2)*)-portion capable of binding toT* (T)-3′].

Hairpin miRNA triggers that could bind to both Let-7f and miR-222 and target mcl-1 were synthesized in Bioneer Inc., purified through HPLC, and stored until subsequent experiments. Every nucleotide was modified with 2′-O-methyl. Sequences of the miRNA triggers are listed in Table 25. In the table below, (10) means a hairpin in which only 10 mers from the 5′ of the miRNA binds to the loop and (full) means a hairpin in which the full sequence of the miRNA binds to the loop.

TABLE 25

| | | SEQ |
|---|---|---|
| Oligo | Sequence | ID |
| name | (5′->3′) | NO |
| HP_Luc_7f+ | mUmCmGmAmAmGmUmAmCmUmCmAmGmCmAmAmC | 118 |
| 222(10) | mUmAmUmAmCmAmAmAmCmCmCmAmGmUmAmGmC | |
| | mGmCmUmGmAmAmCmAmCmUmCmUmCmA | |
| HP_Mcl- | mCmCmGmAmAmCmUmAmCmGmUmAmGmCmAmAmC | 119 |
| 1_7f+222 | mUmAmUmAmCmAmAmUmCmUmAmCmUmAmCmCmU | |
| (full) | mCmAmAmCmCmCmAmGmUmAmGmCmCmAmGmAmU | |
| | mGmUmAmGmCmUmGmCmUmAmCmGmUmAmGmUmU | |
| | mCmGmG | |
| HP_Mcl- | mCmCmGmAmAmCmUmAmCmGmUmAmGmCmAmAmC | 120 |
| 1_7f+ | mUmAmUmAmCmAmAmAmCmCmCmAmGmUmAmGmC | |
| 222(10) | mGmCmUmAmCmGmUmAmGmUmUmCmGmG | |

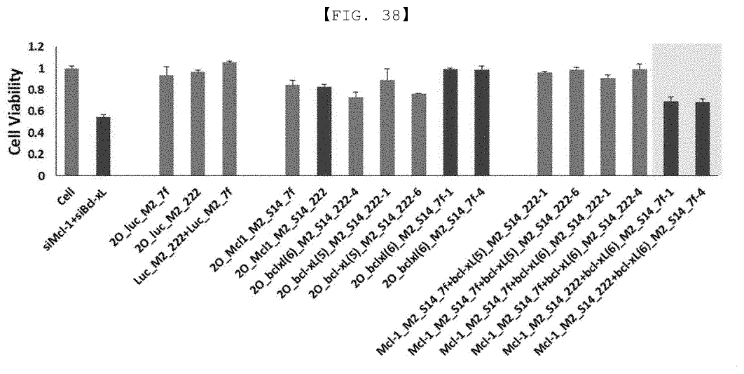

It is expected that only when the two different types of miRNAs exist, the hairpin miRNA triggers prepared above can be opened to repress the expression of the target gene. A schematic diagram illustrating the mechanism in which a hairpin miRNA trigger capable of binding to two types of miRNAs regulates a target gene is depicted in FIG. 36.

The hairpin miRNA triggers prepared above were transfected into MDA-MB-231 and MDA-MB-453 cells which were then measured for cell viability. The results are depicted in FIG. 37. As shown in FIG. 37, greater apoptosis was observed in MDA-MB-231 cells which overexpress both miR-222 let-7f than MDA-MB-453 cells which overexpress let-7f, but not miR-222.

### Example 10. Apoptotic Effect of Hairpin miRNA Trigger on Breast Cancer

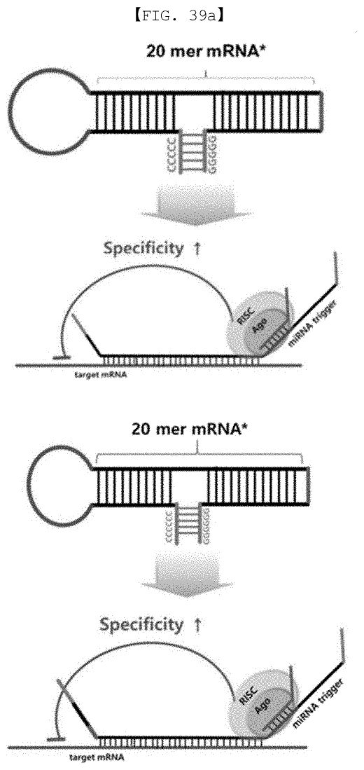

Hairpin miRNA triggers which could bind to miR-222 or let-7f and target mcl-1 or bcl-xL were prepared and transfected into the breast cancer cell line MDA-MB-231 which was then measured for cell viability. The results are depicted in FIG. 38.

The hairpin miRNA triggers used in this Example are listed in Table 26, below. Every nucleotide of the triggers was modified with 2′-O-methyl. In Table 26, the numerals (−1, −4, or −6) used as suffixes in the miRNA trigger names refer to relative positions selected from various numbers of cases allowing the target site for each target gene to have a 14 nt long stem.

TABLE 26

| | | SEQ |
|---|---|---|
| Oligo | Sequence | ID |
| name | (5′->3′) | NO |
| HP_luc_ | mUmCmGmAmAmGmUmAmCmUmCmAmGmCmAmAmC | 121 |
| let7 | mUmAmUmAmCmAmAmUmGmAmAmCmUmAmCmCmU | |
| | mCmAmGmCmUmGmAmGmUmAmCmUmUmCmGmA | |
| HP_luc_ | mUmCmGmAmAmGmUmAmCmUmCmAmGmCmAmCmC | 122 |
| 222 | mCmAmGmUmAmGmCmGmUmGmAmUmGmUmAmGmC | |
| | mUmGmCmUmGmAmGmUmAmCmUmUmCmGmA | |
| HP_Mcl- | mCmCmGmAmAmCmUmAmCmGmUmAmGmCmAmAmC | 123 |
| 1_let7 | mUmAmUmAmCmAmAmUmGmAmAmCmUmAmCmCmU | |
| | mCmAmGmCmUmAmCmGmUmAmGmUmUmCmGmG | |
| HP_Mcl- | mCmCmGmAmAmCmUmAmCmGmUmAmGmCmAmCmC | 124 |
| 1_222 | mCmAmGmUmAmGmCmGmUmGmAmUmGmUmAmGmC | |
| | mUmGmCmUmAmCmGmUmAmGmUmUmCmGmG | |
| HPbcl- | mUmUmCmCmUmGmCmCmCmUmUmmCmCmUmAmCm | 125 |
| xL(5)_ | CmCmAmGmUmAmGmCmGmUmGmAmUmGmUmAmGm | |
| 222-1 | CmUmAmGmGmAmAmGmGmGmCmAmGmGmAmA | |
| HP_bcl- | mUmAmCmCmUmGmCmCmAmGmCmCmUmCmAmCmC | 126 |
| xL(6)_ | mCmAmGmUmAmGmCmGmUmGmAmUmGmUmAmGmC | |
| 222-4 | mUmGmAmGmGmCmUmGmGmCmAmGmGmUmA | |
| HP_bcl- | mUmCmUmCmCmUmUmCmCmUmGmCmCmCmAmCmC | 127 |
| xL(5)_ | mCmAmGmUmAmGmCmGmUmGmAmUmGmUmAmGmC | |
| 222-6 | mUmGmGmGmCmAnGmGmAmAmGmGmAmGmA | |
| HP_bcl- | mCmUmGmCmCmAmGmCmCmUmCmCmUmUmAmAmC | 128 |
| xL(6)_ | mUmAmUmAmCmAmAmUmGmAmAmCmUmAmCmCmU | |
| let7-1 | mCmAmAmAmGmGmAmGmGmCmUmGmGmCmAmG | |
| HP_bcl- | mUmAmCmCmUmGmCmCmAmGmCmCmUmCmAmAmC | 129 |
| xL(6)_ | mUmAmUmAmCmAmAmUmGmAmAmCmUmAmCmCmU | |
| let7-4 | mCmAmGmAmGmGmCmUmGmGmCmAmGmGmUmA | |

As can be seen in FIG. 38, co-transfection of the hairpin miRNA trigger that could bind to miR-222 and target mcl-1 and the hairpin miRNA trigger that could bind to let-7f and target bcl-xL(6) remarkably increased apoptotic effects.

### Example 12. Construction of Dumbbell-Shaped miRNA Trigger

In this Example, a miRNA trigger having increased binding affinity for mRNA of a target gene was constructed. In this regard, the mRNA-recognizing portion of the linear or hairpin miRNA trigger was lengthened from a 14-nt size to a 20-nt size to increase the repressive effect on the expression of the target gene. In order to increase binding affinity for a target gene while retaining the advantage of hairpin miRNA triggers that the expression of the target gene can be repressed only in the presence of a miRNA recognizable by the triggers, the portion capable of complementarily binding to the mRNA of the target gene was lengthened to an about 20-nt size to construct a dumbbell-shaped miRNA trigger (hereinafter referred to as “dumbbell miRNA trigger”). Sequences of dumbbell miRNA triggers are listed in Table 27, below.

TABLE 27

| | | SEQ |
|---|---|---|
| Oligo | Sequence | ID |
| name | (5′->3′) | NO |
| DB_PKRS20_ | CCCCCCGAATGAGAAATACCATC | 130 |
| 141 | TTTACCAGACAGTGTTATATTTC | |
| | TCATTCCCTTCCTT(C12SPACER) | |
| | AAGGAAGGGGGGGG | |
| miRNA-141 | UAACACUGUCUGGUAAAGAUGG | 17 |
| PKR mRNA (2) | CAAUAAUGGGAAGGAAGGGAAU | 73 |
| | GAGAAAUAUUAAAUUCUG | |

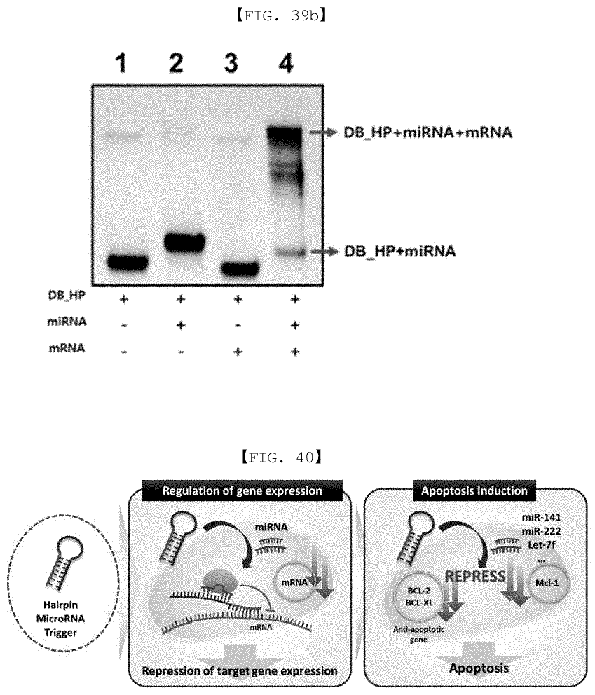

To a buffer (1×PBS buffer, +5 mM MgCl2, 137 mM NaCl, 10 mM PO4, 2.7 mM KCl, 5 mM MgCl₂; pH 7.4) containing 100 nM of the dumbbell miRNA trigger (DP) prepared above, 100 nM miRNA and/or 100 nM target mRNA was added, followed by incubation at 37° C. for 1 hour. Each sample was loaded to a PAGE gel and run. The results are depicted in FIG. 39b.

As shown in FIG. 39b, the dumbbell miRNA trigger according to an embodiment could bind to a miRNA and/or target mRNA only in the presence of the miRNA (lanes 2 and 4 in the gel image of FIG. 39b) and did not bind to a target mRNA in the absence of the miRNA (lane 3 in the gel image of FIG. 39b. From the data, it was understood that the presence of a miRNA of interest is requisite for the binding of the dumbbell miRNA trigger to both the miRNA and the mRNA.

1. A nucleic acid molecule comprising:
an X region capable of binding to a miRNA;
a Y region capable of binding to an mRNA of a target gene; and
a Y* region capable of binding to the Y region,
wherein the Y region and the Y* region complementarily match each other to form a stem-loop structure.

2. The nucleic acid molecule of claim 1,
which is represented by following General Formula 1 or 2:
5′-the Y region-the X region-the Y* region-3′ (General Formula 1)
5′-the Y* region-the X region-the Y region-3′ (General Formula 2).

3. The nucleic acid molecule of claim 1,
wherein the X region comprises a nucleic acid sequence 40% or more complementary to the nucleic acid sequence of the miRNA.

4. The nucleic acid molecule of claim 1,
wherein the Y region comprises a nucleic acid sequence 80% or more complementary to the mRNA of the target gene.

5. The nucleic acid molecule of claim 1,
wherein nucleotide residues of positions 10 and 11 from the 3′ end of the X region is not complementary to the miRNA.

6. The nucleic acid molecule of claim 1, wherein the nucleic acid molecule comprises the X region in the range of one to four.

7. The nucleic acid molecule of claim 6,
wherein the different X regions are capable of binding to the same or different miRNAs.

8. The nucleic acid molecule of claim 1,
wherein the X region consists of 10 to 22 nt nucleotides.

9. The nucleic acid molecule of claim 1,
wherein the Y region consists of 10 to 16 nt nucleotides.

10. The nucleic acid molecule of claim 1,
wherein the Y* region consists of 10 to 16 nt nucleotides.

11. The nucleic acid molecule of claim 1,
wherein a complementary binding between the Y region and the Y* region is dissociated by binding of the miRNA.

12. The nucleic acid molecule of claim 1,
wherein the nucleic acid molecule comprises at least one modified nucleotide or a backbone modification,
wherein the modified nucleotide is a nucleotide residue modified with at least one selected from the group consisting of 2′-O-methyl, 2′-methoxyethoxy, 2′-fluoro, 2′-allyl, 2′-O-[2-(methylamino)-2-oxoethyl], 4′-thio, 4′-CH₂—O-2′-bridge, 4′-(CH₂)₂-O-2′-bridge, 2′-LNA, 2′-amino, and 2′-O—(N-methylcarbamate), and
the backbone modification is at least one selected from the group consisting of phosphonate, phosphorothioate, and phosphotriester.

13. The nucleic acid molecule of claim 1,
wherein the miRNA is coupled with an RISC (RNA-induced silencing complex).

14. The nucleic acid molecule of claim 1,
wherein the miRNA is present in diseased cells, and absent or hypoexpressed in normal cells.

15. The nucleic acid molecule of claim 1,
wherein the miRNA is at least one selected from the group consisting of miR-141, miR-21, miR-200c, miR-222, let-7f, miR-155, miR-24, miR-29a, miR-27a, miR-200a, miR-200b, miR-429, miR-205, miR-30a, miR-34, miR-203, miR-10b, miR-31, miR-9, miR-490, miR-29a, miR-204, miR-221, miR-138, miR-17, miR-19, miR-569, miR-9, miR-22, miR-29b, miR-125b, miR-126, miR-146a, miR-193a, miR-196b, miR-223, miR-492, miR-135b, miR-331, miR-374a, miR-519a, miR-191, miR-210, miR-24, miR-9, miR-27, miR-103, and miR-107

16. The nucleic acid molecule of claim 1,
wherein the target gene is at least one selected from the group consisting of an anti-apoptotic gene, an oncogene, a protooncogene, and an epithelial-mesenchymal transition (EMT) promoting gene.

17. The nucleic acid molecule of claim 1,
wherein the target gene is at least one selected from the group consisting of mcl-1, bcl-xL, bcl-2, Snail1, Twist1, SLUG, Zeb1, TCF4, TCF3, FLT3, STAT3, c-Sis, EGFR, Ras, CYCD, Her2, Myc, Raf; VIM, CDH2, FN1, ACTA2, COL1A1, and SNAI2.

18. A method for repressing expression of a target gene, comprising a step of administering the nucleic acid molecule of claim 1 in an effective amount to a subject.

19. A method for prevention or treatment of a cancer, comprising a step of administering the nucleic acid molecule of claim 1, in a pharmaceutically effective amount to a subject.

20. The method of claim 19, wherein the method induces apoptosis of cancer cells or inhibits metastasis of cancer cells.

21. The method of claim 19, wherein the cancer is at least one solid cancer selected from the group consisting of liver cancer, lung cancer, pancreatic cancer, breast cancer, colon cancer, pancreatic cancer, ovarian cancer, endometrial cancer, cervical cancer, gallbladder cancer, gastric cancer, biliary tract cancer, colorectal cancer, head and neck cancer, thyroid cancer, brain tumor, malignant melanoma, prostate cancer, testicular cancer, and tongue cancer, or
at least one blood cancer selected from the group consisting of leukemias such as chronic lymphocytic leukemia (CLL), acute lymphocytic leukemia (ALL), non-Hodgkin lymphomas (NHL), acute myeloid leukemia (AML), and lymphomas.

22. The method of claim 19, wherein the method further comprises a step of administering an anticancer agent selected from the group consisting of cytarabine, azacitidine, decitabine, paclitaxel, Adriamycin, and tamoxifen.

23. The method of claim 19, wherein the cancer is breast cancer, and the nucleic acid molecule is capable of binding to at least one miRNA selected from the group consisting of miR-222, miR-141, miR-21, miR-200c, miR-222, let-7f, miR-492, miR-135b, miR-331, miR-374a, miR-519a, miR-191, miR-210, and miR-24.

24. The method of claim 23, wherein the nucleic acid molecule is capable of binding to at least one target gene selected from the group consisting of mcl-1, bcl-xL, and bcl-2.

25. The method of claim 19, wherein the cancer is acute myeloid leukemia (AML), and the nucleic acid molecule is capable of binding to at least one miRNA selected from the group consisting of miR-155, miR-21, miR-9, miR-490, miR-29a, miR-204, miR-221, miR-138, miR-17, miR-19, miR-569, miR-9, miR-10b, miR-22, miR-29b, miR-125b, miR-126, miR-146a, miR-193a, miR-196b, and miR-223.

26. The method of claim 25, wherein the nucleic acid molecule is capable of binding to at least one target gene selected from the group consisting of mcl-1, bcl-xL, and bcl-2.

27. The method of claim 19, wherein the cancer is uterine cervical cancer, and the nucleic acid molecule is capable of binding to at least one miRNA selected from the group consisting of miR-141 and miR-200c.

28. The method of claim 27, wherein the nucleic acid molecule is capable of binding to at least one target gene selected from the group consisting of mcl-1, bcl-xL, and bcl-2.
